(19) United States
(12) Patent Application Publication
(10) Pub. No.: US 2016/0346224 A1
(43) Pub. Date: Dec. 1, 2016

(54) COMPOSITIONS AND METHODS FOR REDUCING VISUAL LOSS

(71) Applicant: Edge Therapeutics, Inc., Berkeley Heights, NJ (US)

(72) Inventor: R. Loch Macdonald, Toronto (CA)

(21) Appl. No.: 15/132,922

(22) Filed: Apr. 19, 2016

Related U.S. Application Data
(60) Provisional application No. 62/168,588, filed on May 29, 2015.

(51) Int. Cl. A61K 9/70; A61K 45/06; A61K 9/00; A61K 31/4418

(52) U.S. Cl. CPC A61K 9/7007; A61K 31/4418; A61K 45/06; A61K 9/0048; A61K 9/0051

(57) ABSTRACT

The described invention provides a method for reducing visual loss and for treating one or more of adverse consequence of an eye disease, including abnormal intraocular pressure, retinal vascular disease, retinal ganglion cell death, or a combination thereof in order to reduce visual loss. The method entails providing a flowable particulate composition that contains a particulate formulation comprising a plurality of particles of uniform size distribution, a therapeutic amount of a therapeutic agent selected from a voltage-gated calcium channel antagonist, an endothelin receptor antagonist, or a combination thereof, and optionally an additional therapeutic agent, wherein the particles are of uniform size distribution, and wherein each particle comprises a matrix; and a pharmaceutically acceptable carrier. The pharmaceutical composition is characterized by: dispersal of the therapeutic agent throughout each particle, adsorption of the therapeutic agent onto the particles, or placement of the therapeutic agent in a core surrounded by a coating, sustained release of the therapeutic agent and optionally the additional therapeutic agent from the composition, and a local therapeutic effect that is effective to reduce signs or symptoms of the adverse consequence without entering systemic circulation in an amount to cause unwanted side effects. The method further entails administering a therapeutic amount of the pharmaceutical composition by a means for administration at a site of administration. The administering includes topically, parenterally, or by implantation. Sites of administration include intraocularly, intraorbitally, or into subconjunctival space.

### CROSS-REFERENCE TO RELATED APPLICATION

This application claims the benefit of priority to U.S. Provisional Application No. 62/168,588 filed on May 29, 2015, the entire contents of which are incorporated by reference herein.

### FIELD OF INVENTION

The described invention relates to compositions, systems and methods for reducing visual loss and for treating one or more adverse consequence of an eye disease, including abnormal intraocular pressure, retinal vascular disease, and retinal ganglion cell death, in order to reduce visual loss.

### BACKGROUND OF THE INVENTION

### Glaucoma

Glaucoma, a retinal vascular disease, is the most common optic neuropathy, the second most common cause of blindness, and the most common cause of preventable visual disability worldwide (Chua B. and Goldberg I, Expert Rev Ophthalmol 2010; 5(5); 627-36). It encompasses a spectrum of progressive optic neuropathies characterized by pathological degeneration of nonmyelinated retinal ganglion cells (RGCs) with structural damage at the optic nerve head (Chua B. and Godberg I, Expert Rev Ophthalmol 2010; 5(5); 627-36). The multitude of potential initiating insults triggers a cascade that results in accelerated apoptosis and death from additional mechanisms of the RGCs. It is believed that elevated intraocular pressure (TOP) and vascular insufficiency are primarily responsible for apoptosis and death of RGCs.

### Elevated Intraocular Pressure (TOP)

Elevated IOP often results from alterations in aqueous humor dynamics due to changes in the trabecular meshwork leading to impaired drainage of the aqueous humor. The trabecular meshwork has been shown to exhibit cytoskeletal changes in cells, altered cellularity and changes in extracellular matrix (ECM) with increased IOP (Clark A F, et al., J Glaucoma. 1995; 4:183-8; Alvarado J et al., Ophthalmology. 1984; 91:564-79; Grierson I. What is open angle glaucoma? Eye. 1987; 1:15-28; Lutjen-Drecoll E et al., Exp Eye Res. 1986; 42:443-55; Knepper P A et al., Invest Ophthalmol Vis Sci. 1986; 37:1360-7; Lutjen-Drecoll E et al., In: Ritch R, Shields M B, Krupin T, editors. The glaucomas. St. Louis: Mosby Year; 1996. pp. 89-123). A significant positive correlation has been observed between change in IOP and RGC death in glaucomatous rats and between the level and duration of elevated IOP and RGC axon loss (Morrison J C et al., Exp Eye Res. 1997; 64:85-96; Chauhan B C et al., Invest Ophthalmol Vis Sci. 2002; 43:2969-76; Levkovitch-Verbin H et al., Invest Ophthalmol Vis Sci. 2002; 43:402-10). RGC death in experimental glaucoma has been shown to occur by the process of apoptosis, and IOP elevation can directly induce RGC death by apoptosis (Pease M E et al., Invest Ophthalmol Vis Sci. 2000; 41:764-74; WoldeMussie E et al., Invest Ophthalmol Vis Sci. 2001; 42:2849-55; Garcia-Valenzuela E et al., Exp Eye Res. 1995; 61:33-44). Results of a number of studies suggest that RGC death after exposure to elevated IOP takes place in two phases. The first phase lasts for about three weeks, with loss of approximately 12% RGCs per week. This is believed to be followed by a second slower phase of neuronal loss (WoldeMussie E et al., Invest Ophthalmol Vis Sci. 2001; 42:2849-55). The primary mechanism of neuronal loss in the initial phase is apoptosis while in the second phase neuronal loss is due to toxic effects of the primary degenerating neurons in addition to continuing exposure to elevated IOP (Agar A et al., J Neurosci Res. 2000; 60:495-503; Levkovitch-Verbin H et al., Invest Ophthalmol Vis Sci. 2002; 43:402-10).

### Vascular Insufficiency

In a healthy eye, a constant flow of blood is required in the retina and optic nerve head so as to meet the high metabolic needs in these vital parts of the eye. To maintain a constant rate of blood flow, an efficient autoregulatory mechanism operates in arteries, arterioles and capillaries over a wide range of day-to-day fluctuations in ocular perfusion pressure (OPP) that is dependent on both the systemic blood pressure and IOP (Bill A et al., Eye. 1990; 4:319-25).

### Retrobulbar Blood Flow

The first major branch of the internal carotid artery is the ophthalmic artery (OA) (Harris A et al., Atlas of Ocular Blood Flow: Vascular Anatomy, Pathophysiology, and Metabolism. Butterworth Heinemann Publishers, PA, USA (2003)). The OA progresses to run inferiorly to the ON and enters the orbit through the optic canal. While in the orbit, the OA crosses superior to the ON and continues nasally and anteriorly. The OA terminates after giving off the central retinal artery (CRA) and the posterior ciliary arteries, and branches to the extraocular muscles.

The CRA supplies the inner two-thirds of the retina, the anterior segment of the ONH and portions of the retrolaminar ON (Harris A et al., Atlas of Ocular Blood Flow: Vascular Anatomy, Pathophysiology, and Metabolism. Butterworth Heinemann Publishers, PA, USA (2003)). It penetrates the ON 10-15 mm behind the globe to run adjacent to the central retinal vein in the middle of the ON. The medial and lateral posterior ciliary arteries then branch off the OA. Each posterior ciliary artery further divides to one long posterior ciliary artery (LPCA) and seven to ten short posterior ciliary arteries (SPCAs). The SPCAs supply the peripapillary and posterior choroid, while the LPCA and the anterior ciliary arteries (branches of the muscular arteries) supply the anterior choroid. These retrobulbar vessels provide the majority of the blood to the eye (Harris A et al., Atlas of Ocular Blood Flow: Vascular Anatomy, Pathophysiology, and Metabolism. Butterworth Heinemann Publishers, PA, USA (2003)).

Retrobulbar circulation has been extensively studied with color Doppler imaging (CDI), and there have been numerous studies that have concluded that there is an association between decreased blood flow velocities in the retrobulbar circulation and glaucomatous damage (Martínez A, Sánchez MActa Ophthalmol. Scand. 83(6),716-722 (2005); Galassi F et al., Arch. Ophthalmol. 121(12),1711-1715 (2003); Yamazaki Y, Drance S M. Am. J. Ophthalmol. 124(3),287-295 (1997); Zeitz O et al., Br. J. Ophthalmol. 90(10),1245-1248 (2006); Satilmis M et al., Am. J. Ophthalmol. 135(5),664-669 (2003); Nicolela M T et al., Am. J. Ophthalmol. 121(5),502-510 (1996)). For instance, the retrobulbar vessels in both NTG and primary open angle glaucoma (POAG) patients exhibit increased Pourcelot's index of resistivity (RI) during CDI (Rankin S J et al., Am. J. Ophthalmol. 119(6),685-693 (1995); Netland P A et al., Am. J. Ophthalmol. 115(5),608-613 (1993)). Also, there is a correlation between glaucomatous visual field (VF) progression and decreased blood flow velocities in the SPCAs (Januleviciene I et al., J. Ophthalmol. 2011,164320 (2011); Zeitz O et al., Br. J. Ophthalmol. 90(10),1245-1248 (2006)) and an increased OA RI ((Januleviciene I et al., J. Ophthalmol. 2011,164320 (2011)). This leads to the possibility that reduced blood flow may precede detectable VF damage.

### Retinal Blood Flow

The retina receives its nourishment through intricately arranged blood flow from two sources: the CRA and the uveal system (Harris A et al., Atlas of Ocular Blood Flow: Vascular Anatomy, Pathophysiology, and Metabolism. Butterworth Heinemann Publishers, PA, USA (2003)). The CRA supplies the inner two-thirds of the retina. The uveal system supplies the remainder of the retina by diffusion of molecules from the choroid, through the retinal pigment epithelium, and into the retina, supplying the bipolar cells and photoreceptors. The uveal system will be discussed further when the choroidal blood flow is addressed.

The CRA eventually terminates in four major trunks, each of which supplies a quadrant of the retina (Harris A et al., Atlas of Ocular Blood Flow: Vascular Anatomy, Pathophysiology, and Metabolism. Butterworth Heinemann Publishers, PA, USA (2003)). The retinal arteries and veins course within the RNFL, and eventually the capillaries and fibers of the RNFL run in parallel. Considering that the RGCs are supplied by the retinal circulation and that the RGCs are lost in glaucoma, retinal blood flow is of great importance in understanding the pathophysiology of glaucoma.

Using a Heidelberg Retina Flowmeter (HRF), it has been determined that reductions in retinal blood flow were associated with reductions in visual function (Sato E A et al., Graefes Arch. Clin. Exp. Ophthalmol. 244(7),795-801 (2006)). In patients with asymmetric glaucomatous damage, this technology was used to show that both blood flow and velocity are significantly decreased in eyes with worse damage compared with the fellow eye with less damage (Lam A et al., Curr. Eye Res. 30(3),221-227 (2005)).

### Optic Nerve Head (ONH) Blood Flow

The ONH has a complicated blood supply that originates from several sources. The ONH is separated into four segments: superficial nerve layer, prelaminar region, laminar region and retrolaminar region (Harris A et al., Atlas of Ocular Blood Flow: Vascular Anatomy, Pathophysiology, and Metabolism. Butterworth Heinemann Publishers, PA, USA (2003)). The superficial nerve layer is continuous with the retinal nerve fiber layer (RNFL) and is the only layer visible during the fundus exam. Likewise, the superficial nerve layer is supplied by branches of the retinal arteries. The second region moving posteriorly, the prelaminar region, lies adjacent to the peripapillary choroid. This region is mostly supplied by branches of SPCAs directly and by branches off the circle of Haller and Zinn, an arterial ring formed by SPCA branches. Next is the laminar region, which has the same blood supply as the prelaminar region. The laminar region contains the lamina cribrosa, a connective tissue ring though which the neural fibers pass through. Finally, the retrolaminar region marks the start of axonal myelination. Its blood supply comes from the CRA and the pial system.

Using HRF, it has been determined that blood flow to the peripapillary retina and neuroretinal rim is reduced in glaucoma (Nicolela M T et al., Am. J. Ophthalmol. 122(6),775-783 (1996); Chung H S et al., Br. J. Ophthalmol. 83(4),466-469 (1999)); Jonas J B et al., J. Glaucoma 12(3),260-265 (2003); Hosking S L et al., Br. J. Ophthalmol. 85(11),1298-1302 (2001)). In addition, blood flow at the neuroretinal rim corresponded to regional VF defects in patients with NTG and POAG (Sato EA et al., Graefes Arch. Clin. Exp. Ophthalmol. 244(7),795-801 (2006); Resch H et al., Acta Ophthalmol. 89(7),e544-e549 (2011). Moreover, patients with glaucoma have faulty autoregulation in response to the lowering of IOP (Hafez A S et al., Ophthalmology 110(1), 201-210 (2003)). A study looking at ONH vascular reactivity to normoxic hypercapnia showed that patients with untreated POAG had reduced vascular reactivity compared to healthy controls, thus supporting the concept of vascular dysregulation in glaucoma (Venkataraman S T et al., Invest. Ophthalmol. Vis. Sci. 51(4),2043-2050 (2010). It also has shown that glaucoma patients have reduced total retinal blood flow and increased dye leakage from ONH capillaries, suggesting peripapillary ischemia (Nanba K et al., Ophthalmology 95(9), 1227-1233 (1988); O'Brart D P et al., Am. J. Ophthalmol. 123(5),657-666 (1997)).

### Choroidal Blood Flow

The blood supply to the choroid is divided into anterior and posterior divisions (Harris A et al., Atlas of Ocular Blood Flow: Vascular Anatomy, Pathophysiology, and Metabolism. Butterworth Heinemann Publishers, PA, USA (2003)). The choroid in the posterior half of the globe is supplied by the SPCAs, which also supply much of the ONH. The anterior half of the choroid is supplied by the LPCAs and anterior ciliary arteries. There has not been shown to be any anastomosis between these two circulations, and therefore there is a border zone area where the two circulations meet. The outer choroid contains larger nonfenestrated blood vessels, whereas the inner choroid contains small fenestrated capillaries. The fenestration allows for diffusion of substances into and out of the capillaries and then active transport across the retinal pigment epithelium layer, thus nourishing the outer third of the retina.

Indocyanine green angiography has been used in patients with glaucoma to demonstrate slow choroidal filling and sluggish movement of blood into and out of the choroid (Harris A et al., Ophthal. Imag. Diag. 11,331-337 (1998)). Slowing has also been shown specifically in NTG patients (Duijm H F et al., Am. J. Ophthalmol. 123(5),644-656 (1997)). In addition, Yin et al. found morphological changes in patients with POAG with reduced choroidal thickness due to a reduction in size of the choriocapillaris (Yin Z Q et al., J. Glaucoma 6(1),23-32 (1997)).

Although elevated IOP is thought to play a major role in RGC damage in glaucomatous eyes, therapeutic control of TOP in many patients is not sufficient to improve the visual functions and arrest the progression of the disease process (Rossetti L et al., Arch Ophthalmol. 1993; 111:96-103; Chauhan B C, In: Drance S M, editor. Update to glaucoma, blood flow and drug treatment. Amsterdam: Kugler; 1995. pp. 1-6). This suggests a critical role of other factors, such as vascular insufficiency, in the initiation and progression of glaucomatous changes.

### CSF Flow in the Brain

The cerebrospinal fluid (CSF) is a clear bodily fluid that occupies the ventricular system, subarachnoid space around the brain and spinal cord, and central canal of the spinal cord. CSF is produced by modified ependymal cells of the choroid plexus found throughout the ventricular system. In addition, it is also formed around blood vessels and ventricular walls, presumably from the extracellular space of the brain. CSF flows from the lateral ventricles via interventricular foramina into the third ventricle. CSF then flows into the fourth ventricle through the cerebral aqueduct. CSF flows out in the subarachnoid space via the median aperture and left and right lateral apertures. Finally, the CSF is reabsorbed into the dural venous sinuses through arachnoid granulations and arachnoid villi. Arachnoid granulations consist of collections of villi. The villi are visible herniations of the arachnoid membrane through the dura and into the lumen of the superior sagittal sinus and other venous structures. The granulations appear to function as valves that allow one-way flow of CSF from the subarachnoid spaces into venous blood. All constituents of CSF leave with the fluid, including small molecules, proteins, microorganisms, and red blood cells.

CSF is produced at a rate of approximately 0.3-0.37 ml/minute or 20 ml/hour or 500 ml/day. The volume of the CSF space is about 150 mls and the CSF turns over 3.7 times a day.

The choroid plexus uses capillary filtration and epithelial secretory mechanisms to maintain the chemical stability of the CSF. While the capillaries that traverse the choroid plexus are freely permeable to plasma solutes, a barrier exists at the level of the epithelial cells that make up the choroid plexus, which is responsible for carrier-mediated active transport. CSF and extracellular fluids of the brain are in a steady state and blood plasma and CSF are in osmotic equilibrium under normal physiological conditions.

### Ocular Perfusion Pressure (OPP) as it Relates to Glaucoma

OPP is defined as arterial blood pressure (BP) minus IOP (Araie M, Crowston J, Iwase A et al. Section III: clinical relevance of ocular blood flow (OBF). In: Blood Flow in Glaucoma: The Sixth Consensus Report of the World Glaucoma Association. Weinreb R N, Harris A (Eds). Kugler Publications, Amsterdam, The Netherlands, 60-132 (2009)). Mean ocular perfusion pressure is generally calculated as two-thirds of mean arterial pressure minus IOP (Sehi M et al., Ophthalmol. Vis. Sci. 46(2), 561-567 (2005)). Occasionally, OPP is further divided into systolic perfusion pressure (SBP minus IOP) and diastolic perfusion pressure (DPP; diastolic BP [DBP] minus IOP). Although perfusion pressure changes during the day, to maintain metabolic activity, tissue blood flow should remain stable. Mitra, S., et al., Invest. Ophthalmol. Vis. Sci. 46(2): 561-67 (2005).

Large population-based studies have determined that reduced OPP is strongly associated with increased prevalence of glaucoma (Bonomi L et al., Ophthalmology 107(7), 1287-1293 (2000); Tielsch J M et al., Arch. Ophthalmol. 113(2), 216-221 (1995); Leske M C et al., Arch. Ophthalmol. 120(7),954-959 (2002)). Low DPP has the strongest correlation with the development of glaucoma (Bonomi L et al., Ophthalmology 107(7), 1287-1293 (2000); Leske M C et al., Arch. Ophthalmol. 120(7), 954-959 (2002)). The Baltimore Eye Survey found that those with DPP <30 mmHg had a six-times higher risk of disease development than those with DPP >50 mmHg (Tielsch J M et al., Arch. Ophthalmol. 113(2), 216-221 (1995)). Furthermore, the Barbados Eye Study showed that individuals with the lowest 20% of DPP were 3.3-times more likely to develop glaucoma (Leske M C et al., Arch. Ophthalmol. 113(7),918-924 (1995)). In a subgroup of patients from the Barbados Eye Study followed for 9 years, lower OPPs and lower systolic BPs were again identified as risk factors (Leske M C et al., Ophthalmology 115(1),85-93 (2008); Wozniak K et al., Ophthalmologe 103(12),1027-1031 (2006)). In a different study, low mean ocular perfusion pressure (<42 mmHg), systolic perfusion pressure (<101 mmHg) and DPP (<55 mmHg) were all shown to be risk factors for the development of glaucoma, with relative risks of 3.1, 2.6 and 3.2, respectively (Leske M C, Wu S Y, Nemesure B, Hennis A. Incident open-angle glaucoma and blood pressure. Arch. Ophthalmol. 120(7),954-959 (2002). The Egna-Neumarkt Study reported a 4.5% increase in glaucoma prevalence in patients with DPPs <50 mmHg compared with patients with DPPs ≧66 mmHg (Bonomi et al., Ophthalmology 107(7),1287-1293 (2000)). Despite the fact that these studies are from varying populations, they all found that reduced DPP is an important risk factor for the development of glaucoma.

Evidence suggests that an association exists between vascular insufficiency and glaucoma. A positive association of glaucoma has been observed with peripheral vascular abnormalities that involve dysregulation of cerebral and peripheral vasculature (Gass A et al., Graefes Arch Clin Exp Ophthalmol. 1997; 235:634-8; O'Brien C et al., Ophthalmologica. 1999; 213:150-3). Increased sensitivity to endothelin-l-mediated vasoconstriction is implicated in these vascular abnormalities. The possible role of this vasoconstrictor is also suspected in the pathogenesis of glaucoma as increased levels of endothelin-1 have been detected in the aqueous humor and plasma of glaucoma patients (Cellini M et al., Acta Ophthalmol Scand. 1997; 224:11-3; Noske W et al., Graefes Arch Clin Exp Ophthalmol. 1997; 235:551-2; Tezel G et al., J Glaucoma. 1997; 6:83-9; Hollo Get al., J Glaucoma. 1998; 7:105-10). Further evidence indicating a positive association between glaucoma and vascular insufficiency were provided by magnetic resonance imaging in glaucoma patients revealing pan-cerebral ischemia and increased incidence of cerebral infarcts (Stroman G A et al., Arch Ophthalmol. 1995; 113:168-72; Ong K et al., Ophthalmol. 1995; 102:1632-8. Aging is also considered an important risk factor for glaucoma and a progressive decline in cerebral and ocular perfusion has been observed with increasing age (Nomura H et al., Ophthalmology. 1999; 106:2016-22; Harris A et al., Ophthalmology. 2000; 107:430-4). Autoregulatory mechanisms are not as robust in aging individuals as in youth. Evidence of this can be observed in a study done by Matsuura and Kawai, showing robust choroidal hyperperfusion in response to experimentally induced ocular hypertension in young rats while in older rats a similar increase in choroidal perfusion was not observed (Matsuura K, Kawai Y., Jpn J Physiol. 1998; 48:9-15). Thus, there is evidence to suggest that neuronal damage in glaucoma represents a chronic anterior ischemic optic neuropathy.

### Blood Pressure (BP) and Cerebrospinal Fluid (CSF) Pressure as They Relate to Glaucoma

The Thessaloniki Eye Study assessed the relationship between BP in patients without glaucoma and optic disc morphology, as measured by HRF (Topouzis F et al., Am. J. Ophthalmol. 142(1): 60-67 (2006)). It concluded that being on antihypertensive therapy with DBP <90 mmHg was positively correlated with cup area and cup-to-disc (CD) ratio when compared with both patients with high DBP and patients with untreated, normal diastolic BP (DBP). Also, low OPP was positively associated with cup area and CD ratio. The results did not change after adjusting for cardiovascular disease, diabetes, age, IOP and duration of antihypertensive treatment. These results suggest that BP could be an independent risk factor for glaucomatous damage Topouzis F et al., Am. J. Ophthalmol. 142(1),60-67 (2006)). Also, it brings up the question of whether there is a particular time of day to administer antihypertensive medication for the best treatment outcome (Jonas J B et al., Am. J. Ophthalmol. 142(1),144-145 (2006)). It is unknown whether treating systemic hypertension is better in the morning than at night due to the possibility of worse nocturnal hypotension with treating in the evening.

In addition, the European Glaucoma Prevention Study concluded that the use of systemic diuretics was significantly associated with the development of glaucoma in ocular hypertension (OHT) patients with a hazard ratio of 2.41 (Mills R P, Am. J. Ophthalmol. 144(2),290-291 (2007); Miglior S et al., Am. J. Ophthalmol. 144(2),266-275 (2007)). The combination of antihypertensives with diuretics worsened the prognosis further, with a hazard ratio of 3.07. In contrast to the Thessaloniki Eye Study and the European Glaucoma Prevention Study, systemic hypertension has also been described as a risk factor. This could be due to the association between hypertension and increased IOP. As the Thessaloniki Eye Study adjusted for IOP, it may have more appropriately assessed the relationship between BP and CD ratios (Topouzis F et al., Am. J. Ophthalmol. 142(1),60-67 (2006)). Furthermore, the Thessaloniki Eye Study showed that optic disc changes occurred only when a hypertensive patient was on antihypertensive medication, and thus had normal DBP. Optic disc changes were only found with this combination and were not associated with solely antihypertensive use or BP status, thus implying a connection between these two variables and glaucomatous change.

Recently, more attention has been focused on cerebrospinal fluid (CSF) surrounding the optic nerve. The balance between the anterior force of CSF pressure and the posterior force of IOP in the area of the optic nerve head called the lamina cribrosa is known as the trans-lamina cribrosa pressure difference (Berdahl J P et al., Invest. Ophthalmol. Vis. Sci. 49(12),5412-5418 (2008)). The concern is that variations in this pressure difference can apply damaging force to the optic disc (Nagel E et al., Eur. J. Ophthalmol. 11(4),338-344 (2001)). This is related to blood flow in that CSF pressure is thought to have a positive correlation with BP. With high BPs, CSF pressure rises to prevent dangerously high pressures in the cerebral vasculature. As BP falls, CSF pressure also decreases in order to allow for continued perfusion of the brain and associated structures. Thus, at low BPs, the trans-lamina cribrosa pressure difference is increased due to low CSF pressure. If BP is medically reduced, CSF pressure may also fall so that even with normal IOP, the trans-lamina cribrosa pressure difference will be elevated, such as in high-pressure glaucoma (Topouzis F et al., Am. J. Ophthalmol. 142(1),60-67 (2006)). Furthermore, several studies have concluded that CSF pressure is reduced in some patients with normal tension glaucoma (NTG) and POAG (Berdahl J P et al., Invest. Ophthalmol. Vis. Sci. 49(12),5412-5418 (2008); Berdahl J P et al., Ophthalmology 115(5),763-768 (2008)). Interestingly, some studies have continued to look at the role of CSF in glaucoma and have hypothesized that there is a ‘compartment syndrome’ within the subarachnoid space of the optic nerve (Killer H E et al., Brain 130(Pt 2),514-520 (2007)). It has been proposed that there are variations in CSF pressure on the optic nerve with possible areas of increased pressure. Also, reductions in CSF flow in the region of the optic nerve have resulted in hypotheses that variations in CSF composition, whether it is decreased nutrients or increased toxic metabolites, may be involved in the pathogenesis of optic nerve damage (Id.).

### Ocular Blood Flow as it Relates to Glaucoma

Over the years, many clinical studies have detected ocular blood flow (OBF) deficits in POAG patients. Blood flow parameters in OAG patients have been shown to be reduced in the retrobulbar, retinal, optic nerve head (ONH) and choroidal circulations (Harris A et al., Am. J. Ophthalmol. 118(5),642-649 (1994); Chung H S et al., Br. J. Ophthalmol. 83(4),466-469 (1999); Sato E A et al., Graefes Arch. Clin. Exp. Ophthalmol. 244(7),795-801 (2006); Yin Z Q et al., J. Glaucoma 6(1),23-32 (1997)). These vascular deficits may be one of the first manifestations of glaucoma (Tuulonen A et al., Ophthalmology 94(5),558-563 (1987); Loebl Metal., Arch. Ophthalmol. 95(11),1980-1984 (1977)). Changes in BP and OPP have been associated with OAG. This is also true of other vascular abnormalities such as nocturnal hypotension, optic disc hemorrhage, aging of the vasculature and diabetes (Bonomi L et al., Ophthalmology 107(7),1287-1293 (2000); Tielsch J M et al., Arch. Ophthalmol. 113(2),216-221 (1995); 39.Caprioli J, Coleman A L; Blood Flow in Glaucoma Discussion. Blood pressure, perfusion pressure, and glaucoma. Am. J. Ophthalmol. 149(5),704-712 (2010); Hayreh S S et al., Am. J. Ophthalmol. 117(5),603-624 (1994); Drance S et al., Am. J. Ophthalmol. 131(6),699-708 (2001); Wilensky J T, Surv. Ophthalmol. 41(Suppl. 1),S3-S7 (1996)). Also, vascular dysregulation, which can result in vasospasm, may participate in the pathophysiology of glaucoma (Emre M et al., Br. J. Ophthalmol. 88(5),662-666 (2004); Flammer J, Orgül S., Prog. Retin. Eye Res. 17(2),267-289 (1998)). Vasospasm and systemic hypotension may be distinct risk factors for glaucomatous VF progression (Pache M et al., Eur. J. Ophthalmol. 13(3),260-265 (2003)). It has been proposed that disturbances in OBF in OAG are partly related to systemic vascular dysregulation (J, Orgül S., Prog. Retin. Eye Res. 17(2),267-289 (1998)). Dysfunction of the innermost layer of the blood vessels, the endothelium, is thought to play a role in this vascular dysregulation (Resch H et al., Acta Ophthalmol. 87(1),4-12 (2009)). Vascular tone and blood flow are partially regulated by the endothelium through the release of vasoactive substances such as nitric oxide and endothelin-1. Endothelial dysfunction has been shown in glaucoma by demonstrating an imbalance of vasoactive substances such as nitric oxide and endothelin-1. Flow-mediated vasodilation is decreased in the forearm of NTG patients, indicating that endothelial dysfunction is present in the ocular and systemic vasculature of NTG patients, and is thus not likely only a consequence of the disease process (Resch H et al., Acta Ophthalmol. 87(1),4-12 (2009)).

Despite the fact that evidence from many studies has demonstrated the association between reduced OBF and OAG in various circulations, the current clinical treatment of the disease involves neither documentation nor treatment of the deficits (Wilensky J T. Surv. Ophthalmol. 41(Suppl. 1),S3-S7 (1996)). This is partly due to the need for larger scale clinical studies that will allow the precise relationship between blood flow and glaucomatous damage to be understood.

### Ischemia of Retinal Nerve Ganglion Cells

Glaucoma, being an optic neuropathy, is associated with the loss of retinal ganglion cells (RGCs). With the increased acceptance of the concept of altered blood flow in OAG, one must consider the possibility of ocular tissue ischemia and that ischemia may play a central role in RGC death (Nickells R W. J. Glaucoma 5(5),345-356 (1996); Kyhn M V et al. Exp. Eye Res. 88(6),1100-1106 (2009)). In animal models of glaucoma, RGCs have been shown to die mainly by apoptosis (Nickells R W. J. Glaucoma 5(5),345-356 (1996); Quigley H A et al., Invest. Ophthalmol. Vis. Sci. 36(5),774-786 (1995)). Ischemic injury of RGCs is thought to occur through accumulation of glutamate, leading to glutamate excitotoxicity (Romano C et al., Invest. Ophthalmol. Vis. Sci. 39(2),416-423 (1998)). In vitro models have shown that neuroprotection of the ischemic RGCs can be obtained through blockage of both of the N-methyl-D-aspartate and non-N-methyl-D-aspartate glutamate receptors, or by the delivery of a minimal amount of glucose (Romano C et al., Ophthalmol. Vis. Sci. 39(2),416-423 (1998)). During ischemia, RGC cytoskeleton components have been shown to suffer derangements and could be an important cause of neuronal dysfunction (Balaratnasingam C net al., Invest. Ophthalmol. Vis. Sci. 51(6),3019-3028 (2010)). In addition, these changes are observed before the signs of apoptosis within the RGCs. In the context of ischemia, neuroprotection could be achieved by increasing blood flow, whether it is by better autoregulation or increased OPP. And if indeed RGCs glaucoma are entering apoptosis from ischemia, improving oxygen and nutrient delivery to the eye could offer neuroprotection.

Furthermore, visual function has been correlated to ocular hemodynamics in clinical studies of patients with both diabetes and glaucoma. Contrast sensitivity has been shown to improve with hyperoxia in diabetic patients with substantial initial defect (Harris A et al., Br. J. Ophthalmol. 80(3),209-213 (1996)). Moreover, acute enhancement of ocular perfusion in NTG patients may improve visual function (Pillunat L E et al., In: Glaucoma, Ocular Blood Flow, and Drug Treatment. Drance S M (Ed.). Kugler, Amsterdam, The Netherlands, 67-71 (1995); Bose S et al., Ophthalmology 102(8),1236-1241 (1995); Harris A et al., Am. J. Ophthalmol. 124(3),296-302 (1997)). Similarly, calcium channel antagonists have been shown to benefit visual function acutely and over 6 months in some patients with NTG (Pillunat L E et al., In: Glaucoma, Ocular Blood Flow, and Drug Treatment. Drance S M (Ed.). Kugler, Amsterdam, The Netherlands, 67-71 (1995); Bose S et al., Ophthalmology 102(8),1236-1241 (1995); Harris A et al., Am. J. Ophthalmol. 124(3),296-302 (1997); Kitazawa Y et al., Graefes Arch. Clin. Exp. Ophthalmol. 227(5),408-412 (1989); Sawada A et al., Ophthalmology 103(2),283-288 (1996)). Also, calcium channel antagonists may reduce progression of VF defects in NTG patients (Netland P A et al., Am. J. Ophthalmol. 115(5),608-613 (1993)). While these studies suggest improving ocular perfusion may benefit visual function, the mechanism for the improvement has not been determined.

### Diagnosis of Glaucoma

Clinical signs of glaucoma include, but are not limited to, retinal nerve fiber layer defects, neruoretinal rim thinning with excavation of the optic nerve head (“cupping”) and irreversible acuity and visual field loss. Neural degeneration in glaucoma is not limited to the retina; it also affects neurons in the lateral geniculate nucleus and visual cortex (Chua B. and Goldberg I., Expert Rev Ophthalmol 2010; 5(5):627-36).

### Treatments

In a healthy individual, a delicate balance between vasoconstriction and vasodilation is maintained by endothelin and other vasoconstrictors on the one hand and nitric oxide, prostacyclin and other vasodilators on the other.

### Vasoconstriction and Vasodilation

The term “vasoconstriction” as used herein refers to the narrowing of the blood vessels resulting from contracting of the muscular wall of the vessels. When blood vessels constrict, the flow of blood is restricted or slowed. The term “vasodilation”, which is the opposite of vasoconstriction as used herein, refers to the widening of blood vessels. The terms “vasoconstrictors,” “vasopressors,” or “pressors” as used herein refer to factors causing vasoconstriction. Vasoconstriction usually results in an increase of blood pressure and may be slight or severe. Vasoconstriction may result from disease, medication, or psychological conditions. Medications that cause vasoconstriction include, but are not limited to, catecholamines, antihistamines, decongestants, methylphenidate, cough and cold combinations, pseudoephedrine, and caffeine.

A vasodilator is a drug or chemical that relaxes the smooth muscle in blood vessels causing them to dilate. Dilation of arterial blood vessels (mainly arterioles) leads to a decrease in blood pressure. The relaxation of smooth muscle relies on removing the stimulus for contraction, which depends predominately on intracellular calcium ion concentrations and phosphorylation of myosin light chain (MLC). Thus, vasodilation predominantly works either 1) by lowering intracellular calcium concentration, or 2) by dephosphorylation of MLC, which includes the stimulation of myosin light chain phosphatase and the induction of calcium symporters and antiporters (which pump calcium ions out of the intracellular compartment). The re-uptake of ions into the sarcoplasmic reticulum of smooth muscle via exchangers and expulsion of ions across the plasma membrane also helps to accomplish vasodilation. The specific mechanisms to accomplish these effects vary from vasodilator to vasodilator and may be grouped as endogenous and exogenous. The term “endogenous” as used herein refers to proceeding from within or derived internally; or resulting from conditions within the organism rather than externally caused. The term “exogenous” as used herein refers to originating from outside; derived externally; or externally caused rather than resulting from conditions within the organism.

Vasodilation directly affects the relationship between mean arterial pressure and cardiac output and total peripheral resistance (TPR). Cardiac output may be computed by multiplying the heart rate (in beats/minute) and the stroke volume (the volume of blood ejected during systole). TPR depends on several factors, including, but not limited to, the length of the vessel, the viscosity of blood (determined by hematocrit), and the diameter of the blood vessel. Blood vessel diameter is the most important variable in determining resistance. An increase in either cardiac output or TPR cause a rise in the mean arterial pressure. Vasodilators work to decrease TPR and blood pressure through relaxation of smooth muscle cells in the tunica media layer of large arteries and smaller arterioles.

Vasodilation occurs in superficial blood vessels of warm-blooded animals when their ambient environment is hot; this process diverts the flow of heated blood to the skin of the animal, where heat may be more easily released into the atmosphere. Vasoconstriction is the opposite physiological process. Vasodilation and vasoconstriction are modulated naturally by local paracrine agents produced by endothelial cells (e.g., bradykinin, adenosine), as well as by an organism's autonomic nervous system and adrenal glands, both of which secrete catecholamines, such as norepinephrine and epinephrine, respectively.

Vasodilators are used to treat conditions such as hypertension, where the patient has an abnormally high blood pressure, as well as angina and congestive heart failure, where maintaining a lower blood pressure reduces the patient's risk of developing other cardiac problems.

### TOP Lowering Drugs

Since their introduction in the mid-1990s, prostaglandin analogs have moved to the top of the list for patients with mild to moderately elevated IOP. These drugs are effective at reducing IOP via an enhancement of aqueous humor outflow through the uveo-scleral space. A number of other drugs, including β-adrenergic antagonists and carbonic anhydrase inhibitors, are also available. These agents reduce the production of aqueous humor to lower intraocular pressure and, while effective, require two or three times a day dosing that makes compliance an issue and can significantly compromise therapeutic outcomes. Alpha (α)2 adrenergic agonists also act by decreasing the production of fluid by the ciliary body and enhancing outflow.

### Calcium Channel Antagonists

Nimodipine, a calcium channel antagonist, has been shown in clinical trials to improve ocular blood flow in healthy volunteers and patients with glaucoma (See, e.g., Netland P A et al., Am J Ophthalmol 1995; 119:694-700; Netland P A et al., J Glaucoma 1996; 5:200-6; Yamamoto T et al., J Glaucoma 1998; 7:301-5; Tomita G et al., Int Ophthalmol 1999; 23:3-10). However, calcium channel antagonists are not generally accepted as a therapeutic approach for the prevention of visual field loss in glaucoma patients due to the lack of long-term clinical outcome data from randomized placebo controlled studies.

### Endothelins

Endothelins are vasoconstricting peptides produced primarily in the endothelium and that increase blood pressure and vascular tone. This family of peptides includes endothelin-1 (ET-1), endothelin-2 (ET-2) and endothelin-3 (ET-3). These small peptides (21 amino acids) have an important role in vascular homeostasis. ET-1 is secreted mostly by vascular endothelial cells. The predominant ET-1 isoform is expressed in vasculature and is the most potent vasoconstrictor. ET-1 also has inotropic, chemotactic and mitogenic properties. It stimulates the sympathetic nervous system, and influences salt and water homeostasis through its effects on the renin-angiotensin-aldosterone system (RAAS), vasopressin and atrial natriuretic peptide. Endothelins are among the strongest vasoconstrictors known and have been implicated in vascular diseases of several organ systems, including the heart, general circulation and brain.

There are two key endothelin receptor types, ETA and ETB. ETA and ETB have distinct pharmacological characteristics. The ETA-receptor affinity is much higher for ET-1 than for ET-3. ETA-receptors are located in the vascular smooth muscle cells, but not in endothelial cells. The binding of endothelin to ETA increases vasoconstriction and the retention of sodium, leading to increased blood pressure. ETB receptors primarily are located on the endothelial cells that line the interior of the blood vessels. Endothelin binding to ETB receptors lowers blood pressure by increasing natriuresis and diuresis, and releasing nitric oxide. ET-1 and ET-3 activate the ETB-receptor equally, which in turn leads to vasodilation via production of NO and prostaglandins. Endothelin-1 (ET-1) also has been demonstrated to cause vascular smooth muscle constriction via ETA-receptor stimulation and to induce NO production in endothelial cells via ETB-receptors. Some ETB-receptors are located in vascular smooth muscle, where they may mediate vasoconstriction. A number of endothelin receptors are regulated by various factors. Angiotensin II and phorbol esters down-regulate endothelin receptors whereas ischemia and cyclosporin increase the number of endothelin receptors.

A number of peptide and nonpeptide ET antagonists have been studied. ETA-receptor antagonists may include, without limitation, A-127722 (non-peptide), ABT-627 (non-peptide), BMS 182874 (non-peptide), BQ-123 (peptide), BQ-153 (peptide), BQ-162 (peptide), BQ-485 (peptide), BQ-518 (peptide), BQ-610 (peptide), EMD-122946 (non-peptide), FR 139317 (peptide), IPI-725 (peptide), L-744453 (non-peptide), LU 127043 (non-peptide), LU 135252 (non-peptide), PABSA (non-peptide), PD 147953 (peptide), PD 151242 (peptide), PD 155080 (non-peptide), PD 156707 (non-peptide), RO 611790 (non-peptide), SB-247083 (non-peptide), clazosentan (non-peptide), atrasentan (non-peptide), sitaxsentan sodium (non-peptide), TA-0201 (non-peptide), TBC 11251 (non-peptide), TTA-386 (peptide), WS-7338B (peptide), ZD-1611 (non-peptide), and aspirin (non-peptide). ETA/B-receptor antagonists may include, but are not limited to, A-182086 (non-peptide), CGS 27830 (non-peptide), CP 170687 (non-peptide), J-104132 (non-peptide), L-751281 (non-peptide), L-754142 (non-peptide), LU 224332 (non-peptide), LU 302872 (non-peptide), PD 142893 (peptide), PD 145065 (peptide), PD 160672 (non-peptide), RO-470203 (bosentan, non-peptide), RO 462005 (non-peptide), RO 470203 (non-peptide), SB 209670 (non-peptide), SB 217242 (non-peptide), and TAK-044 (peptide). ETB receptor antagonists may include, but are not limited to, A-192621 (non-peptide), A-308165 (non-peptide), BQ-788 (peptide), BQ-017 (peptide), IRL 1038 (peptide), IRL 2500 (peptide), PD-161721 (non-peptide), RES 701-1 (peptide), and RO 468443 (peptide).

Endothelin antagonists may have a role in the treatment of cardiac, vascular and renal diseases associated with regional or systemic vasoconstriction and cell proliferation, such as essential hypertension, pulmonary hypertension, chronic heart failure and chronic renal failure.

Compliance with Medical Treatment

Despite the availability of therapeutic agents, one of the greatest hurdles in controlling glaucoma is that of compliance. Recent estimates suggest that 60% of glaucoma patients fail to maintain a daily medication regimen (Rossi G C et al., Eur J Ophthalmol 2011; 21:4:410-14). One approach to this problem is to take the task out of the hands of the patient by employing sustained-release or other depot forms of existing drugs. The described invention offers such an approach.

### SUMMARY OF THE INVENTION

According to one aspect, the described invention provides a method for treating at least one adverse consequence of an eye disease comprising abnormal intraocular pressure, retinal vascular disease and retinal ganglion cell death in order to reduce visual loss in a mammal in need thereof, the method comprising: (a) providing a flowable particulate composition comprising (i) a particulate formulation comprising a plurality of particles of uniform size distribution, and a therapeutic amount of a therapeutic agent selected from a voltage-gated calcium channel antagonist, an endothelin receptor antagonist, or a combination thereof, and optionally an additional therapeutic agent, wherein the particles are of uniform size distribution, and wherein each particle comprises a matrix; and (ii) a pharmaceutically acceptable carrier, the pharmaceutical composition being characterized by: dispersal of the therapeutic agent throughout each particle, adsorption of the therapeutic agent onto the particles, or placement of the therapeutic agent in a core surrounded by a coating, sustained release of the therapeutic agent and optionally the additional agent from the composition, and a local therapeutic effect that is effective to reduce signs or symptoms of the adverse consequence without entering systemic circulation in an amount to cause unwanted side effects; and (b) administering a therapeutic amount of the pharmaceutical composition by a means for administration at a site of administration.

According to one embodiment of the method, the adverse consequence of the eye disease comprises abnormal intraocular pressure. According to another embodiment, the adverse consequence of the eye disease comprises retinal ganglion cell death. According to another embodiment, the adverse consequence of the eye disease comprises a retinal vascular disease. According to another embodiment, the retinal vascular disease is glaucoma. According to another embodiment, the voltage-gated calcium channel antagonist is a dihydropyridine. According to another embodiment, the dihydropyridine is nimodipine. According to another embodiment, the additional therapeutic agent is a prostaglandin analog, a Rho kinase inhibitor, or a combination thereof. According to another embodiment, the prostaglandin analog is bimatoprost, latanoprost or travaprost. According to another embodiment, the Rho kinase inhibitor is selected from the group consisting of Y-27632 2HCl (R&D Systems Inc., Minneapolis, Minn.), Triazovivin® (StemRD, Burlingame, Calif.), Slx-2119 (MedChem Express, Namiki Shoji Cop., LTD), WF-536 [(+)-®-4-(1-aminoethyl)-N-(4-pyridyl) benzamide monohydrochloride] (Mitsubishi Pharma Corporation, Osaka, Japan), RK1-1447 (University of South Florida, Tampa, Fla., and Moffitt Cancer Center, Tampa, Fla.; Roberta Pireddu et al., “Pyridylthiazole-based ureas as inhibitors of Rho associated protein kinases (ROCK1 and 2).” (2012) Medchemcomm. 3(6):699-709), Fasudil® (Asahi-KASEI Corp., Osaka, Japan), Fasudil® hydrochloride (R&D Systems Inc., Minneapolis, Minn.), GSK429286A (R&D Systems Inc., Minneapolis, Minn.), Rockout® (EMD Millipore, Philadelphia, Pa.), SR 3677 dihydrochloride (R&D Systems Inc., Minneapolis, Minn.); SB 772077B (R&D Systems Inc., Minneapolis, Minn.), AS 1892802 (R&D Systems Inc., Minneapolis, Minn.), H 1152 dihydrochloride (R&D Systems Inc., Minneapolis, Minn.), GSK 269962 (R&D Systems Inc., Minneapolis, Minn.), HA 1100 hydrochloride (R&D Systems Inc., Minneapolis, Minn.), Glycyl-H-1152 dihydrochloride (R&D Systems Inc., Minneapolis, Minn.), AR-12286 (Aerie Pharmaceuticals), AR-13324 (Rhopressa, Aerie Pharmaceuticals), AMA-0076 (Amakem Therapeutics), and K-115 (Kumatomo University, Japan). According to another embodiment, the administering is topically, parenterally, or by implantation. According to another embodiment, the administering is intraocularly, intraorbitally or into the subconjunctival space. According to another embodiment, the administering intraocularly comprises administering to the vitreous humor, the aqueous humor, or both.

According to another aspect, the described invention provides a kit for treating at least one adverse consequence of an eye disease comprising abnormal intraocular pressure, retinal vascular disease and retinal ganglion cell death in order to reduce visual loss comprising: (a) a flowable particulate composition comprising (i) a particulate formulation comprising a plurality of particles of uniform size distribution, a therapeutic amount of a therapeutic agent selected from a voltage-gated calcium channel antagonist, an endothelin receptor antagonist, or a combination thereof, and optionally an additional therapeutic agent, wherein the microparticles are of uniform size distribution, and wherein each microparticle comprises a matrix, the pharmaceutical composition being characterized by: dispersal of the therapeutic agent throughout each particle, adsorption of the therapeutic agent onto the particles, or placement of the therapeutic agent in a core surrounded by a coating, sustained release of the voltage-gated calcium channel antagonist, the endothelin receptor antagonist, or the combination thereof and optionally an additional therapeutic agent, from the composition, and a local therapeutic effect that is effective to reduce signs or symptoms of the adverse consequence selected from abnormal intraocular pressure, retinal vascular disease and retinal ganglion cell death without entering systemic circulation in an amount to cause unwanted side effects; (b) a means for administering the pharmaceutical composition; and (c) a packaging material. According to one embodiment, the voltage-gated calcium channel antagonist is dihydropyridine. According to another embodiment, the dihydropyridine is nimodipine. According to another embodiment, the pharmaceutical composition further comprises a pharmaceutically acceptable carrier. According to another embodiment, the packaging material is an instruction. According to another embodiment, the means for administering the pharmaceutical composition comprises a syringe, an eye dropper, or a contact lens. According to another embodiment, the contact lens is selected from the group consisting of a soft contact lens, a gas permeable contact lens, and a hybrid contact lens. According to another embodiment, the composition is in a form of a sheet, a string, or a combination thereof. According to another embodiment, the sheet, the string, or a combination thereof is impregnated with the composition.

### BRIEF DESCRIPTION OF THE DRAWINGS

FIG. 1 shows a diagram of the anatomy of the eye. Detail of the retina at the fovea is shown on the right (from Principles of Neural Science, fourth edition; page 508; Eric R. Kandel, James H. Schwartz, Thomas M. Jessell; copyright © 2000 by the McGraw-Hill Companies, Inc., New York, N.Y.).

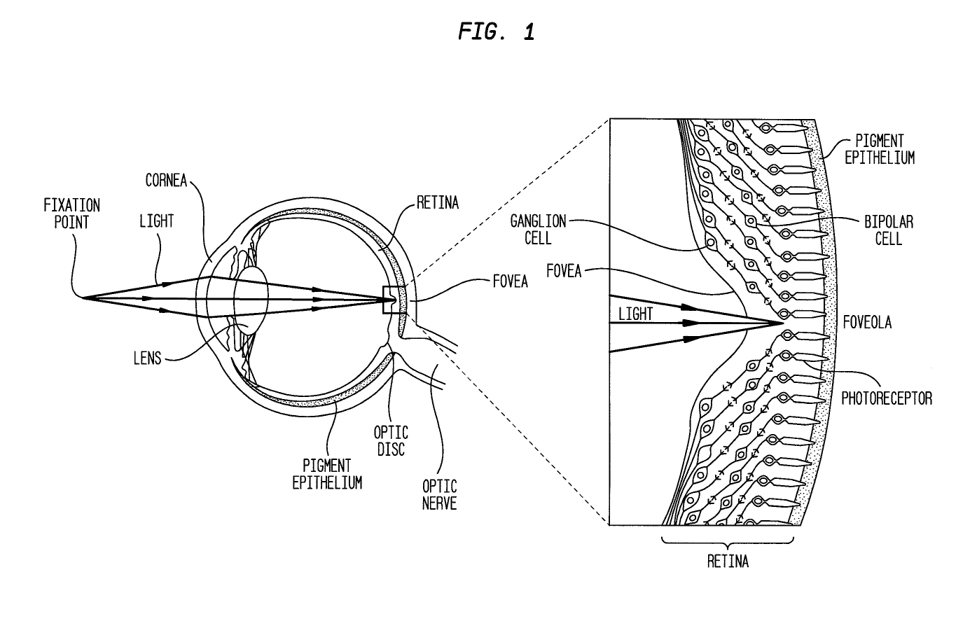

FIG. 2 shows a diagram of arterial blood supply to the eyes and brain (from Correlated Neuroanatomy & Functional Neurology, eighteenth edition; page 325; J. G. Chusid; copyright © 1982 by Lange Medical Publications, Los Altos, Calif.).

FIG. 3 shows a diagram of the anatomy and vasculature of the eye (from Stedman's Medical Dictionary, 27th Edition, page 636 (2000)).

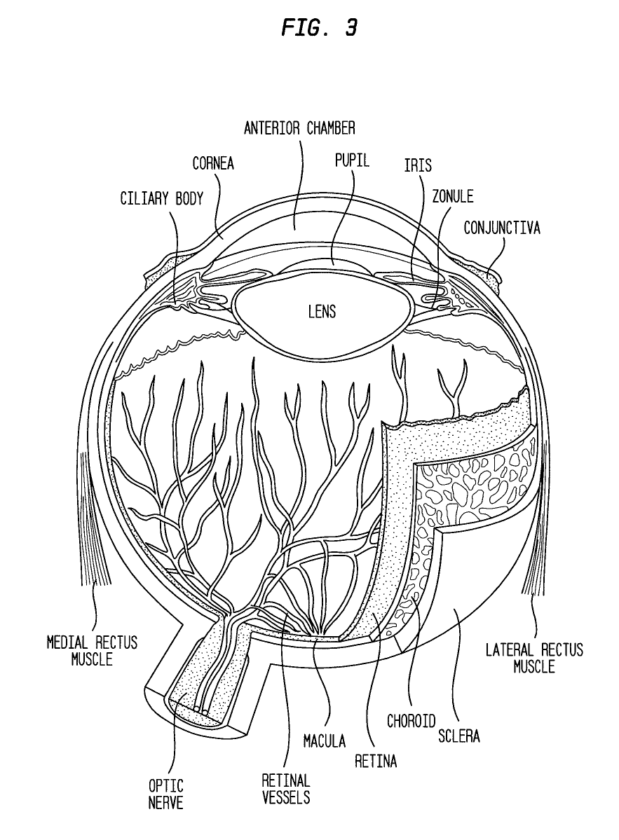

FIG. 4 shows an illustrative view of the CSF flow from the ventricles to the subarachnoid space (page 194, Ross L M, Lamperti E D, Taub E (eds), Schuenke M, Schulte E, Schumacher U. Thieme Atlas of Anatomy. Georg Thieme Verlag: Stuttgart. 2006. pp. 541).

### DETAILED DESCRIPTION OF THE INVENTION

### Glossary

The term “active” as used herein refers to the ingredient, component or constituent of the compositions of the described invention responsible for the intended therapeutic effect.

The term “additional therapeutic agent” as used herein refers to an active ingredient that is intentionally added to the composition of the described invention, which is intended to exert a pharmacological or other beneficial therapeutic effect at the intended dosage.

The term “administering” as used herein includes in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, topical application, or parenterally.

The term “agonist” as used herein refers to a chemical substance capable of activating a receptor to induce a full or partial pharmacological response. Receptors can be activated or inactivated by either endogenous or exogenous agonists and antagonists, resulting in stimulating or inhibiting a biological response. A physiological agonist is a substance that creates the same bodily responses, but does not bind to the same receptor. An endogenous agonist for a particular receptor is a compound naturally produced by the body which binds to and activates that receptor. A superagonist is a compound that is capable of producing a greater maximal response than the endogenous agonist for the target receptor, and thus an efficiency greater than 100%. This does not necessarily mean that it is more potent than the endogenous agonist, but is rather a comparison of the maximum possible response that can be produced inside a cell following receptor binding. Full agonists bind and activate a receptor, displaying full efficacy at that receptor. Partial agonists also bind and activate a given receptor, but have only partial efficacy at the receptor relative to a full agonist. An inverse agonist is an agent which binds to the same receptor binding-site as an agonist for that receptor and reverses constitutive activity of receptors. Inverse agonists exert the opposite pharmacological effect of a receptor agonist. An irreversible agonist is a type of agonist that binds permanently to a receptor in such a manner that the receptor is permanently activated. It is distinct from a mere agonist in that the association of an agonist to a receptor is reversible, whereas the binding of an irreversible agonist to a receptor is believed to be irreversible. This causes the compound to produce a brief burst of agonist activity, followed by desensitization and internalization of the receptor, which with long-term treatment produces an effect more like an antagonist. A selective agonist is specific for one certain type of receptor.

The terms “antagonist” and “inhibitor” are used interchangeably herein to refer to a substance that counteracts the physiological action of another substance.

The term “autoregulation” as used herein refers to the ability to maintain ocular perfusion at constant levels in the face of changing driving force. That is, it maintains ocular perfusion at relatively constant levels over a wide range of systemic blood pressure (BP) and intraocular pressure (TOP).

The term “biocompatible” as used herein refers to causing no clinically relevant tissue irritation, injury, toxic reaction, or immunological reaction to living tissue.

The term “biodegradable” as used herein refers to material that will break down actively or passively over time by simple chemical processes, by action of body enzymes or by other similar biological activity mechanisms.

The term “carrier” as used herein describes a material that does not cause significant irritation to an organism and does not abrogate the biological activity and properties of the active compound of the composition of the described invention. Carriers must be of sufficiently high purity and of sufficiently low toxicity to render them suitable for administration to the mammal being treated. The carrier can be inert, or it can possess pharmaceutical benefits, cosmetic benefits or both. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components.

The phrase “in close proximity” as used herein refers to within less than 10 mm, less than 9.9 mm, less than 9.8 mm, less than 9.7 mm, less than 9.6 mm, less than 9.5 mm, less than 9.4 mm, less than 9.3 mm, less than 9.2 mm, less than 9.1 mm, less than 9.0 mm, less than 8.9 mm, less than 8.8 mm, less than 8.7 mm, less than 8.6 mm, less than 8.5 mm, less than 8.4 mm, less than 8.3 mm, less than 8.2 mm, less than 8.1 mm, less than 8.0 mm, less than 7.9 mm, less than 7.8 mm, less than 7.7 mm, less than 7.6 mm, less than 7.5 mm, less than 7.4 mm, less than 7.3 mm, less than 7.2 mm, less than 7.1 mm, less than 7.0 mm, less than 6.9 mm, less than 6.8 mm, less than 6.7 mm, less than 6.6 mm, less than 6.5 mm, less than 6.4 mm, less than 6.3 mm, less than 6.2 mm, less than 6.1 mm, less than 6.0 mm, less than 5.9 mm, less than 5.8 mm, less than 5.7 mm, less than 5.6 mm, less than 5.5 mm, less than 5.4 mm, less than 5.3 mm, less than 5.2 mm, less than 5.1 mm, less than 5.0 mm, less than 4.9 mm, less than 4.8 mm, less than 4.7 mm, less than 4.6 mm, less than 4.5 mm, less than 4.4 mm, less than 4.3 mm, less than 4.2 mm, less than 4.1 mm, less than 4.0 mm, less than 3.9 mm, less than 3.8 mm, less than 3.7 mm, less than 3.6 mm, less than 3.5 mm, less than 3.4 mm, less than 3.3 mm, less than 3.2 mm, less than 3.1 mm, less than 3.0 mm, less than 2.9 mm, less than 2.8 mm, less than 2.7 mm, less than 2.6 mm, less than 2.5 mm, less than 2.4 mm, less than 2.3 mm, less than 2.2 mm, less than 2.1 mm, less than 2.0 mm, less than 1.9 mm, less than 1.8 mm, less than 1.7 mm, less than 1.6 mm, less than 1.5 mm, less than 1.4 mm, less than 1.3 mm, less than 1.2 mm, less than 1.1 mm, less than 1.0 mm, less than 0.9 mm, less than 0.8 mm, less than 0.7 mm, less than 0.6 mm, less than 0.5 mm, less than 0.4 mm, less than 0.3 mm, less than 0.2 mm, less than 0.1 mm, less than 0.09 mm, less than 0.08 mm, less than 0.07 mm, less than 0.06 mm, less than 0.05 mm, less than 0.04 mm, less than 0.03 mm, less than 0.02 mm, less than 0.01 mm, less than 0.009 mm, less than 0.008 mm, less than 0.007 mm, less than 0.006 mm, less than 0.005 mm, less than 0.004 mm, less than 0.003 mm, less than 0.002 mm, less than 0.001 mm of a site of retinal vascular disease or into a blood vessel in close proximity to a site of retinal vascular disease.

The term “condition”, as used herein, refers to a variety of health states and is meant to include disorders or diseases caused by any underlying mechanism or disorder, injury, and the promotion of healthy tissues and organs.

The term “contact” and all its grammatical forms as used herein refers to a state or condition of touching or of immediate or local proximity.

The term “controlled release” is intended to refer to any drug-containing formulation in which the manner and profile of drug release from the formulation are regulated. This refers to immediate as well as non-immediate release formulations, with non-immediate release formulations including, but not limited to, sustained release and delayed release formulations.

The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.”

The term “diffuse pharmacologic effect”, as used herein, refers to a pharmacologic effect that spreads, disperses or scatters widely over a space or surface.

The term “disease” or “disorder”, as used herein, refers to an impairment of health or a condition of abnormal functioning.

The term “disposed”, as used herein, refers to being placed, arranged or distributed in a particular fashion.

The term “dropper” as used herein refers to a pipet consisting of a small tube with a vacuum bulb at one end for drawing liquid in and releasing it a drop at a time.

The term “drug” as used herein refers to a therapeutic agent or any substance, other than food, used in the prevention, diagnosis, alleviation, treatment, or cure of disease.

The term “effective amount” refers to the amount necessary or sufficient to realize a desired biologic effect.

The term “emulsion” as used herein refers to a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil.

The term “flowable”, as used herein, refers to that which is capable of movement in or as if in a stream by continuous change of relative position.

The term “hydrogel” as used herein refers to a substance resulting in a solid, semisolid, pseudoplastic, or plastic structure containing a necessary aqueous component to produce a gelatinous or jelly-like mass.

The term “hypertension” as used herein refers to high systemic blood pressure; transitory or sustained elevation of systemic blood pressure to a level likely to induce cardiovascular damage or other adverse consequences.

The term “hypotension” as used herein refers to subnormal systemic arterial blood pressure; reduced pressure or tension of any kind.

The term “implanting” as used herein refers to grafting, embedding or inserting a substance, composition, or device into a pre-determined location within a tissue.

The term “impregnate”, as used herein in its various grammatical forms refers to causing to be infused or permeated throughout; to fill interstices with a substance.

The term “infarction” as used herein refers to a sudden insufficiency of arterial or venous blood supply due to emboli, thrombi, mechanical factors, or pressure that produces a macroscopic area of necrosis.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “injury,” as used herein, refers to damage or harm to a structure or function of the body caused by an outside agent or force, which may be physical or chemical.

The term “ischemia” as used herein refers to a lack of blood supply and oxygen that occurs when reduced perfusion pressure distal to an abnormal narrowing (stenosis) of a blood vessel is not compensated by autoregulatory dilation of the resistance vessels. Because the zone least supplied generally is the farthest out, ischemia generally appears in areas farthest away from the blood supply.

The term “isolated molecule” as used herein refers to a molecule that is substantially pure and is free of other substances with which it is ordinarily found in nature or in vivo systems to an extent practical and appropriate for its intended use.

The terms “in the body”, “void volume”, “resection pocket”, “excavation”, “injection site”, “deposition site” or “implant site” or “site of delivery” as used herein are meant to include all tissues of the body without limit, and may refer to spaces formed therein from injections, surgical incisions, tumor or tissue removal, tissue injuries, abscess formation, or any other similar cavity, space, or pocket formed thus by action of clinical assessment, treatment or physiologic response to disease or pathology as non-limiting examples thereof.

The phrase “localized administration”, as used herein, refers to administration of a therapeutic agent in a particular location in the body that may result in a localized pharmacologic effect or a diffuse pharmacologic effect.

The phrase “localized pharmacologic effect”, as used herein, refers to a pharmacologic effect limited to a certain location, i.e. in close proximity to a certain location, place, area or site.

The term “long-term” release, as used herein, means that an implant is constructed and arranged to deliver therapeutic levels of the active ingredient for at least 7 days, and potentially up to about 30 to about 60 days.

The term “modulate” as used herein means to regulate, alter, adapt, or adjust to a certain measure or proportion.

The term “optionally”, as used herein, means something that may be or is chosen.

The term “parenteral” as used herein refers to introduction into the body by way of an injection (i.e., administration by injection) outside the gastrointestinal tract, including, for example, subcutaneously (i.e., an injection beneath the skin), intramuscularly (i.e., an injection into a muscle); intravenously (i.e., an injection into a vein); intraventricularly (i.e., an injection into a cerebral ventricle); intracisternally (i.e., an injection into a cerebral cistern); intrathecally (i.e., an injection into the space around the spinal cord or under the arachnoid membrane of the brain), or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

The terms “particles” or “microparticles”, as used herein, refer to extremely small constituents, e.g., nanoparticles or microparticles) that may contain in whole or in part at least one therapeutic agent as described herein. The particles may contain the therapeutic agent(s) in a core surrounded by a coating. Therapeutic agent(s) also may be dispersed throughout the particles. Therapeutic agent(s) also may be adsorbed into the particles. The particles may be of any order release kinetics, including zero order release, first order release, second order release, delayed release, sustained release, immediate release, etc., and any combination thereof. The particle may include, in addition to therapeutic agent(s), any of those materials routinely used in the art of pharmacy and medicine, including, but not limited to, erodible, nonerodible, biodegradable, or nonbiodegradable material or combinations thereof. The particles may be microcapsules that contain the voltage-gated calcium channel antagonist in a solution or in a semi-solid state. The particles may be of virtually any shape.

The term “pharmaceutically acceptable carrier” as used herein refers to one or more compatible solid or liquid filler, diluents or encapsulating substances which are suitable for administration to a human or other vertebrate animal. The term “carrier” as used herein refers to an organic or inorganic ingredient, natural or synthetic, with which the active ingredient is combined to facilitate the application. The components of the pharmaceutical compositions also are capable of being commingled in a manner such that there is no interaction which would substantially impair the desired pharmaceutical efficiency.

The term “pharmaceutical composition” is used herein to refer to a composition that is employed to prevent, reduce in intensity, cure or otherwise treat a target condition or disease.

The term “pharmacologic effect”, as used herein, refers to a result or consequence of exposure to an active agent.

The phrase “predominantly localized pharmacologic effect”, as used herein, refers to a pharmacologic effect of a drug limited to a certain location by at least 1 to 3 orders of magnitude achieved with a localized administration as compared to a systemic administration.

The term “prognosis” as used herein refers to an expected future cause and outcome of a disease or disorder, based on medical knowledge.

The term “reduce” or “reducing” as used herein refers to a diminution, a decrease, an attenuation, limitation or abatement of the degree, intensity, extent, size, amount, density, number or occurrence of the disorder in individuals at risk of developing the disorder.

The term “subacute inflammation” as used herein refers to a tissue reaction typically seen subsequent to the early inflammatory process characterized by a mixture of neutrophils, lymphocytes, and occasionally macrophages and/or plasma cells.

The terms “subject” or “individual” or “patient” are used interchangeably to refer to a member of an animal species of mammalian origin, including humans.

The phrase “substantially pure” as used herein refers to a condition of a therapeutic agent such that it has been substantially separated from the substances with which it may be associated in living systems or during synthesis. According to some embodiments, a substantially pure therapeutic agent is at least 70% pure, at least 75% pure, at least 80% pure, at least 85% pure, at least 90% pure, at least 95% pure, at least 96% pure, at least 97% pure, at least 98% pure, or at least 99% pure.

The term “sustained release” (also referred to as “extended release”) is used herein in its conventional sense to refer to a drug formulation that provides for gradual release of a drug over an extended period of time, and that preferably, although not necessarily, results in substantially constant local or blood levels of a drug over an extended time period. Alternatively, delayed absorption of a parenterally administered drug form is accomplished by dissolving or suspending the drug in an oil vehicle. Nonlimiting examples of sustained release biodegradable polymers include polyesters, polyester polyethylene glycol copolymers, polyamino-derived biopolymers, polyanhydrides, polyorthoesters, polyphosphazenes, sucrose acetate dibutyrate (SAIB), photopolymerizable biopolymers, protein polymers, collagen, polysaccharides, chitosans, and alginates.

The term “syndrome,” as used herein, refers to a pattern of symptoms indicative of some disease or condition.

The phrase “systemic administration”, as used herein, refers to administration of a therapeutic agent with a pharmacologic effect on the entire body. Systemic administration includes enteral administration (e.g. oral) through the gastrointestinal tract and parenteral administration (e.g. intravenous, intramuscular, etc.) outside the gastrointestinal tract.

The term “therapeutic amount” or an “amount effective” of one or more of the active agents is an amount that is sufficient to provide the intended benefit of treatment. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen may be planned which does not cause substantial toxicity and yet is effective to treat the particular subject. A therapeutically effective amount of the active agents that can be employed ranges from generally 0.1 mg/kg body weight and about 50 mg/kg body weight. Therapeutically effective amount for any particular application may vary depending on such factors as the disease or condition being treated, the particular voltage-gated calcium channel antagonist being administered, the size of the subject, or the severity of the disease or condition. One of ordinary skill in the art may determine empirically the effective amount of a particular inhibitor and/or other therapeutic agent without necessitating undue experimentation. It is preferred generally that a maximum dose be used, that is, the highest safe dose according to some medical judgment. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a surgeon using standard methods. “Dose” and “dosage” are used interchangeably herein.

The term “therapeutic agent” as used herein refers to a drug, molecule, nucleic acid, protein, composition or other substance that provides a therapeutic effect. The terms “therapeutic agent” and “active agent” are used interchangeably. The active agent may be a calcium channel inhibitor, a calcium channel antagonist, a calcium channel blocker and, optionally, an additional therapeutic agent. According to some embodiments, the therapeutic agent(s) may be provided in particles.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The term “topical” refers to administration of a composition at, or immediately beneath, the point of application. The phrase “topically applying” describes application onto one or more surfaces(s) including epithelial surfaces. Topical administration, in contrast to transdermal administration, generally provides a local rather than a systemic effect.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “vasoconstriction” as used herein refers to the narrowing of the blood vessels resulting from contracting of the muscular wall of the vessels. When blood vessels constrict, the flow of blood is restricted or slowed.

The term “vasodilation” which is the opposite of vasoconstriction as used herein refers to the widening of blood vessels. The terms “vasoconstrictors,” “vasopressors,” or “pressors” as used herein refer to factors causing vasoconstriction.

### I. Compositions

According to one aspect, the described invention provides a pharmaceutical composition comprising (i) a microparticulate formulation of a voltage-gated calcium channel antagonist; an endothelin receptor antagonist, or a combination thereof; and optionally (ii) a pharmaceutically acceptable carrier.

According to some embodiments, the pharmaceutical composition is effective to prevent or reduce the incidence or severity of a retinal vascular disease.

According to some embodiments, the pharmaceutical composition, when administered in a therapeutic amount at a site of delivery in the mammal, is effective in preventing or reducing the incidence or severity of a retinal vascular disease.

According to some embodiments, the site of delivery is in proximity to a blood vessel 10 mm, less than 10 mm, less than 9.9 mm, less than 9.8 mm, less than 9.7 mm, less than 9.6 mm, less than 9.5 mm, less than 9.4 mm, less than 9.3 mm, less than 9.2 mm, less than 9.1 mm, less than 9.0 mm, less than 8.9 mm, less than 8.8 mm, less than 8.7 mm, less than 8.6 mm, less than 8.5 mm, less than 8.4 mm, less than 8.3 mm, less than 8.2 mm, less than 8.1 mm, less than 8.0 mm, less than 7.9 mm, less than 7.8 mm, less than 7.7 mm, less than 7.6 mm, less than 7.5 mm, less than 7.4 mm, less than 7.3 mm, less than 7.2 mm, less than 7.1 mm, less than 7.0 mm, less than 6.9 mm, less than 6.8 mm, less than 6.7 mm, less than 6.6 mm, less than 6.5 mm, less than 6.4 mm, less than 6.3 mm, less than 6.2 mm, less than 6.1 mm, less than 6.0 mm, less than 5.9 mm, less than 5.8 mm, less than 5.7 mm, less than 5.6 mm, less than 5.5 mm, less than 5.4 mm, less than 5.3 mm, less than 5.2 mm, less than 5.1 mm, less than 5.0 mm, less than 4.9 mm, less than 4.8 mm, less than 4.7 mm, less than 4.6 mm, less than 4.5 mm, less than 4.4 mm, less than 4.3 mm, less than 4.2 mm, less than 4.1 mm, less than 4.0 mm, less than 3.9 mm, less than 3.8 mm, less than 3.7 mm, less than 3.6 mm, less than 3.5 mm, less than 3.4 mm, less than 3.3 mm, less than 3.2 mm, less than 3.1 mm, less than 3.0 mm, less than 2.9 mm, less than 2.8 mm, less than 2.7 mm, less than 2.6 mm, less than 2.5 mm, less than 2.4 mm, less than 2.3 mm, less than 2.2 mm, less than 2.1 mm, less than 2.0 mm, less than 1.9 mm, less than 1.8 mm, less than 1.7 mm, less than 1.6 mm, less than 1.5 mm, less than 1.4 mm, less than 1.3 mm, less than 1.2 mm, less than 1.1 mm, less than 1.0 mm, less than 0.9 mm, less than 0.8 mm, less than 0.7 mm, less than 0.6 mm, less than 0.5 mm, less than 0.4 mm, less than 0.3 mm, less than 0.2 mm, less than 0.1 mm, less than 0.09 mm, less than 0.08 mm, less than 0.07 mm, less than 0.06 mm, less than 0.05 mm, less than 0.04 mm, less than 0.03 mm, less than 0.02 mm, less than 0.01 mm, less than 0.009 mm, less than 0.008 mm, less than 0.007 mm, less than 0.006 mm, less than 0.005 mm, less than 0.004 mm, less than 0.003 mm, less than 0.002 mm, less than 0.001 mm of a site of retinal vascular disease.

According to some embodiments, one half of the therapeutic agent is released from the pharmaceutical composition at the site of delivery within at least 1 day to at least 365 days in vivo. According to one embodiment, one-half of the therapeutic agent is released from the pharmaceutical composition at the site of delivery within 1 day. According to another embodiment, one-half of the therapeutic agent is released from the pharmaceutical composition at the site of delivery within 2 days. According to another embodiment, one-half of the therapeutic agent is released from the pharmaceutical composition at the site of delivery within 3 days. According to another embodiment, one-half of the therapeutic agent is released from the pharmaceutical composition at the site of delivery within 4 days. According to another embodiment, one-half of the therapeutic agent is released from the pharmaceutical composition at the site of delivery within 5 days. According to another embodiment, one-half of the therapeutic agent is released from the pharmaceutical composition at the site of delivery within 6 days. According to another embodiment, one-half of the therapeutic agent is released from the pharmaceutical composition at the site of delivery within 7 days. According to another embodiment, one-half of the therapeutic agent is released from the pharmaceutical composition at the site of delivery within 8 days. According to another embodiment, one-half of the therapeutic agent is released from the pharmaceutical composition at the site of delivery within 9 days. According to another embodiment, one-half of the therapeutic agent is released from the pharmaceutical composition at the site of delivery within 10 days. According to another embodiment, one-half of the therapeutic agent is released from the pharmaceutical composition at the site of delivery within 15 days. According to another embodiment, one-half of the therapeutic agent is released from the pharmaceutical composition at the site of delivery within 20 days. According to another embodiment, one-half of the therapeutic agent is released from the pharmaceutical composition at the site of delivery within 30 days. According to another embodiment, one-half of the therapeutic agent is released from the pharmaceutical composition at the site of delivery within 40 days. According to another embodiment, one-half of the therapeutic agent is released from the pharmaceutical composition at the site of delivery within a half-life of 50 days. According to another embodiment, one-half of the therapeutic agent is released from the pharmaceutical composition at the site of delivery within 60 days. According to another embodiment, one-half of the therapeutic agent is released from the pharmaceutical composition at the site of delivery within 70 days. According to another embodiment, one-half of the therapeutic agent is released from the pharmaceutical composition at the site of delivery within 80 days. According to another embodiment, one-half of the therapeutic agent is released from the pharmaceutical composition at the site of delivery within 90 days. According to another embodiment, one-half of the therapeutic agent is released from the pharmaceutical composition at the site of delivery within 100 days. According to another embodiment, one-half of the therapeutic agent is released from the pharmaceutical composition at the site of delivery within 110 days. According to another embodiment, one-half of the therapeutic agent is released from the pharmaceutical composition at the site of delivery within 120 days. According to another embodiment, one-half of the therapeutic agent is released from the pharmaceutical composition at the site of delivery within 140 days. According to another embodiment, one-half of the therapeutic agent is released from the pharmaceutical composition at the site of delivery within 150 days. According to another embodiment, one-half of the therapeutic agent is released from the pharmaceutical composition at the site of delivery within 160 days. According to another embodiment, one-half of the therapeutic agent is released from the pharmaceutical composition at the site of delivery within 170 days. According to another embodiment, one-half of the therapeutic agent is released from the pharmaceutical composition at the site of delivery within 180 days. According to another embodiment, one-half of the therapeutic agent is released from the pharmaceutical composition at the site of delivery within 190 days. According to another embodiment, one-half of the therapeutic agent is released from the pharmaceutical composition at the site of delivery within 200 days. According to another embodiment, one-half of the therapeutic agent is released from the pharmaceutical composition at the site of delivery within 210 days. According to another embodiment, one-half of the therapeutic agent is released from the pharmaceutical composition at the site of delivery within 220 days. According to another embodiment, one-half of the therapeutic agent is released from the pharmaceutical composition at the site of delivery within 230 days. According to another embodiment, one-half of the therapeutic agent is released from the pharmaceutical composition at the site of delivery within 240 days. According to another embodiment, one-half of the therapeutic agent is released from the pharmaceutical composition at the site of delivery within 250 days. According to another embodiment, one-half of the therapeutic agent is released from the pharmaceutical composition at the site of delivery within 260 days. According to another embodiment, one-half of the therapeutic agent is released from the pharmaceutical composition at the site of delivery within 270 days. According to another embodiment, one-half of the therapeutic agent is released from the pharmaceutical composition at the site of delivery within 280 days. According to another embodiment, one-half of the therapeutic agent is released from the pharmaceutical composition at the site of delivery within 290 days. According to another embodiment, one-half of the therapeutic agent is released from the pharmaceutical composition at the site of delivery within 300 days. According to another embodiment, one-half of the therapeutic agent is released from the pharmaceutical composition at the site of delivery within 310 days. According to another embodiment, one-half of the therapeutic agent is released from the pharmaceutical composition at the site of delivery within 310 days. According to another embodiment, one-half of the therapeutic agent is released from the pharmaceutical composition at the site of delivery within 320 days. According to another embodiment, one-half of the therapeutic agent is released from the pharmaceutical composition at the site of delivery within 330 days. According to another embodiment, one-half of the therapeutic agent is released from the pharmaceutical composition at the site of delivery within 340 days. According to another embodiment, one-half of the therapeutic agent is released from the pharmaceutical composition at the site of delivery within 350 days. According to another embodiment, one-half of the therapeutic agent is released from the pharmaceutical composition at the site of delivery within 360 days. According to another embodiment, one-half of the therapeutic agent is released from the pharmaceutical composition at the site of delivery within at least 360 days.

According to another embodiment, the release of the therapeutic agent at the site of delivery can produce a predominantly localized pharmacologic effect over a desired amount of time. According to one embodiment, the release of the therapeutic agent at the site of delivery produces a predominantly localized pharmacologic effect for at least 1 day. According to one embodiment, the release of the therapeutic agent at the site of delivery produces a predominantly localized pharmacologic effect for at least 2 days. According to one embodiment, the release of the therapeutic agent at the site of delivery produces a predominantly localized pharmacologic effect for at least 3 days. According to one embodiment, the release of the therapeutic agent at the site of delivery produces a predominantly localized pharmacologic effect for at least 4 days. According to one embodiment, the release of the therapeutic agent at the site of delivery produces a predominantly localized pharmacologic effect for at least 5 days. According to one embodiment, the release of the therapeutic agent at the site of delivery produces a predominantly localized pharmacologic effect for at least 6 days. According to one embodiment, the release of the therapeutic agent at the site of delivery produces a predominantly localized pharmacologic effect for at least 7 days. According to one embodiment, the release of the therapeutic agent at the site of delivery produces a predominantly localized pharmacologic effect for at least 8 days. According to one embodiment, the release of the therapeutic agent at the site of delivery produces a predominantly localized pharmacologic effect for at least 9 days. According to one embodiment, the release of the therapeutic agent at the site of delivery produces a predominantly localized pharmacologic effect for at least 10 days. According to one embodiment, the release of the therapeutic agent at the site of delivery produces a predominantly localized pharmacologic effect for at least 15 days. According to one embodiment, the release of the therapeutic agent at the site of delivery produces a predominantly localized pharmacologic effect for at least 20 days. According to one embodiment, the release of the therapeutic agent at the site of delivery produces a predominantly localized pharmacologic effect for at least 5 days. According to one embodiment, the release of the therapeutic agent at the site of delivery produces a predominantly localized pharmacologic effect for at least 30 days. According to one embodiment, the release of the therapeutic agent at the site of delivery produces a predominantly localized pharmacologic effect for at least 35 days. According to one embodiment, the release of the therapeutic agent at the site of delivery produces a predominantly localized pharmacologic effect for at least 40 days. According to one embodiment, the release of the therapeutic agent at the site of delivery produces a predominantly localized pharmacologic effect for at least 45 days. According to one embodiment, the release of the therapeutic agent at the site of delivery produces a predominantly localized pharmacologic effect for at least 50 days. According to one embodiment, the release of the therapeutic agent at the site of delivery produces a predominantly localized pharmacologic effect for at least 55 days. According to one embodiment, the release of the therapeutic agent at the site of delivery produces a predominantly localized pharmacologic effect for at least 60 days. According to one embodiment, the release of the therapeutic agent at the site of delivery produces a predominantly localized pharmacologic effect for at least 70 days. According to one embodiment, the release of the therapeutic agent at the site of delivery produces a predominantly localized pharmacologic effect for at least 80 days. According to one embodiment, the release of the therapeutic agent at the site of delivery produces a predominantly localized pharmacologic effect for at least 90 days. According to one embodiment, the release of the therapeutic agent at the site of delivery produces a predominantly localized pharmacologic effect for at least 100 days. According to one embodiment, the release of the therapeutic agent at the site of delivery produces a predominantly localized pharmacologic effect for at least 110 days. According to one embodiment, the release of the therapeutic agent at the site of delivery produces a predominantly localized pharmacologic effect for at least 120 days. According to one embodiment, the release of the therapeutic agent at the site of delivery produces a predominantly localized pharmacologic effect for at least 130 days. According to one embodiment, the release of the therapeutic agent at the site of delivery produces a predominantly localized pharmacologic effect for at least 140 days. According to one embodiment, the release of the therapeutic agent at the site of delivery produces a predominantly localized pharmacologic effect for at least 150 days. According to one embodiment, the release of the therapeutic agent at the site of delivery produces a predominantly localized pharmacologic effect for at least 160 days. According to one embodiment, the release of the therapeutic agent at the site of delivery produces a predominantly localized pharmacologic effect for at least 170 days. According to one embodiment, the release of the therapeutic agent at the site of delivery produces a predominantly localized pharmacologic effect for at least 180 days. According to one embodiment, the release of the therapeutic agent at the site of delivery produces a predominantly localized pharmacologic effect for at least 190 days. According to one embodiment, the release of the therapeutic agent at the site of delivery produces a predominantly localized pharmacologic effect for at least 200 days. According to one embodiment, the release of the therapeutic agent at the site of delivery produces a predominantly localized pharmacologic effect for at least 210 days. According to one embodiment, the release of the therapeutic agent at the site of delivery produces a predominantly localized pharmacologic effect for at least 220 days. According to one embodiment, the release of the therapeutic agent at the site of delivery produces a predominantly localized pharmacologic effect for at least 230 days. According to one embodiment, the release of the therapeutic agent at the site of delivery produces a predominantly localized pharmacologic effect for at least 240 days. According to one embodiment, the release of the therapeutic agent at the site of delivery produces a predominantly localized pharmacologic effect for at least 250 days. According to one embodiment, the release of the therapeutic agent at the site of delivery produces a predominantly localized pharmacologic effect for at least 260 days. According to one embodiment, the release of the therapeutic agent at the site of delivery produces a predominantly localized pharmacologic effect for at least 270 days. According to one embodiment, the release of the therapeutic agent at the site of delivery produces a predominantly localized pharmacologic effect for at least 280 days. According to one embodiment, the release of the therapeutic agent at the site of delivery produces a predominantly localized pharmacologic effect for at least 290 days. According to one embodiment, the release of the therapeutic agent at the site of delivery produces a predominantly localized pharmacologic effect for at least 300 days. According to one embodiment, the release of the therapeutic agent at the site of delivery produces a predominantly localized pharmacologic effect for at least 310 days. According to one embodiment, the release of the therapeutic agent at the site of delivery produces a predominantly localized pharmacologic effect for at least 320 days. According to one embodiment, the release of the therapeutic agent at the site of delivery produces a predominantly localized pharmacologic effect for at least 330 days. According to one embodiment, the release of the therapeutic agent at the site of delivery produces a predominantly localized pharmacologic effect for at least 340 days. According to one embodiment, the release of the therapeutic agent at the site of delivery produces a predominantly localized pharmacologic effect for at least 350 days. According to one embodiment, the release of the therapeutic agent at the site of delivery produces a predominantly localized pharmacologic effect for at least 360 days.

According to another embodiment, the release of the therapeutic agent at the site of delivery produces a diffuse pharmacologic effect throughout the eye over a desired amount of time. According to another embodiment, the release of the therapeutic agent can produce a diffuse pharmacologic effect throughout the eye for at least 1 day. According to another embodiment, the release of the therapeutic agent can produce a diffuse pharmacologic effect throughout the eye for at least 2 days. According to another embodiment, the release of the therapeutic agent can produce a diffuse pharmacologic effect throughout the eye for at least 3 days. According to another embodiment, the release of the therapeutic agent can produce a diffuse pharmacologic effect throughout the eye for at least 4 days. According to another embodiment, the release of the therapeutic agent can produce a diffuse pharmacologic effect throughout the eye for at least 5 days. According to another embodiment, the release of the therapeutic agent can produce a diffuse pharmacologic effect throughout the eye for at least 6 days. According to another embodiment, the release of the therapeutic agent can produce a diffuse pharmacologic effect throughout the eye for at least 7 days. According to another embodiment, the release of the therapeutic agent can produce a diffuse pharmacologic effect throughout the eye for at least 8 days. According to another embodiment, the release of the therapeutic agent can produce a diffuse pharmacologic effect throughout the eye for at least 9 days. According to another embodiment, the release of the therapeutic agent can produce a diffuse pharmacologic effect throughout the eye for at least 10 days. According to another embodiment, the release of the therapeutic agent can produce a diffuse pharmacologic effect throughout the eye for at least 15 days. According to another embodiment, the release of the therapeutic agent can produce a diffuse pharmacologic effect throughout the eye for at least 15 days. According to another embodiment, the release of the therapeutic agent can produce a diffuse pharmacologic effect throughout the eye for at least 20 days. According to another embodiment, the release of the therapeutic agent can produce a diffuse pharmacologic effect throughout the eye for at least 25 days. According to another embodiment, the release of the therapeutic agent can produce a diffuse pharmacologic effect throughout the eye for at least 30 days. According to another embodiment, the release of the therapeutic agent can produce a diffuse pharmacologic effect throughout the eye for at least 35 days. According to another embodiment, the release of the therapeutic agent can produce a diffuse pharmacologic effect throughout the eye for at least 40 days. According to another embodiment, the release of the therapeutic agent can produce a diffuse pharmacologic effect throughout the eye for at least 45 days. According to another embodiment, the release of the therapeutic agent can produce a diffuse pharmacologic effect throughout the eye for at least 50 days. According to another embodiment, the release of the therapeutic agent can produce a diffuse pharmacologic effect throughout the eye for at least 55 days. According to another embodiment, the release of the therapeutic agent can produce a diffuse pharmacologic effect throughout the eye for at least 60 days. According to another embodiment, the release of the therapeutic agent can produce a diffuse pharmacologic effect throughout the eye for at least 70 days. According to another embodiment, the release of the therapeutic agent can produce a diffuse pharmacologic effect throughout the eye for at least 80 days. According to another embodiment, the release of the therapeutic agent can produce a diffuse pharmacologic effect throughout the eye for at least 90 days. According to another embodiment, the release of the therapeutic agent can produce a diffuse pharmacologic effect throughout the eye for at least 100 days. According to another embodiment, the release of the therapeutic agent can produce a diffuse pharmacologic effect throughout the eye for at least 110 days. According to another embodiment, the release of the therapeutic agent can produce a diffuse pharmacologic effect throughout the eye for at least 120 days. According to another embodiment, the release of the therapeutic agent can produce a diffuse pharmacologic effect throughout the eye for at least 130 days. According to another embodiment, the release of the therapeutic agent can produce a diffuse pharmacologic effect throughout the eye for at least 140 days. According to another embodiment, the release of the therapeutic agent can produce a diffuse pharmacologic effect throughout the eye for at least 150 days. According to another embodiment, the release of the therapeutic agent can produce a diffuse pharmacologic effect throughout the eye for at least 160 days. According to another embodiment, the release of the therapeutic agent can produce a diffuse pharmacologic effect throughout the eye for at least 170 days. According to another embodiment, the release of the therapeutic agent can produce a diffuse pharmacologic effect throughout the eye for at least 180 days. According to another embodiment, the release of the therapeutic agent can produce a diffuse pharmacologic effect throughout the eye for at least 190 days. According to another embodiment, the release of the therapeutic agent can produce a diffuse pharmacologic effect throughout the eye for at least 200 days. According to another embodiment, the release of the therapeutic agent can produce a diffuse pharmacologic effect throughout the eye for at least 210 days. According to another embodiment, the release of the therapeutic agent can produce a diffuse pharmacologic effect throughout the eye for at least 220 days. According to another embodiment, the release of the therapeutic agent can produce a diffuse pharmacologic effect throughout the eye for at least 230 days. According to another embodiment, the release of the therapeutic agent can produce a diffuse pharmacologic effect throughout the eye for at least 240 days. According to another embodiment, the release of the therapeutic agent can produce a diffuse pharmacologic effect throughout the eye for at least 250 days. According to another embodiment, the release of the therapeutic agent can produce a diffuse pharmacologic effect throughout the eye for at least 260 days. According to another embodiment, the release of the therapeutic agent can produce a diffuse pharmacologic effect throughout the eye for at least 270 days. According to another embodiment, the release of the therapeutic agent can produce a diffuse pharmacologic effect throughout the eye for at least 280 days. According to another embodiment, the release of the therapeutic agent can produce a diffuse pharmacologic effect throughout the eye for at least 290 days. According to another embodiment, the release of the therapeutic agent can produce a diffuse pharmacologic effect throughout the eye for at least 300 days. According to another embodiment, the release of the therapeutic agent can produce a diffuse pharmacologic effect throughout the eye for at least 310 days. According to another embodiment, the release of the therapeutic agent can produce a diffuse pharmacologic effect throughout the eye for at least 320 days. According to another embodiment, the release of the therapeutic agent can produce a diffuse pharmacologic effect throughout the eye for at least 330 days. According to another embodiment, the release of the therapeutic agent can produce a diffuse pharmacologic effect throughout the eye for at least 340 days. According to another embodiment, the release of the therapeutic agent can produce a diffuse pharmacologic effect throughout the eye for at least 350 days. According to another embodiment, the release of the therapeutic agent can produce a diffuse pharmacologic effect throughout the eye for at least 360 days.

According to one embodiment, the pharmaceutical composition is effective to increase ocular blood flow as compared to a control. According to another embodiment, the pharmaceutical composition is effective to increase ocular blood flow, optic nerve blood flow, optic nerve head blood flow, retrobulbar blood flow, retinal blood flow, choroidal blood flow, ocular perfusion or a combination thereof.

According to one embodiment, the diffuse pharmacologic effect is a reduction of a vasospasm such that internal diameter of a blood vessel that is at least 10 mm, at least 9.9 mm, at least 9.8 mm, at least 9.7 mm, at least 9.6 mm, at least 9.5 mm, at least 9.4 mm, at least 9.3 mm, at least 9.2 mm, at least 9.1 mm, at least 9.0 mm, at least 8.9 mm, at least 8.8 mm, at least 8.7 mm, at least 8.6 mm, at least 8.5 mm, at least 8.4 mm, at least 8.3 mm, at least 8.2 mm, at least 8.1 mm, at least 8.0 mm, at least 7.9 mm, at least 7.8 mm, at least 7.7 mm, at least 7.6 mm, at least 7.5 mm, at least 7.4 mm, at least 7.3 mm, at least 7.2 mm, at least 7.1 mm, at least 7.0 mm, at least 6.9 mm, at least 6.8 mm, at least 6.7 mm, at least 6.6 mm, at least 6.5 mm, at least 6.4 mm, at least 6.3 mm, at least 6.2 mm, at least 6.1 mm, at least 6.0 mm, at least 5.9 mm, at least 5.8 mm, at least 5.7 mm, at least 5.6 mm, at least 5.5 mm, at least 5.4 mm, at least 5.3 mm, at least 5.2 mm, at least 5.1 mm, at least 5.0 mm from the site of delivery is increased as compared to a control.

### Antagonists and Inhibitors of Calcium Channels

Calcium channel antagonists are a class of drugs and natural substances having effects on many excitable cells of the body, such as the muscle of the heart, smooth muscles of the vessels or neuron cells. The main action of calcium channel antagonists is to decrease blood pressure.

Most calcium channel antagonists decrease the force of contraction of the myocardium. This is known as the “negative inotropic effect” of calcium channel antagonists. Most calcium channel antagonists are not the preferred choice of treatment in individuals with cardiomyopathy due to their negative inotropic effects.

Many calcium channel antagonists slow the conduction of electrical activity within the heart by blocking the calcium channel during the plateau phase of the action potential of the heart. This “negative dromotropic effect” causes a lowering of the heart rate and may cause heart blocks (which is known as the “negative chronotropic effect” of calcium channel antagonists). The negative chronotropic effects of calcium channel antagonists make them a commonly used class of agents for control of the heart rate in individuals with atrial fibrillation or flutter.

Calcium channel antagonists act upon voltage-gated calcium channels (VGCCs) in muscle cells of the heart and blood vessels. By blocking the calcium channel they prevent large increases of the calcium levels in the cells when stimulated, which subsequently leads to less muscle contraction. In the heart, a decrease in calcium available for each beat results in a decrease in cardiac contractility. In blood vessels, a decrease in calcium results in less contraction of the vascular smooth muscle and therefore an increase in blood vessel diameter. The resultant vasodilation decreases total peripheral resistance (TPR), while a decrease in cardiac contractility decreases cardiac output. Since blood pressure is in part determined by cardiac output and peripheral resistance, blood pressure drops.

Calcium channel antagonists do not decrease the responsiveness of the heart to input from the sympathetic nervous system. Since blood pressure regulation is carried out by the sympathetic nervous system (via the baroreceptor reflex), calcium channel antagonists allow blood pressure to be maintained more effectively than do β-antagonists. However, because calcium channel antagonists result in a decrease in blood pressure, the baroreceptor reflex often initiates a reflexive increase in sympathetic activity leading to increased heart rate and contractility. The decrease in blood pressure also likely reflects a direct effect of antagonism of VDCC in vascular smooth muscle, leading to vasodilation. A β-blocker may be combined with a calcium channel antagonist to minimize these effects.

The antagonists for L, N, and P/Q-types of calcium channels are utilized in distinguishing channel subtypes. For the R-type calcium channel subtype, ω-agatoxin IIIA shows blocking activity, even though its selectivity is rather low. This peptide binds to all of the high voltage-activated channels including L, N, and P/Q subtypes (J. Biol. Chem., 275, 21309 (2000)). A putative R-type (or class alE) selective antagonist, SNX-482, a toxin from the tarantula Hysterocrates gigas, is a 41 amino acid residue peptide with 3 disulfide linkages (1-4, 2-5 and 3-6 arrangement) (Biochemistry, 37, 15353 (1998), Peptides 1998, 748 (1999)). This peptide blocks the class E calcium channel (IC50=15 nM to 30 nM) and R-type calcium current in the neurohypophysial nerve endings at 40 nM concentration. R-type (class E) calcium channel blocking activity is highly selective; no effect is observed on K+ and Na+ currents, and L, P/Q and T-type calcium currents. N-type calcium current is blocked only weakly 30-50% at 300 nM to 500 nM. Regionally, different sensitivity of R-type current to SNX-482 is observed; no significant effect on R-type current occurs in preparations of the neuronal cell body, retinal ganglion cells and hippocampal pyramidal cells. Using SNX-482, three alpha E-calcium subunits with distinct pharmacological properties are recognized in cerebellar R-type calcium channels (J. Neurosci., 20, 171 (2000)). Similarly, it has been shown that secretion of oxytocin, but not vasopressin, is regulated by R-type calcium current in neurohypophysial terminals (J. Neurosci., 19, 9235 (1999)).

Dihydropyridine calcium channel antagonists often are used to reduce systemic vascular resistance and arterial pressure, but are not used to treat angina (with the exception of amlodipine, which carries an indication to treat chronic stable angina as well as vasospastic angina) since the vasodilation and hypotension can lead to reflex tachycardia. This calcium channel antagonist class is easily identified by the suffix “-dipine.”

Phenylalkylamine calcium channel antagonists are relatively selective for myocardium. They reduce myocardial oxygen demand and reverse coronary vasospasm. They have minimal vasodilatory effects compared with dihydropyridines. Their action is intracellular.

Benzothiazepine calcium channel antagonists are an intermediate class between phenylalkylamine and dihydropyridines in their selectivity for vascular calcium channels. Benzothiazepines are able to reduce arterial pressure without producing the same degree of reflex cardiac stimulation caused by dihydropyridines due to their cardiac depressant and vasodilator actions.

L-type VDCC inhibitors are calcium entry blocking drugs whose main pharmacological effect is to prevent or slow entry of calcium into cells via L-type voltage-gated calcium channels. Examples of L-type calcium channel inhibitors include but are not limited to: dihydropyridine L-type antagonists, such as nisoldipine, nicardipine, nilvadipine, and nifedipine, AHF (such as 4aR,9aS)-(+)-4a-Amino-1,2,3,4,4a,9a-hexahydro-4a14-fluorene, HCl), isradipine (such as 4-(4-Benzofurazanyl)-1,-4-dihydro-2,6-dimethyl-3,5-pyridinedicarboxylic acid methyl 1-methhylethyl ester), calciseptine (such as isolated from (Dendroaspis polylepis ploylepis), H-Arg-Ile-Cys-Tyr-Ile-His-Lys-Ala-Ser-Leu-Pro-Arg-Ala-Thr-Lys-Thr-LysVal-Gly-Asn-Thr-Cys-Tyr-Lys-Met-Phe-Ile-Arg-Thr-Gln- Arg-Glu-Tyr-Ile-Ser-Glu-Arg-Gly-Cys-Gly-Cys-Pro-Thr-Ala-Met-Trp-Pro-Tyr-Gl-n-Thr-Glu-Cys-Cys-Lys-Gly-Asp-Cys-Asn-Lys-Oh, Calcicludine (such as isolated from Dendroaspis angusticeps (eastern green mamba)),(H-Trp-Gln-Pro-Pro-Trp-Tyr-Cys-Lys-Glu-Pro-Val-Arg-Ile-Gly-Ser-Cys-Lys-Lys-Gln-Phe-Ser-Ser-Phe-Tyr-Phe-Lys-Trp-Thr-Ala-Lys-Lys-Cys-Leu- Pro-Phe-Leu-Phe-Ser-Gly-Cys-Gly-Gly-Asn-Ala-Asn-Arg-Phe-Gln-Thr-Ile-Gly-Glu-Cys-Arg-Lys-Lys-Cys-Leu-Gly-Lys-OH, Cilnidipine (such as also FRP-8653, a dihydropyridine-type inhibitor), Dilantizem (such as (2S,3S)-(+)-cis-3-Acetoxy-5-(2-dimethylaminoethyl)-2,3-dihydro-2-(4-methoxyphenyl)-1,5-benzothiazepin-4(5H)-one hydrochloride), diltiazem (such as benzothiazepin-4(5H)-one, 3-(acetyloxy)-5-[2-(dimethylamino)ethyl1]-2,3-dihydro-2-(4-methoxyphenyl)-,(+)-cis-, monohydrochloride), Felodipine (such as 4-(2,3-Dichlorophenyl)-1,4-dihydro-2,6-dimethyl-3,5-pyridinecarboxylic acid ethyl methyl ester), FS-2 (such as an isolate from Dendroaspis polylepis polylepis venom), FTX-3.3 (such as an isolate from Agelenopsis aperta), Neomycin sulfate (such as C₂₃H₄₆N₆O₁₃·3H₂SO₄), Nicardipine (such as 1,4-Dihydro-2,6-dimethyl-4-(3-nitrophenypmethyl-2-[methyl(phenylmethypamino]-3,5-pyridinedicarboxylic acid ethyl ester hydrochloride, also YC-93, Nifedipine (such as 1,4-Dihydro-2,6-dimethyl-4-(2-nitrophenyl)-3,5-pyridinedicarboxylic acid dimethyl ester), Nimodipine (such as 4-Dihydro-2,6-dimethyl-4-(3-nitrophenyl)-3,5-pyridinedicarboxylic acid 2-methoxyethyl 1-methylethyl ester) or (Isopropyl 2-methoxyethyl 1,4-dihydro-2,6-dimethyl-4-(m-nitrophenyl)-3,5-pyridinedicarboxylate), Nitrendipine (such as 1,4-Dihydro-2,6-dimethyl-4-(3-nitrophenyl)-3,5-pyridinedicarboxylic acid ethyl methyl ester), S-Petasin (such as (3 S,4aR,5R,6R)-[2,3,4,4a,5,6,7,8-Octahydro-3-(2-propenyl)-4a,5-dimethyl-2-o-xo-6-naphthyl]Z-3′-methylthio-1′-propenoate), Phloretin (such as 2′,4′,6′-Trihydroxy-3-(4-hydroxyphenyl)propiophenone, also 3-(4-Hydroxyphenyl)-1-(2,4,6-trihydroxyphenyl)-1-propanone, also b-(4-Hydroxyphenyl)-2,4,6-trihydroxypropiophenone), Protopine (such as C₂₀H₁₉NO₅Cl), SKF-96365 (such as 1-[b-[3-(4-Methoxyphenyl)propoxy]-4-methoxyphenethyl]-1H-imidazole, HCl), Tetrandine (such as 6,6′,7,12-Tetramethoxy-2,2′-dimethylberbaman), (.+−.)-Methoxyverapamil or (+)-Verapamil (such as 54N-(3,4-Dimethoxyphenylethyl)methylamino]-2-(3,4-dimethoxyphenyl)-2-iso-propylvaleronitrile hydrochloride), and (R)-(+)-Bay K8644 (such as R-(+)-1,4-Dihydro-2,6-dimethyl-5-nitro-442-(trifluoromethyl)phenyl]-3-py-ridinecarboxylic acid methyl ester). The foregoing examples may be specific to L-type voltage-gated calcium channels or may inhibit a broader range of voltage-gated calcium channels, e.g. N, P/Q, R, and T-type.

According to some embodiments, the voltage-gated channel antagonist is selected from the group consisting of L-type voltage-gated calcium channel antagonist, N-type voltage-gated calcium channel antagonist, P/Q-type voltage-gated calcium channel antagonist, or a combination thereof.

Non-limiting examples of therapeutic agents that can be formulated into the composition include, but are not limited to, L-type voltage-gated calcium channel antagonists, N-type voltage-gated calcium channel antagonists, P/Q-type voltage-gated calcium channel antagonists, or a combination thereof.

According to some embodiments, the voltage-gated calcium channel antagonist is a dihydropyridine calcium channel antagonist. According to one embodiment, the dihydropyridine calcium channel antagonist is nimodipine. According to one embodiment, the nimodipine has a half-life of 7-10 days when formulated as described herein, and appropriate lipid solubility.

According to some embodiments, the therapeutic agent is an isolated molecule. The term “isolated molecule” as used herein refers to a molecule that is substantially pure and is free of other substances with which it is ordinarily found in nature or in vivo systems to an extent practical and appropriate for its intended use.

According to some embodiments, the therapeutic agent is admixed with a pharmaceutically-acceptable carrier in a pharmaceutical preparation. According to some such embodiments, the therapeutic agent comprises only a small percentage by weight of the preparation. According to some embodiments, the therapeutic agent is substantially pure.

According to some embodiments, ETA-receptor antagonists may include, but are not limited to, A-127722 (non-peptide), ABT-627 (non-peptide), BMS 182874 (non-peptide), BQ-123 (peptide), BQ-153 (peptide), BQ-162 (peptide), BQ-485 (peptide), BQ-518 (peptide), BQ-610 (peptide), EMD-122946 (non-peptide), FR 139317 (peptide), IPI-725 (peptide), L-744453 (non-peptide), LU 127043 (non-peptide), LU 135252 (non-peptide), PABSA (non-peptide), PD 147953 (peptide), PD 151242 (peptide), PD 155080 (non-peptide), PD 156707 (non-peptide), RO 611790 (non-peptide), SB-247083 (non-peptide), clazosentan (non-peptide), atrasentan (non-peptide), sitaxsentan sodium (non-peptide), TA-0201 (non-peptide), TBC 11251 (non-peptide), TTA-386 (peptide), WS-7338B (peptide), ZD-1611 (non-peptide), and aspirin (non-peptide). ETA/B-receptor antagonists may include, but are not limited to, A-182086 (non-peptide), CGS 27830 (non-peptide), CP 170687 (non-peptide), J-104132 (non-peptide), L-751281 (non-peptide), L-754142 (non-peptide), LU 224332 (non-peptide), LU 302872 (non-peptide), PD 142893 (peptide), PD 145065 (peptide), PD 160672 (non-peptide), RO-470203 (bosentan, non-peptide), RO 462005 (non-peptide), RO 470203 (non-peptide), SB 209670 (non-peptide), SB 217242 (non-peptide), and TAK-044 (peptide). ETB-ireceptor antagonists may include, but are not limited to, A-192621 (non-peptide), A-308165 (non-peptide), BQ-788 (peptide), BQ-017 (peptide), IRL 1038 (peptide), IRL 2500 (peptide), PD-161721 (non-peptide), RES 701-1 (peptide), and RO 468443 (peptide).

According to one embodiment, the flowable particulate pharmaceutical composition comprising (i) a microparticulate formulation of a voltage-gated calcium channel antagonist; an endothelin receptor antagonist, or a combination thereof; and optionally (ii) a pharmaceutically acceptable carrier further comprises a therapeutic amount of one or more additional therapeutic agent(s). According to some embodiments, the additional therapeutic agent is a prostaglandin analog. According to some embodiments, the additional therapeutic agent is one or more Rho kinase inhibitor.

The term “derivative” or “analog” as used herein refers to a compound (e.g., a small molecule compound or a peptide) that may be produced from another compound of similar structure in one or more steps. A “derivative” or “derivatives” of a compound retains at least a degree of the desired function of the compound. Accordingly, an alternate term for “derivative” or “analog” may be “functional derivative.” Derivatives can include chemical modifications, such as akylation, acylation, carbamylation, iodination or any modification that derivatizes the compound. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives. The imidazole nitrogen of histidine can be derivatized to form N-im-benzylhistidine. Also included as derivatives or analogs are those peptides that contain one or more naturally occurring amino acid derivative of the twenty standard amino acids, for example, 4-hydroxyproline, 5-hydroxylysine, 3-methylhistidine, homoserine, ornithine or carboxyglutamiate, and can include amino acids that are not linked by peptide bonds. Such peptide derivatives can be incorporated during synthesis of a peptide, or a peptide can be modified by well-known chemical modification methods (see, e.g., Glazer et al., Chemical Modification of Proteins, Selected Methods and Analytical Procedures, Elsevier Biomedical Press, New York (1975)).

### Prostaglandin Analogs

Prostaglandins are a family of a group of lipid compounds that are derived enzymatically in the body from essential fatty acids. Every prostaglandin contains 20 carbon atoms, including a 5-carbon ring. Prostaglandins have a wide variety of effects, including, but not limited to, muscular constriction mediating inflammation, calcium movement, hormone regulation and cell growth control. Prostaglandins act on a variety of cells, including vascular smooth muscle cells (causing constriction or dilation), platelets (causing aggregation or disaggregation), and spinal neurons (causing pain).

The basic chemical structure of naturally occurring prostaglandins, as shown below, reveals that prostaglandins generally consist of a cyclopentane ring and two side chains:

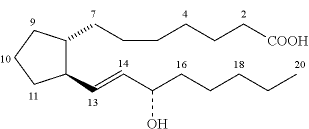 [SMILES: CCCCC[C@H](O)/C=C/[C@H]1CCC[C@@H]1CCCCCCC(=O)O]

The upper side chain (or “alpha chain”) generally contains 7 carbon atoms. The lower side chain (or “omega chain”) generally contains 8 carbon atoms. The end of the alpha chain normally is a carboxylic acid moiety. The side chains may contain 1 to 3 double bonds, most frequently 2, the double bonds being situated between carbon atoms 5 and 6 on the alpha chain and between bonds 13 and 14 on the omega chain. The double bond on the alpha chain generally exhibits cis-configuration, whereas the double bond on the omega chain generally exhibits trans-configuration. According to some embodiments, the substituent group on carbon 15 in the omega chain relates to the prostaglandin's maximal biological activity. In naturally occurring prostaglandins this substituent is hydroxyl.

Different classes of prostaglandins are identified by suffixes A, B, C, D, E, F or J depending on the the configuration and substituents of the five-membered cyclopentane ring. Prostaglandins A, B and C probably are not naturally occurring but rather are artificial prostaglandins; nevertheless, they exert considerable biologic activity.

The configuration of and functionalities attached to the cyclopentane ring are important for selectivity to different prostaglandin receptors. The various configurations include:

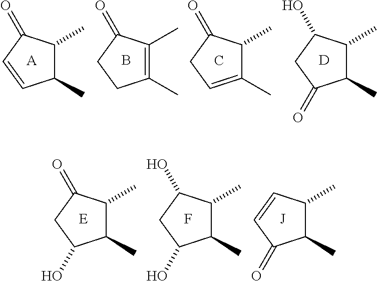

Structures of exemplary prostaglandins are presented below. Where applicable, a hashed line represents a substituent below this paper's plane, a bold wedge represents a substituent above this paper's plane; and a dashed line represent a single or double bond which can be in the cis or trans configuration

Prostaglandin A

The chemical structure of prostaglandin A2 is shown below:

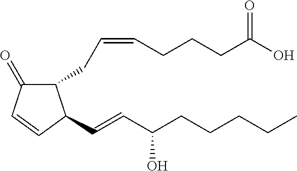 [SMILES: CCCCC[C@H](O)/C=C/[C@H]1C=CC(=O)[C@@H]1C/C=C\CCCC(=O)O]

Prostaglandin B

The chemical structure of prostaglandin B2 is shown below:

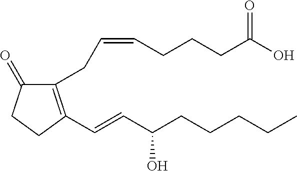 [SMILES: CCCCC[C@H](O)/C=C/C1=C(C/C=C\CCCC(=O)O)C(=O)CC1]

Prostaglandin D

The chemical structure of prostaglandin D2 is shown below:

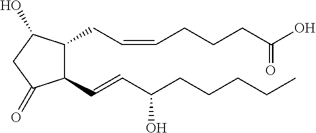 [SMILES: CCCCC[C@H](O)/C=C/[C@H]1C(=O)C[C@H](O)[C@@H]1C/C=C\CCCC(=O)O]

Prostaglandin E

The chemical structure of prostaglandin E₁ (11α,13E,15S)-11,15-dihydroxy-9-oxoprosta-13-en-1-oic acid) (Alprostadil) is shown below:

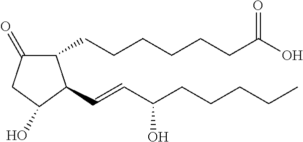 [SMILES: CCCCC[C@H](O)/C=C/[C@H]1[C@H](O)CC(=O)[C@@H]1CCCCCCC(=O)O]

The chemical structure of prostaglandin E2 (9-oxo-11α,15S-dihydroxy-prosta-5Z,13E-dien-1-oic acid) (Dinoprostone) is shown below:

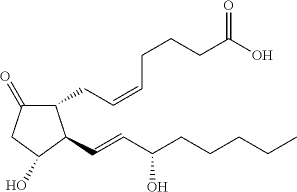 [SMILES: CCCCC[C@H](O)/C=C/[C@H]1[C@H](O)CC(=O)[C@@H]1C/C=C\CCCC(=O)O]

Prostaglandin F

The general chemical structure of prostaglandin F_{2α} is shown below wherein a hashed line represents a substituent below this paper's plane; wherein a bold wedge represents a substituent above this paper's plane; and wherein the dashed lines represent a single or double bond which can be in the cis or trans configuration:

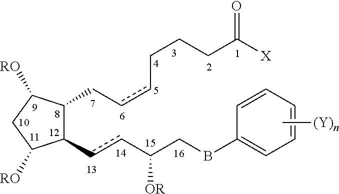

There are several commercially available prostaglandin F analogs. For example, latanoprost [(1R, 2R, 3R, 5S)3, 5-dihydroxy-2-[(3R)-3-hydroxy-5-phenylpentyl]cyclopentyl]-5-heptenoate], marketed by Pfizer as Xalatan®, is a prostaglandin analog in which R is H, B is —CH₂—, n is 0, X is OCH(CH₃)₂, and the dashed lines represent a double bond.

Bimatoprost (cyclopentane N-ethyl heptenamide-5-cis-2-(3α-hydroxy-5-phenyl-1-trans-pentenyl)-3, 5-dihydroxy, [1α, 2β, 3, 3α, 5α], sold by Allergan, Inc. of Irvine, Calif. as Lumigan®, is a 0.03% ophthalmic solution for treating glaucoma. Bimatoprost is a prostaglandin analog in which R is H, B is —CH₂—, n is 0, X is NHC₂H₅ and the dashed lines represent a double bond.

Isopropyl (Z)-7-[(1-R ,2-R ,3-R ,5-S)-3,5-dihydroxy-2-[(1 E ,3 R)-3-hydroxy-4-[(α,α,α-trifluoro-m-tolyl)oxy]-1-butenyl]cyclopentyl]-5-heptenoate, or Travaprost (TRAVATAN® Alcon), another synthetic prostaglandin analog used for treatment of glaucoma, is available as a 0.004% ophthalmic solution. Travoprost is a prostaglandin analog in which R is H, B is 0, Y is CF₃, X is OCH(CH₃)₂.

Prostaglandin H

The chemical structure of prostaglandin H2 is shown below:

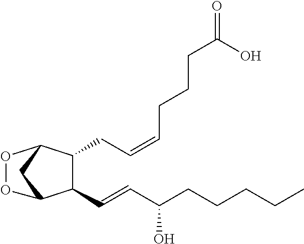

Prostaglandin J

The chemical structure of prostaglandin J2 is shown below:

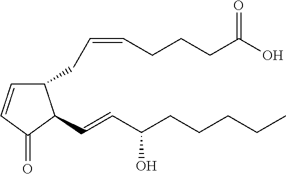 [SMILES: CCCCC[C@H](O)/C=C/[C@H]1C(=O)C=C[C@@H]1C/C=C\CCCC(=O)O]

Prostacyclin

The chemical structure of prostacyclin ((Z)-5-((3aR,4R,5R,6aS)-5-hydroxy-4-((S,E)-3-hydroxyoct-1-enyl)hexahydro-2H-cyclopenta[b]furan-2-ylidene)pentanoic acid) (PGI₂) is shown below:

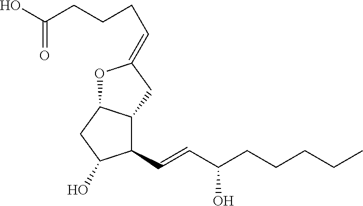 [SMILES: CCCCC[C@H](O)/C=C/[C@@H]1[C@H]2C/C(=C/CCCC(=O)O)O[C@H]2C[C@H]1O]

### Rho Kinase Inhibitors

### Rho Associated Coiled Coil Kinase (ROCK) Proteins

ROCK proteins belong to the protein kinase A, G, and C family (AGC family) of classical serine/threonine protein kinases, a group that also includes other regulators of cell shape and motility, such as citron Rho-interacting kinase (CRIK), dystrophia myotonica protein kinase (DMPK), and the myotonic dystrophy kinase-related Cdc42-binding kinases (MRCKs). The main function of ROCK signaling is regulation of the cytoskeleton through the phosphorylation of downstream substrates, leading to increased actin filament stabilization and generation of actin-myosin contractility. (Morgan-Fisher et al., “Regulation of ROCK Activity in Cancer” (2013) 61:185-198, at 185).

Two homologous mammalian serine/threonine kinases, Rho-associated protein kinases I and II (ROCK I and II), are key regulators of the actin cytoskeleton acting downstream of the small GTPase Rho. ROCK I (alternatively called ROK β) and ROCK II (also known as Rho kinase or ROK α) are 160-kDa proteins encoded by distinct genes. The mRNA of both kinases is ubiquitously expressed, but ROCK I protein is mainly found in organs such as liver, kidney, and lung, whereas ROCK II protein is mainly expressed in muscle and brain tissue. The two kinases have the same overall domain structure and have 64% overall identity in humans, with 89% identity in the catalytic kinase domain. Both kinases contain a coiled-coil region (55% identity) containing a Rho-binding domain (RBD) and a pleckstrin homology (PH) domain split by a C1 conserved region (80% identity). Despite a high degree of homology between the two ROCKs, as well as the fact that they share several common substrates, studies have shown that the two ROCK isoforms also have distinct and non-redundant functions. For example, ROCK I has been shown to be essential for the formation of stress fibers and focal adhesions, whereas ROCK II is required for myosin II-dependent phagocytosis.

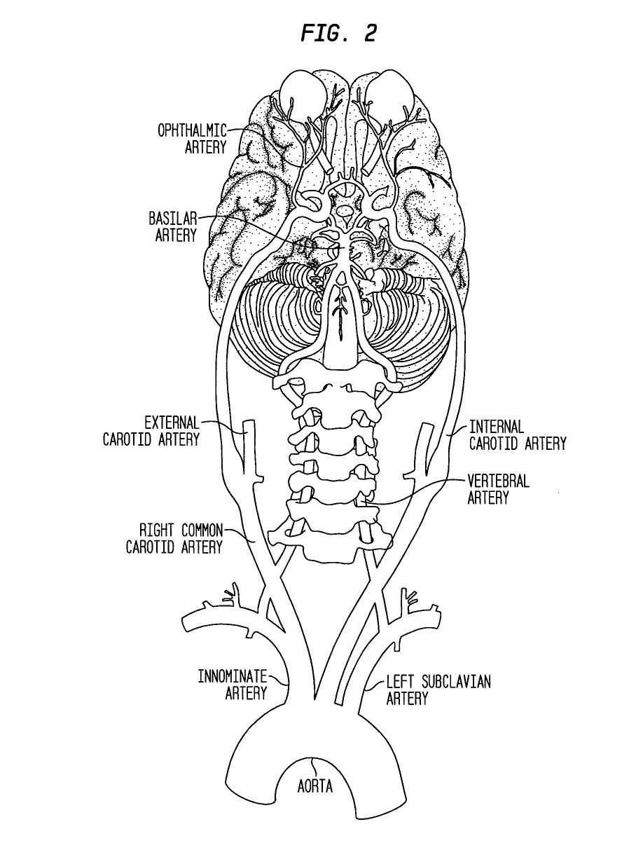

ROCKs exist in a closed, inactive conformation under quiescent conditions, which is changed to an open, active conformation by the direct binding of guanosine triphosphate (GTP)-loaded Rho. (Morgan-Fisher et al., “Regulation of ROCK Activity in Cancer” (2013) 61:185-198). Rho is a small GTPase which functions as a molecular switch, cycling between guanosine diphosphate (GDP) and guanosine triphosphate (GTP) bound states under signaling through growth factors or cell adhesion receptors. (Morgan-Fisher et al., “Regulation of ROCK Activity in Cancer” (2013) 61:185-198, at 185) GTPases are hydrolase enzymes that bind and hydrolyse GTP. In a similar way to ATP, GTP can act as an energy carrier, but it also has an active role in signal transduction, particularly in the regulation of G protein activity. G proteins, including Rho GTPases, cycle between an inactive GDP-bound and an active GTP-bound conformation. (FIG. 2) The transition between the two conformational states occurs through two distinct mechanisms: activation by GTP loading and inactivation by GTP hydrolysis. GTP loading is a two-step process that requires the release of bound GDP and its replacement by a GTP molecule. Nucleotide release is a spontaneous but slow process that has to be catalyzed by RHO-specific guanine nucleotide exchange factors (RHOGEFs), which associate with RHO GTPases and trigger release of the nucleotide. The resulting nucleotide-free binary complex has no particular nucleotide specificity. However, the cellular concentration of GTP is markedly higher than that of GDP, which favors GTP loading, resulting in the activation of RHO GTPases.

Conversely, to turn off the switch, GTP has to be hydrolyzed. This is facilitated by RHO-specific GTPase-activating proteins (RHOGAPs), which stimulate the intrinsically slow hydrolytic activity of RHO proteins. Although guanine nucleotide exchange factors (GEFs) and GTPase-activating proteins (GAPs) are the canonical regulators of this cycle, several alternative mechanisms, such as post-translational modifications, may fine-tune the RHO switch. In addition, inactive RHO GTPases are extracted by RHO-specific guanine nucleotide dissociation inhibitors (RHOGDIs) from cell membranes to prevent their inappropriate activation and to protect them from misfolding and degradation. (R. Garcia-Mata et al. Nature Reviews Molecular Cell Biology (2011) 12:493-504; at 494)

Many proteins aid in activating and inhibiting ROCK I and ROCK II. For example, small GTP-binding proteins RhoA (which controls cell adhesion and motility through organization of the actin cytoskeleton and regulation of actomyosin contractility (Yoshioka, K. et al., “Overexpression of Small GTP-binding protein RhoA promotes Invasion of Tumor Cells,” J. Cancer Res. (1999) 59: 2004-2010, RhoB (which is localized primarily on endosomes, has been shown to regulate cytokine trafficking and cell survival) and RhoC (which may be more important in cell locomotion) (Wheeler, A P, Ridley, A J, “Why three Rho proteins? RhoA, RhoB , RhoC and cell motility,” Exp. Cell Res. 2004) 301(1): 43-49) associate with and activate the ROCK proteins. Other GTP binding proteins, such as RhoE, Ras associated with diabetes (Rad), and Gem (a member of the RGK family of GTP-binding proteins within the Ras superfamily possessing a ras-like core and terminal extensions whose expression inhibited ROK beta-mediated phosphorylation of myosin light chain and myosin phosphatase, but not LIM kinase, see Ward Y., et al., J. Cell Biol. 157(2): 291-302 (2002)), inhibit ROCK, binding at sites distinct from the canonical Ras binding domain (RBD). Association with the PDK1 kinase promotes ROCK I activity by blocking RhoE association.

ROCK activation leads to a concerted series of events that promote force generation and morphological changes. These events contribute directly to a number of actin-myosin mediated processes, such as cell motility, adhesion, smooth muscle contraction, neurite retraction and phagocytosis. In addition, ROCK kinases play roles in proliferation, differentiation, apoptosis and oncogenic transformation, although these responses can be cell type-dependent. (Olson (2008) “Applications for ROCK kinase inhibition” Curr Opin Cell Biol 20(2): 242-248, at 242-243).

ROCK I and ROCK II promote actin-myosin mediated contractile force generation through the phosphorylation of numerous downstream target proteins, including ezrin/radixin/moesin (ERM), the LIM-kinases (LIMK), myosin light chain (MLC), and MLOC phosphatase (MLCP). ROCK phosphorylates LIM kinases-1 and -2 (LIMK1 and LIMK2) at conserved Threonines in their activation loops, increasing LIMK activity and the subsequent phosphorylation of cofilin proteins, which blocks their F-actin-severing activity. ROCK also directly phosphorylates the myosin regulatory light chain, myosin light chain II (MLC), and the myosin binding subunit (MYPT1) of the MLC phosphatase to inhibit catalytic activity. Many of these effects are also amplified by ROCK-mediated phosphorylation and activation of the Zipper-interacting protein kinase (ZIPK), a serine/threonine kinase which is involved in the regulation of apoptosis, autophagy, transcription, translation, actin cytoskeleton reorganization, cell motility, smooth muscle contraction and mitosis, which phosphorylates many of the same substrates as ROCK.

The phosphorylation of MLC by ROCK provides the chemical energy for actin-myosin ratcheting, and also phosphorylates myosin light chain phosphatase (MLCP), thereby inactivating MLCP and preventing its dephosphorylation of MLC. Thus, ROCK promotes actin-myosin movement by activation and stabilization. Other known substrates of ROCK include the cytoskeleton related proteins such as the ERM proteins, and focal adhesion kinase (FAK). The ERM proteins function to connect transmembrane proteins to the cytoskeleton. (Street and Bryan (2011) “Rho Kinase Proteins-Pleiotropic Modulators of Cell Survival and Apoptosis” Anticancer Res. November 31(11): 3645-3657, at 3650).

ROCK has been Linked to Apoptosis, Cell Survival, and Cell Cycle Progression

Rho-ROCK signaling has been implicated in cell cycle regulation. Rho-ROCK signaling increases cyclin D1 and Cip1 protein levels, which stimulate G1/S cell cycle progression. (Morgan-Fisher et al., “Regulation of ROCK Activity in Cancer” (2013) 61:185-198, at 189). Polyploidization naturally occurs in megakaryocytes due to an incomplete mitosis, which is related to a partial defect in Rho-ROCK activation, and leads to an abnormal contractile ring lacking myosin IIA.

Rho-ROCK signaling also has been linked to apoptosis and cell survival. During apoptosis, ROCK I and ROCK II are altered to become constitutively-active kinases. Through proteolytic cleavage by caspases (ROCK I) or granzyme B (ROCK II), a carboxyl-terminal portion is removed that normally represses activity. Interaction with phosphatidyl inositol (3,4,5)-triphosphate (PIP3) provides an additional regulatory mechanism by localizing ROCK II to the plasma membrane where it can undertake spatially restricted activities, i.e. the regulation by localization of enzymatic activity. Phosphorylation at multiple specific sites by polo-like kinase 1 was found to promote ROCK II activation by RhoA. (Olson (2008) “Applications for ROCK kinase inhibition” Curr Opin Cell Biol 20(2): 242-248, at 242.) Additional Serine/Threonine and Tyrosine kinases may also regulate ROCK activity given that more phosphorylations have been identified. (Olson (2008) “Applications for ROCK kinase inhibition” Curr Opin Cell Biol 20(2): 242-248, at 242.) Specifically, protein oligomerization induces N-terminal trans-phosphorylation. (K. Riento and A. J. Ridley, “ROCKs: multifunction kinases in cell behavior.” Nat Rev Mol Cell Biol (2003) 4:446-456). Other direct activators include intracellular second messengers such as arachodonic acid and sphingosylphosphorylcholine which can activate ROCK independently of Rho. Furthermore, ROCK1 activity can be induced during apoptosis. (Mueller, B. K. et al., “Rho Kinase, a promising drug target for neurological disorders.” (2005) Nat Rev Mol Cell Biol 4(6): 387-398.)

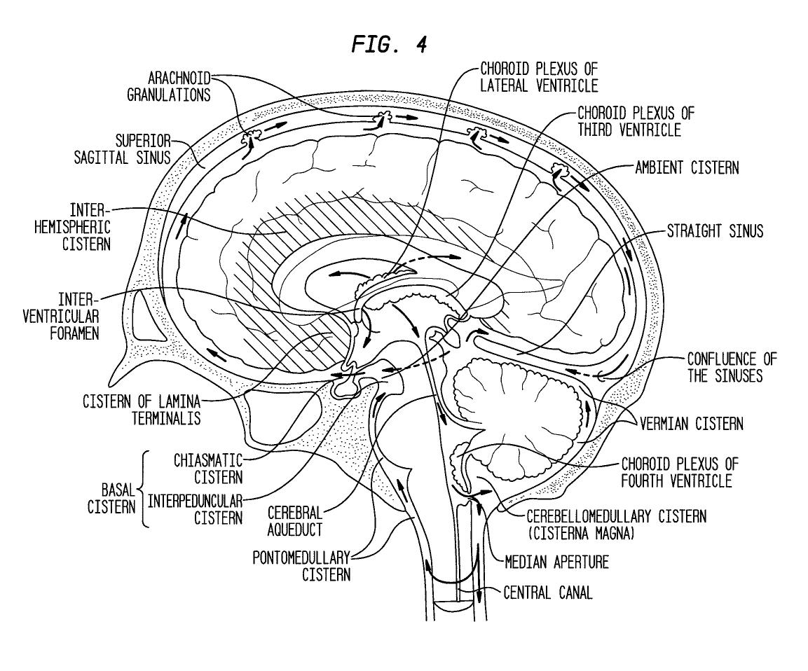

ROCK protein signaling reportedly acts in either a pro- or anti-apoptotic fashion depending on cell type, cell context and microenvironment. For instance, ROCK proteins are essential for multiple aspects of both the intrinsic and extrinsic apoptotic processes, including regulation of cytoskeletal-mediated cell contraction and membrane blebbing, nuclear membrane disintegration, modulation of Bc12-family member and caspase expression/activation and phagocytosis of the fragmented apoptotic bodies (FIG. 4) (B. K. Mueller et al. “Rho Kinase, a promising drug target for neurological disorders.” (2005) Nature Reviews: Drug Discovery 4:387-398). In contrast, ROCK signaling also exhibits pro-survival roles. Though a wealth of data exists to suggest both pro- and anti-survival roles for ROCK proteins, the molecular mechanisms that modulate these pleitropic roles are largely unknown. (C. A. Street and B. A. Bryan, “Rho Kinase proteins—pleiotropic modulators of cell survival and apoptosis.” (2011) Anticancer Res. 31(11):3645-3657; FIG. 4.)

The importance of the cytoskeleton for various cellular functions, combined with the pleiotropy of ROCK targeted phosphorylation, accounts for the wide range of animal models in which ROCK inhibitors, such as Y-27632, have shown beneficial effects. These include experimental asthma, Alzheimer's disease, Parkinson's disease, systemic lupus erythematosis, cardiovascular disease, organ transplant, diabetes, and erectile dysfunction, among others. (Olson (2008) “Applications for ROCK kinase inhibition” Curr Opin Cell Biol 20(2): 242-248).

Data from ROCK I knockout mice supports their use to treat cardiovascular diseases. Using a variety of models that mimic chronic high blood pressure, partial or full deletion of ROCK I reduced cardiac fibrosis without affective cardiomyocyte hypertrophy. In addition, pressure overload was less effective at inducing cardiomyocyte apoptosis in ROCK I-/-mice relative to controls, suggesting a role for ROCK I in myocardial failure. (Olson (2008) “Applications for ROCK kinase inhibition” Curr Opin Cell Biol 20(2): 242-248, at 243-244.)

Exemplary Rho kinase inhibitors include, without limitation, Y-27632 2HCl (R&D Systems Inc., Minneapolis, Minn.), Triazovivin® (StemRD, Burlingame, Calif.), Slx-2119 (MedChem Express, Namiki Shoji Cop., LTD), WF-536 [(+)-®-4-(1-aminoethyl)-N-(4-pyridyl) benzamide monohydrochloride] (Mitsubishi Pharma Corporation, Osaka, Japan), RK1-1447 (University of South Florida, Tampa, Fla., and Moffitt Cancer Center, Tampa, Fla.; Roberta Pireddu et al., “Pyridylthiazole-based ureas as inhibitors of Rho associated protein kinases (ROCK1 and 2).” (2012) Medchemcomm. 3(6):699-709), Fasudil® (Asahi-KASEI Corp., Osaka, Japan), Fasudil® hydrochloride (R&D Systems Inc., Minneapolis, Minn.), GSK429286A (R&D Systems Inc., Minneapolis, Minn.), Rockout® (EMD Millipore, Philadelphia, Pa.), SR 3677 dihydrochloride (R&D Systems Inc., Minneapolis, Minn.); SB 772077B (R&D Systems Inc., Minneapolis, Minn.), AS 1892802 (R&D Systems Inc., Minneapolis, Minn.), H 1152 dihydrochloride (R&D Systems Inc., Minneapolis, Minn.), GSK 269962 (R&D Systems Inc., Minneapolis, Minn.), HA 1100 hydrochloride (R&D Systems Inc., Minneapolis, Minn.), Glycyl-H-1152 dihydrochloride (R&D Systems Inc., Minneapolis, Minn.), AR-12286 (Aerie Pharmaceuticals), AR-13324 (Rhopressa, Aerie Pharmaceuticals), AMA-0076 (Amakem Therapeutics), and K-115 (Kumatomo University, Japan). According to some other embodiments, the additional therapeutic agent includes a combination of a Rho kinase inhibitor and a prostaglandin analog.

### Pharmaceutically Acceptable Carrier

According to some embodiments, the pharmaceutical composition does not comprise a pharmaceutically acceptable carrier.

According to one embodiment, the pharmaceutically acceptable carrier is a solid carrier or excipient. According to another embodiment, the pharmaceutically acceptable carrier is a gel-phase carrier or excipient. Examples of carriers or excipients include, but are not limited to, calcium carbonate, calcium phosphate, various monomeric and polymeric sugars (including without limitation hyaluronic acid), starches, cellulose derivatives, gelatin, and polymers. An exemplary carrier can also include saline vehicle, e.g. hydroxyl propyl methyl cellulose (HPMC) in phosphate buffered saline (PBS).

According to some embodiments, the pharmaceutically acceptable carrier imparts stickiness to the composition. According to one embodiment, the pharmaceutically acceptable carrier comprises hyaluronic acid. According to some embodiments, the pharmaceutically acceptable carrier comprises 0% to 5% hyaluronic acid. According to one embodiment, the pharmaceutically acceptable carrier comprises less than 0.05% hyaluronic acid. According to another embodiment, the pharmaceutically acceptable carrier comprises less than 0.1% hyaluronic acid. According to another embodiment, the pharmaceutically acceptable carrier comprises less than 0.2% hyaluronic acid. According to another embodiment, the pharmaceutically acceptable carrier comprises less than 0.3% hyaluronic acid. According to another embodiment, the pharmaceutically acceptable carrier comprises less than 0.4% hyaluronic acid. According to another embodiment, the pharmaceutically acceptable carrier comprises less than 0.5% hyaluronic acid. According to another embodiment, the pharmaceutically acceptable carrier comprises less than 0.6% hyaluronic acid. According to another embodiment, the pharmaceutically acceptable carrier comprises less than 0.7% hyaluronic acid. According to another embodiment, the pharmaceutically acceptable carrier comprises less than 0.8% hyaluronic acid. According to another embodiment, the pharmaceutically acceptable carrier comprises less than 0.9% hyaluronic acid. According to another embodiment, the pharmaceutically acceptable carrier comprises less than 1.0% hyaluronic acid. According to another embodiment, the pharmaceutically acceptable carrier comprises less than 1.1% hyaluronic acid. According to another embodiment, the pharmaceutically acceptable carrier comprises less than 1.2% hyaluronic acid. According to another embodiment, the pharmaceutically acceptable carrier comprises less than 1.3% hyaluronic acid. According to another embodiment, the pharmaceutically acceptable carrier comprises less than 1.4% hyaluronic acid. According to another embodiment, the pharmaceutically acceptable carrier comprises less than 1.5% hyaluronic acid. According to another embodiment, the pharmaceutically acceptable carrier comprises less than 1.6% hyaluronic acid. According to another embodiment, the pharmaceutically acceptable carrier comprises less than 1.7% hyaluronic acid. According to another embodiment, the pharmaceutically acceptable carrier comprises less than 1.8% hyaluronic acid. According to another embodiment, the pharmaceutically acceptable carrier comprises less than 1.9% hyaluronic acid. According to another embodiment, the pharmaceutically acceptable carrier comprises less than 2.0% hyaluronic acid. According to another embodiment, the pharmaceutically acceptable carrier comprises less than 2.1% hyaluronic acid. According to another embodiment, the pharmaceutically acceptable carrier comprises less than 2.2% hyaluronic acid. According to another embodiment, the pharmaceutically acceptable carrier comprises less than 2.3% hyaluronic acid. According to another embodiment, the pharmaceutically acceptable carrier comprises less than 2.4% hyaluronic acid. According to another embodiment, the pharmaceutically acceptable carrier comprises less than 2.5% hyaluronic acid. According to another embodiment, the pharmaceutically acceptable carrier comprises less than 2.6% hyaluronic acid. According to another embodiment, the pharmaceutically acceptable carrier comprises less than 2.7% hyaluronic acid. According to another embodiment, the pharmaceutically acceptable carrier comprises less than 2.8% hyaluronic acid. According to another embodiment, the pharmaceutically acceptable carrier comprises less than 2.9% hyaluronic acid. According to another embodiment, the pharmaceutically acceptable carrier comprises less than 3.0% hyaluronic acid. According to another embodiment, the pharmaceutically acceptable carrier comprises less than 3.5% hyaluronic acid. According to another embodiment, the pharmaceutically acceptable carrier comprises less than 4.0% hyaluronic acid. According to another embodiment, the pharmaceutically acceptable carrier comprises less than 4.5% hyaluronic acid. According to another embodiment, the pharmaceutically acceptable carrier comprises less than 5.0% hyaluronic acid.

According to some embodiments, the pharmaceutically acceptable carrier includes, but is not limited to, a gel, slow-release solid or semisolid compound, optionally as a sustained release gel. According to some embodiments, the pharmaceutical carrier is a polymer. According to some embodiments, the polymer is a slow release polymer. According to some embodiments, the polymer is poly (D, L-Lactide-co-glycolide). According to some embodiments, the polymer is poly(orthoester). According to some embodiments, the polymer is poly(anhydride). According to some embodiments, the polymer is polylactide-polyglycolide.

According to some such embodiments, the therapeutic agent is embedded into the pharmaceutically acceptable carrier. According to some embodiments, the therapeutic agent is coated on the pharmaceutically acceptable carrier. The coating can be of any desired material, preferably a polymer or mixture of different polymers. Optionally, the polymer can be utilized during the granulation stage to form a matrix with the active ingredient so as to obtain a desired release pattern of the active ingredient. The gel, slow-release solid or semisolid compound is capable of releasing the active agent over a desired period of time. The gel, slow-release solid or semisolid compound can be implanted in a tissue within human brain, for example, but not limited to, in close proximity to a blood vessel, such as a cerebral artery.

According to another embodiment, the pharmaceutically acceptable carrier comprises a slow-release solid compound. According to one such embodiment, the therapeutic agent is embedded in the slow-release solid compound or coated on the slow-release solid compound. According to yet another embodiment, the pharmaceutically acceptable carrier comprises a slow-release microparticle containing the therapeutic agent.

According to another embodiment, the pharmaceutically acceptable carrier is a gel compound, such as a biodegradable hydrogel.

According to some embodiments, the pharmaceutically acceptable carrier comprises a SABER™ formulation. SABER™ formulations comprise a drug and a high viscosity liquid carrier material (HVLCM), meaning nonpolymeric, nonwater soluble liquids with a viscosity of at least 5,000 cP at 37° C. that do not crystallize neat under ambient or physiological conditions. HVLCMs may be carbohydrate-based, and may include one or more cyclic carbohydrates chemically combined with one or more carboxylic acids, such as sucrose acetate isobutyrate (SAIB). HVLCMs also include nonpolymeric esters or mixed esters of one or more carboxylic acids, having a viscosity of at least 5,000 cP at 37° C., that do not crystallize neat under ambient or physiological conditions, wherein when the ester contains an alcohol moiety (e.g., glycerol). The ester may, for example comprise from about 2 to about 20 hydroxy acid moieties.

Additional components can include, without limitation, a rheology modifier, and/or a network former. A rheology modifier is a substance that possesses both a hydrophobic and a hydrophilic moiety used to modify viscosity and flow of a liquid formulation, for example, caprylic/capric triglyceride (Migliol 810), isopropyl myristate (IM or IPM), ethyl oleate, triethyl citrate, dimethyl phthalate, and benzyl benzoate. A network former is a compound that forms a network structure when introduced into a liquid medium. Exemplary network formers include cellulose acetate butyrate, carbohydrate polymers, organic acids of carbohydrate polymers and other polymers, hydrogels, as well as particles such as silicon dioxide, ion exchange resins, and/or fiberglass, that are capable of associating, aligning or congealing to form three dimensional networks in an aqueous environment.

### Particulate Formulation

According to some embodiments, the therapeutic agent is provided in the form of a particle. The term “particle” as used herein refers to nanoparticles or microparticles (or in some instances smaller or larger) that may contain in whole or in part the calcium channel antagonist. According to some embodiments, the particulate formulation comprises a plurality of particles impregnated with the therapeutic agent. According to one embodiment, the therapeutic agent is contained within the core of the particle surrounded by a coating. According to another embodiment, the therapeutic agent is dispersed throughout the surface of the particle. According to another embodiment, the therapeutic agent is disposed on or in the particle. According to another embodiment, the therapeutic agent is disposed throughout the surface of the particle. According to another embodiment, the therapeutic agent is adsorbed into the particle.

According to some such embodiments, the particles are of uniform size distribution. According to some embodiments, the uniform distribution of particle size is achieved by a homogenization process to form a uniform emulsion comprising the particles. According to some such embodiments, each particle comprises a matrix. According to some embodiments, the matrix comprises the therapeutic agent(s).

According to some embodiments, the pharmaceutical composition is flowable. According to some embodiments, the particulate formulation component of the pharmaceutical composition is flowable.

According to some embodiments, the particle is selected from the group consisting of a zero order release, first order release, second order release, delayed release, sustained release, immediate release, and a combination thereof. The particle can include, in addition to therapeutic agent(s), any of those materials routinely used in the art of pharmacy and medicine, including, but not limited to, erodible, nonerodible, biodegradable, or nonbiodegradable material or combinations thereof.

According to some embodiments, the particle contains the therapeutic agent in a solution or in a semi-solid state. According to some embodiments, the particle is a microparticle that contains the therapeutic agent, in whole or in part. According to some embodiments, the particle is a nanoparticle that contains the therapeutic agent, in whole or in part. According to some embodiments, the particles can be of virtually any shape.

According to some embodiments, the particle size is at least 50 nm. According to some embodiments, the particle size is at least 100 nm. According to some embodiments, the particle size is at least 500 nm. According to some embodiments, the particle size is at least about 1 μm. According to some embodiments, the particle size is at least about 5 μm. According to some embodiments, the particle size is at least about 10 μm. According to some embodiments, the particle size is at least about 15 μm. According to some embodiments, the particle size is at least about 20 μm. According to one embodiment, the particle size is at least about 25 μm. According to another embodiment, the particle size is at least about 30 μm. According to another embodiment, the particle size is at least about 35 μm. According to another embodiment, the particle size is at least about 40 μm. According to another embodiment, the particle size is at least about 45 μm. According to another embodiment, the particle size is at least about 50 μm. According to another embodiment, the particle size is at least about 55 μm. According to another embodiment, the particle size is at least about 60 μm. According to another embodiment, the particle size is at least about 65 μm. According to another embodiment, the particle size is at least about 70 μm. According to another embodiment, the particle size is at least about 75 μm. According to another embodiment, the particle size is at least about 80 μm. According to another embodiment, the particle size is at least about 85 μm. According to another embodiment, the particle size is at least about 90 μm. According to another embodiment, the particle size is at least about 95 μm. According to another embodiment, the particle size is at least about 100 μm.

According to another embodiment, the therapeutic agent can be provided in form of a filament, string, cord or thread. The term “filament” as used herein refers to a very fine thread or threadlike structure, fiber or fibril. The term “string” as used herein refers to a slender cord or thread. The term “cord” as used herein, refers to a structure made of several strands braided, twisted, or woven together. The term “thread” as used herein refers to a cord of a material composed of two or more filaments twisted together. The filament, string, cord or thread can contain the therapeutic agent in a core surrounded by a coating, or the therapeutic agent can be dispersed throughout the filament, string, cord or thread, or the therapeutic agent(s) may be absorbed into the filament, string, cord or thread. The filament, string, cord or thread can be of any order release kinetics, including zero order release, first order release, second order release, delayed release, sustained release, immediate release, etc., and any combination thereof. The filament, string, cord or thread can include, in addition to the therapeutic agent(s), any of those materials routinely used in the art of pharmacy and medicine, including, but not limited to, erodible, nonerodible, biodegradable, or nonbiodegradable material or combinations thereof.

According to another embodiment, the therapeutic agent can be provided in at least one film or sheet. The term “film” as used herein refers to a thin skin or membrane. The term “sheet” as used herein, refers to a broad, relatively thin, form, piece or material. The film or sheet can contain the therapeutic agent and optionally an additional therapeutic agent in a core surrounded by a coating, or the therapeutic agent and optionally an additional therapeutic agent can be dispersed throughout the film or sheet, or the therapeutic agent can be absorbed into the film or sheet. The film or sheet can be of any order release kinetics, including zero order release, first order release, second order release, delayed release, sustained release, immediate release, etc., and any combination thereof. The film or sheet can include, in addition to the therapeutic agent and optionally an additional therapeutic agent, any of those materials routinely used in the art of pharmacy and medicine, including, but not limited to, erodible, nonerodible, biodegradable, or nonbiodegradable material or combinations thereof.

According to some embodiments, the pharmaceutical composition further comprises a preservative agent. According to some such embodiments, the pharmaceutical composition is presented in a unit dosage form. Exemplary unit dosage forms include, but are not limited to, ampoules or multi-dose containers.

According to some embodiments, the microparticulate formulation comprises a suspension of microparticles. According to some embodiments, the pharmaceutical composition further comprises at least one of a suspending agent, a stabilizing agent and a dispersing agent. According to some such embodiments, the pharmaceutical composition is presented as a suspension. According to some such embodiments, the pharmaceutical composition is presented as a solution. According to some such embodiments, the pharmaceutical composition is presented as an emulsion.

According to some embodiments, a formulation of the pharmaceutical composition comprises an aqueous solution of the therapeutic agent in water-soluble form. According to some embodiments, the formulation of the pharmaceutical composition comprises an oily suspension of the therapeutic agent. An oily suspension of the therapeutic agent can be prepared using suitable lipophilic solvents. Exemplary lipophilic solvents or vehicles include, but are not limited to, fatty oils such as sesame oil, or synthetic fatty acid esters, such as ethyl oleate or triglycerides. According to some embodiments, the formulation of the pharmaceutical composition comprises an aqueous suspension of the therapeutic agent. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions. Alternatively, the therapeutic agent can be in powder form for constitution with a suitable vehicle, e.g., sterile pyrogen-free water, before use.

Suitable liquid or solid pharmaceutical preparations include, for example, microencapsulated dosage forms, and if appropriate, with one or more excipients, encochleated, coated onto microscopic gold particles, contained in liposomes, pellets for implantation into the tissue, or dried onto an object to be rubbed into the tissue. As used herein, the term “microencapsulation” refers to a process in which very tiny droplets or particles are surrounded or coated with a continuous film of biocompatible, biodegradable, polymeric or non-polymeric material to produce solid structures including, but not limited to, nonpareils, pellets, crystals, agglomerates, microspheres, or nanoparticles. Such pharmaceutical compositions also can be in the form of granules, beads, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, or solubilizers are customarily used as described above. The pharmaceutical compositions are suitable for use in a variety of drug delivery systems. For a brief review of methods for drug delivery, see Langer (1990) Science 249, 1527-1533, which is incorporated herein by reference.

### Microencapsulation Process

Examples of microencapsulation processes and products; methods for the production of emulsion-based microparticles; emulsion-based microparticles and methods for the production thereof; solvent extraction microencapsulation with tunable extraction rates; microencapsulation process with solvent and salt; a continuous double emulsion process for making microparticles; drying methods for tuning microparticle properties, controlled release systems from polymer blends; polymer mixtures comprising polymers having different non-repeating units and methods for making and using the same; and an emulsion based process for preparing microparticles and workhead assembly for use with same are disclosed and described in U.S. Pat. No. 5, 407,609 (entitled Microencapsulation Process and Products Thereof), U.S. application Ser. No. 10/553,003 (entitled Method for the production of emulsion-based microparticles), U.S. application Ser. No. 11/799,700 (entitled Emulsion-based microparticles and methods for the production thereof), U.S. application Ser. No. 12/557,946 (entitled Solvent Extraction Microencapsulation With Tunable Extraction Rates) , U.S. application Ser. No. 12/779,138 (entitled Hyaluronic Acid (HA) Injection Vehicle), U.S. application Ser. No. 12/562,455 entitled Microencapsulation Process With Solvent And Salt) , U.S. application Ser. No. 12/338,488 (entitled Process For Preparing Microparticles Having A Low Residual Solvent Volume); U.S. application Ser. No. 12/692,027 (entitled Controlled Release Systems From Polymer Blends); U.S. application Ser. No. 12/692,020 (entitled Polymer Mixtures Comprising Polymers Having Different Non-Repeating Units And Methods For Making And Using Same); U.S. application Ser. No. 10/565,401 (entitled “Controlled release compositions”); U.S. application Ser. No. 12/692,029 (entitled “Drying Methods for Tuning Microparticle Properties); U.S. application Ser. No. 12/968,708 (entitled “Emulsion Based Process for Preparing Microparticles and Workhead for Use with Same); and U.S. application Ser. No. 13/074,542 (entitled Composition and Methods for Improved Retention of a Pharmaceutical Composition at a Local Administration Site”) The contents of each of these are incorporated herein by reference in its entirety.

According to some embodiments, delivery of the therapeutic agent using microparticle technology involves bioresorbable, polymeric particles that encapsulate the therapeutic agent and the optional additional therapeutic agent.

According to one embodiment, the microparticle formulation comprises a polymer matrix, wherein the therapeutic agent is impregnated in the polymer matrix. According to one embodiment, the polymer is a slow release polymer. According to one embodiment, the polymer is poly (D, L-Lactide-co-glycolide). According to another embodiment, the polymer is poly(orthoester). According to another embodiment, the polymer is poly(anhydride). According to another embodiment, the polymer is polylactide-polyglycolide.

Both non-biodegradable and biodegradable polymeric materials can be used in the manufacture of particles for delivering the therapeutic agent. Such polymers can be natural or synthetic polymers. The polymer is selected based on the period of time over which release is desired. Bioadhesive polymers of particular interest include, but are not limited to, bioerodible hydrogels as described by Sawhney et al in Macromolecules (1993) 26, 581-587, the teachings of which are incorporated herein. Exemplary bioerodible hydrogels include, but are not limited to, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). According to one embodiment, the bioadhesive polymer is hyaluronic acid. According to some such embodiments, the bioadhesive polymer includes less than about 2.3% of hyaluronic acid.

SABER™ formulations comprise a drug and a high viscosity liquid carrier material (HVLCM), meaning nonpolymeric, nonwater soluble liquids with a viscosity of at least 5,000 cP at 37° C. that do not crystallize neat under ambient or physiological conditions. HVLCMs may be carbohydrate-based, and may include one or more cyclic carbohydrates chemically combined with one or more carboxylic acids, such as sucrose acetate isobutyrate (SAIB). HVLCMs also include nonpolymeric esters or mixed esters of one or more carboxylic acids, having a viscosity of at least 5,000 cP at 37° C., that do not crystallize neat under ambient or physiological conditions, wherein when the ester contains an alcohol moiety (e.g., glycerol). The ester may, for example comprise from about 2 to about 20 hydroxy acid moieties.

Additional components can include, without limitation, a rheology modifier, and/or a network former. A rheology modifier is a substance that possesses both a hydrophobic and a hydrophilic moiety used to modify viscosity and flow of a liquid formulation, for example, caprylic/capric triglyceride (Migliol 810), isopropyl myristate (IM or IPM), ethyl oleate, triethyl citrate, dimethyl phthalate, and benzyl benzoate. A network former is a compound that forms a network structure when introduced into a liquid medium. Exemplary network formers include cellulose acetate butyrate, carbohydrate polymers, organic acids of carbohydrate polymers and other polymers, hydrogels, as well as particles such as silicon dioxide, ion exchange resins, and/or fiberglass,that are capable of associating, aligning or congealing to form three dimensional networks in an aqueous environment.

According to some embodiments, the pharmaceutical composition is formulated for parenteral injection, implantation, topical administration, or a combination thereof. According to some such embodiments, the pharmaceutical composition is in the form of a pharmaceutically acceptable sterile aqueous or nonaqueous solution, dispersion, suspension or emulsion or a sterile powder for reconstitution into a sterile injectable solution or dispersion. Examples of suitable aqueous and nonaqueous carriers, diluents, solvents or vehicles include, but are not limited to, water, ethanol, dichloromethane, acetonitrile, ethyl acetate, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Suspensions can further contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

According to some embodiments, the pharmaceutical composition is formulated in an injectable depot form. Injectable depot forms are made by forming microencapsulated matrices of therapeutic agent in a biodegradable polymer. Depending upon the ratio of drug to polymer and the nature of the particular polymer employed, the rate of drug release may be controlled. Such long acting formulations can be formulated with suitable polymeric or hydrophobic materials (for example as an emulsion in an acceptable oil) or ion exchange resins, or as sparingly soluble derivatives, for example, as a sparingly soluble salt. Examples of biodegradable polymers include, but are not limited to, polylactide-polyglycolide, poly(orthoesters) and poly(anhydrides). Depot injectable formulations are also prepared by entrapping the drug in liposomes or microemulsions which are compatible with body tissues.

According to some embodiments, the therapeutic agent is impregnated in or on a polyglycolide (PGA) matrix. PGA is a linear aliphatic polyester developed for use in sutures. Studies have reported PGA copolymers formed with trimethylene carbonate, polylactic acid (PLA), and other polyesters like polycaprolactone. Some of these copolymers may be formulated as microparticles for sustained drug release.

According to some embodiments, the therapeutic agent is impregnated in or on a polyester—polyethylene glycol matrix. Polyester—polyethylene glycol compounds can be synthesized; these are soft and may be used for drug delivery.

According to some embodiments, the therapeutic agent is impregnated in or on a poly (amino)-derived biopolymer matrix. Poly (amino)-derived biopolymers can include, but are not limited to, those containing lactic acid and lysine as the aliphatic diamine (see, for example, U.S. Pat. No. 5,399,665), and tyrosine-derived polycarbonates and polyacrylates. Modifications of polycarbonates may alter the length of the alkyl chain of the ester (ethyl to octyl), while modifications of polyarylates may further include altering the length of the alkyl chain of the diacid (for example, succinic to sebasic), which allows for a large permutation of polymers and great flexibility in polymer properties.

According to some embodiments, the therapeutic agent is impregnated in or on a polyanhydride matrix. Polyanhydrides are prepared by the dehydration of two diacid molecules by melt polymerization (see, for example, U.S. Pat. No. 4,757,128). These polymers degrade by surface erosion (as compared to polyesters that degrade by bulk erosion). The release of the drug can be controlled by the hydrophilicity of the monomers chosen.

According to some embodiments, the therapeutic agent is impregnated in or on a photopolymerizable biopolymer matrix. Photopolymerizable biopolymers include, but are not limited to, lactic acid/polyethylene glycol/acrylate copolymers.

According to some embodiments, the therapeutic agent is impregnated in or on a hydrogel matrix. The term “hydrogel” refers to a substance resulting in a solid, semisolid, pseudoplastic or plastic structure containing a necessary aqueous component to produce a gelatinous or jelly-like mass. Hydrogels generally comprise a variety of polymers, including hydrophilic polymers, acrylic acid, acrylamide and 2-hydroxyethylmethacrylate (HEMA).

According to some embodiments, the therapeutic agent is impregnated in or on a naturally-occurring biopolymer matrix. Naturally-occurring biopolymers include, but are not limited to, protein polymers, collagen, polysaccharides, and photopolymerizable compounds.

According to some embodiments, the therapeutic agent is impregnated in or on a protein polymer matrix. Protein polymers have been synthesized from self-assembling protein polymers such as, for example, silk fibroin, elastin, collagen, and combinations thereof.

According to some embodiments, the therapeutic agent is impregnated in or on a naturally-occurring polysaccharide matrix. Naturally-occurring polysaccharides include, but are not limited to, chitin and its derivatives, hyaluronic acid, dextran and cellulosics (which generally are not biodegradable without modification), and sucrose acetate isobutyrate (SAIB).

According to some embodiments, the therapeutic agent is impregnated in or on a chitin matrix. Chitin is composed predominantly of 2-acetamido-2-deoxy-D-glucose groups and is found in yeasts, fungi and marine invertebrates (shrimp, crustaceous) where it is a principal component of the exoskeleton. Chitin is not water soluble and the deacetylated chitin, chitosan, only is soluble in acidic solutions (such as, for example, acetic acid). Studies have reported chitin derivatives that are water soluble, very high molecular weight (greater than 2 million daltons), viscoelastic, non-toxic, biocompatible and capable of crosslinking with peroxides, gluteraldehyde, glyoxal and other aldehydes and carbodiamides, to form gels.

According to some embodiments, the therapeutic agent is impregnated in or on a hyaluronic acid (HA) matrix. Hyaluronic acid (HA), which is composed of alternating glucuronidic and glucosaminidic bonds and is found in mammalian vitreous humor, extracellular matrix of the brain, synovial fluid, umbilical cords and rooster combs from which it is isolated and purified, also can be produced by fermentation processes.

According to some embodiments, the pharmaceutical composition further comprises an adjuvant. Exemplary adjuvants include, but are not limited to, preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. Isotonic agents, for example, sugars, sodium chloride and the like, can also be included. Prolonged absorption of the injectable pharmaceutical form can be brought about by the use of agents delaying absorption, for example, aluminum monostearate and gelatin.

The formulations can be sterilized, for example, by terminal gamma irradiation, e beam irradiation, filtration through a bacterial-retaining filter or by incorporating sterilizing agents in the form of sterile solid compositions that may be dissolved or dispersed in sterile water or other sterile injectable medium just prior to use. Injectable preparations, for example, sterile injectable aqueous or oleaginous suspensions may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol, dichloromethane, ethyl acetate, acetonitrile, etc. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose any bland fixed oil may be employed including synthetic mono- or diglycerides. In addition, fatty acids such as oleic acid are used in the preparation of injectables.

Formulations for parenteral (including but not limited to, intraocular, intraorbital, subconjunctival, subcutaneous, intradermal, intramuscular, intravenous, intraarterial, intracisternal, intrathecal, intraventricular and intraarticular) administration include aqueous and non-aqueous sterile injection solutions that can contain anti-oxidants, buffers, bacteriostats and solutes, which render the formulation isotonic with the blood of the intended recipient; and aqueous and non-aqueous sterile suspensions, which can include suspending agents and thickening agents.

According to another embodiment, the pharmaceutical composition is formulated by conjugating the therapeutic agent to a polymer that enhances aqueous solubility. Examples of suitable polymers include but are not limited to polyethylene glycol, poly-(d-glutamic acid), poly-(1-glutamic acid), poly-(1-glutamic acid), poly-(d-aspartic acid), poly-(1-aspartic acid), poly-(1-aspartic acid) and copolymers thereof. Polyglutamic acids having molecular weights between about 5,000 to about 100,000, with molecular weights between about 20,000 and about 80,000 may be used and with molecular weights between about 30,000 and about 60,000 may also be used. The polymer is conjugated via an ester linkage to one or more hydroxyls of the therapeutic agent using a protocol as essentially described by U.S. Pat. No. 5,977,163 which is incorporated herein by reference.

Exemplary buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

### Delivery Systems

According to another aspect, the described invention provides a semisolid (meaning having a somewhat firm consistency) particulate delivery system for reducing visual loss and for treating one or more adverse consequence of an eye disease, including abnormal intraocular pressure, retinal vascular disease and retinal ganglion cell death, in order to reduce visual loss in a mammal in need thereof. According to some embodiments, the semisolid multiparticulate delivery system, when administered, can prevent or reduce the incidence or severity of visual loss and/or the adverse consequence (e.g., abnormal intraocular pressure, retinal vascular disease and retinal ganglion cell death) in order to reduce visual loss.

According to some embodiments, the pharmaceutical composition is administered by parenteral injection or surgical implantation.

According to some embodiments, the semisolid particulate delivery system comprises a cannula or catheter through which the pharmaceutical composition is delivered, wherein the catheter is inserted into a site of delivery in the mammal. According to one embodiment, the site of delivery is in close proximity to a site affected by the one or more adverse consequence of an eye disease, including abnormal intraocular pressure, retinal vascular disease and retinal ganglion cell death, in order to reduce visual loss. . According to another embodiment, the site of delivery is in close proximity to a blood vessel contributing to the one or more adverse consequence of an eye disease, including abnormal intraocular pressure, retinal vascular disease and retinal ganglion cell death in order to reduce visual loss.

According to some embodiments, the site of delivery is within 10 mm, less than 10 mm, less than 9.9 mm, less than 9.8 mm, less than 9.7 mm, less than 9.6 mm, less than 9.5 mm, less than 9.4 mm, less than 9.3 mm, less than 9.2 mm, less than 9.1 mm, less than 9.0 mm, less than 8.9 mm, less than 8.8 mm, less than 8.7 mm, less than 8.6 mm, less than 8.5 mm, less than 8.4 mm, less than 8.3 mm, less than 8.2 mm, less than 8.1 mm, less than 8.0 mm, less than 7.9 mm, less than 7.8 mm, less than 7.7 mm, less than 7.6 mm, less than 7.5 mm, less than 7.4 mm, less than 7.3 mm, less than 7.2 mm, less than 7.1 mm, less than 7.0 mm, less than 6.9 mm, less than 6.8 mm, less than 6.7 mm, less than 6.6 mm, less than 6.5 mm, less than 6.4 mm, less than 6.3 mm, less than 6.2 mm, less than 6.1 mm, less than 6.0 mm, less than 5.9 mm, less than 5.8 mm, less than 5.7 mm, less than 5.6 mm, less than 5.5 mm, less than 5.4 mm, less than 5.3 mm, less than 5.2 mm, less than 5.1 mm, less than 5.0 mm, less than 4.9 mm, less than 4.8 mm, less than 4.7 mm, less than 4.6 mm, less than 4.5 mm, less than 4.4 mm, less than 4.3 mm, less than 4.2 mm, less than 4.1 mm, less than 4.0 mm, less than 3.9 mm, less than 3.8 mm, less than 3.7 mm, less than 3.6 mm, less than 3.5 mm, less than 3.4 mm, less than 3.3 mm, less than 3.2 mm, less than 3.1 mm, less than 3.0 mm, less than 2.9 mm, less than 2.8 mm, less than 2.7 mm, less than 2.6 mm, less than 2.5 mm, less than 2.4 mm, less than 2.3 mm, less than 2.2 mm, less than 2.1 mm, less than 2.0 mm, less than 1.9 mm, less than 1.8 mm, less than 1.7 mm, less than 1.6 mm, less than 1.5 mm, less than 1.4 mm, less than 1.3 mm, less than 1.2 mm, less than 1.1 mm, less than 1.0 mm, less than 0.9 mm, less than 0.8 mm, less than 0.7 mm, less than 0.6 mm, less than 0.5 mm, less than 0.4 mm, less than 0.3 mm, less than 0.2 mm, less than 0.1 mm, less than 0.09 mm, less than 0.08 mm, less than 0.07 mm, less than 0.06 mm, less than 0.05 mm, less than 0.04 mm, less than 0.03 mm, less than 0.02 mm, less than 0.01 mm, less than 0.009 mm, less than 0.008 mm, less than 0.007 mm, less than 0.006 mm, less than 0.005 mm, less than 0.004 mm, less than 0.003 mm, less than 0.002 mm, less than 0.001 mm of a blood vessel contributing to the one or more adverse consequence of an eye disease, including abnormal intraocular pressure, retinal vascular disease and retinal ganglion cell death in order to reduce visual loss.

According to some embodiments, the pharmaceutical composition comprising the therapeutic agent(s) can be delivered to effectuate a localized release of a therapeutically effective amount of the therapeutic agent(s), thereby treating or reducing the incidence or severity of the one or more adverse consequence of an eye disease, including abnormal intraocular pressure, retinal vascular disease and retinal ganglion cell death in order to reduce visual loss and improving prognosis. Because the therapeutic agent(s) is/are delivered locally to the site affected by the one or more adverse consequence of an eye disease, including abnormal intraocular pressure, retinal vascular disease and retinal ganglion cell death or to a blood vessel contributing to the one or more adverse consequence of an eye disease, including abnormal intraocular pressure, retinal vascular disease and retinal ganglion cell death, the dosage required to treat or reduce the severity of the one or more adverse consequence of an eye disease, including abnormal intraocular pressure, retinal vascular disease and retinal ganglion cell death is lower, thereby circumventing unwanted side effects associated with systemic delivery of higher doses, such as hypotension.

According to one embodiment, the pharmaceutical composition comprising the therapeutic agent(s) can be delivered by inserting a catheter and injecting the pharmaceutical composition through the catheter such that the pharmaceutical composition emanates from the end of the catheter locally.

According to another embodiment, the pharmaceutical composition is given as a single bolus injection. According to another embodiment, the injection is repeated after a pre-determined time period. According to some such embodiments, the pre-determined time period could range from 1 minute or more to 10 days or more. For example, a repeat injection can be given if monitoring of the patient showed that the patient still had evidence of retinal vascular disease.

More generally, the semisolid particulate delivery system of the described invention provides a number of advantages over systemic administration either orally or by infusion. As an example, nimodipine now must be administered as a continuous intravenous infusion or orally as pills given every 2 to 4 hours. The concentration of the therapeutic agent locally where it exerts its effect is higher and the plasma concentration is lower than when the therapeutic agent is administered orally or intravenously. This results in a localized pharmacological effect in arteries that perfuse the retina either directly or indirectly, with less effect in the body. Side effects in the body, such as hypotension, are less likely to occur. The total dose of therapeutic agents administered thus is much lower than that administered systemically so the risk of other and unknown side effects is lower.

According to some embodiments, the pharmaceutical composition comprising the therapeutic agent is contained in a controlled release delivery system. Controlled release systems deliver a drug at a predetermined rate for a definite time period. (Reviewed in Langer, R., “New methods of drug delivery,” Science, 249: 1527-1533 (1990); and Langer, R., “Drug delivery and targeting,” Nature, 392 (Supp.): 5-10 (1998)). Generally, release rates are determined by the design of the system, and are nearly independent of environmental conditions, such as pH. These systems also can deliver drugs for long time periods (days or years). Controlled release systems provide advantages over conventional drug therapies. For example, after ingestion or injection of standard dosage forms, the blood level of the drug rises, peaks and then declines. Since each drug has a therapeutic range above which it is toxic and below which it is ineffective, oscillating drug levels may cause alternating periods of ineffectiveness and toxicity. A controlled release preparation maintains the drug in the desired therapeutic range by a single administration. Other potential advantages of controlled release systems include: (i) localized delivery of the drug to a particular body compartment, thereby lowering the systemic drug level; (ii) preservation of medications that are rapidly destroyed by the body; (iii) reduced need for follow-up care; (iv) increased comfort; and (v) improved compliance. (Langer, R., “New methods of drug delivery,” Science, 249: at 1528).

According to some embodiments, control is afforded by placing the drug in a polymeric material or pump. Polymeric materials generally release drugs by the following mechanisms: (i) diffusion; (ii) chemical reaction, or (iii) solvent activation. The most common release mechanism is diffusion. In this approach, the drug is physically entrapped inside a solid polymer that can then be injected or implanted in the body. The drug then migrates from its initial position in the polymeric system to the polymer's outer surface and then to the body. There are two types of diffusion-controlled systems: reservoirs, in which a drug core is surrounded by a polymer film, which produce near-constant release rates, and matrices, where the drug is uniformly distributed through the polymer system. Drugs also can be released by chemical mechanisms, such as degradation of the polymer, or cleavage of the drug from a polymer backbone. Exposure to a solvent also can activate drug release; for example, the drug may be locked into place by polymer chains, and, upon exposure to environmental fluid, the outer polymer regions begin to swell, allowing the drug to move outward, or water may permeate a drug-polymer system as a result of osmotic pressure, causing pores to form and bringing about drug release. Such solvent-controlled systems have release rates independent of pH. Some polymer systems can be externally activated to release more drug when needed. Release rates from polymer systems can be controlled by the nature of the polymeric material (for example, crystallinity or pore structure for diffusion-controlled systems; the lability of the bonds or the hydrophobicity of the monomers for chemically controlled systems) and the design of the system (for example, thickness and shape). (Langer, R., “New methods of drug delivery,” Science, 249: at 1529).

Polyesters such as lactic acid-glycolic acid copolymers display bulk (homogeneous) erosion, resulting in significant degradation in the matrix interior. To maximize control over release, it is often desirable for a system to degrade only from its surface. For surface-eroding systems, the drug release rate is proportional to the polymer erosion rate, which eliminates the possibility of dose dumping, improving safety; release rates can be controlled by changes in system thickness and total drug content, facilitating device design. Achieving surface erosion requires that the degradation rate on the polymer matrix surface be much faster than the rate of water penetration into the matrix bulk. Theoretically, the polymer should be hydrophobic but should have water-labile linkages connecting monomers. For example, it was proposed that, because of the lability of anhydride linkages, polyanhydrides would be a promising class of polymers. By varying the monomer ratios in polyanhydride copolymers, surface-eroding polymers lasting from 1 week to several years were designed, synthesized and used to deliver nitrosoureas locally to the brain. ((Langer, R., “New methods of drug delivery,” Science, 249: at 1531 citing.Rosen et al, Biomaterials 4, 131 (1983); Leong et al, J. Biomed. Mater. Res. 19, 941 (1985); Domb et al, Macromolecules 22, 3200 (1989); Leong et al, J. Biomed. Mater. Res. 20, 51 (1986), Brem et al, Selective Cancer Ther. 5, 55 (1989); Tamargo et al, J. Biomed. Mater. Res. 23, 253 (1989)).

Several different surface-eroding polyorthoester systems have been synthesized. Additives are placed inside the polymer matrix, which causes the surface to degrade at a different rate than the rest of the matrix. Such a degradation pattern can occur because these polymers erode at very different rates, depending on pH, and the additives maintain the matrix bulk at a pH different from that of the surface. By varying the type and amount of additive, release rates can be controlled. ((Langer, R., “New methods of drug delivery,” Science, 249: at 1531 citing Heller, etal, in Biodegradable Polymers as Drug Delivery Systems, M. Chasin and R. Langer, Eds (Dekker, New York, 1990), pp. 121-161)).

Polymeric materials used in controlled release drug delivery systems described for delivery to the CNS include poly (a-hydroxyacids), acrylic, polyanhydrides and other polymers, such as polycaprolactone, ethylcellulose, polystyrene, etc. A wide range of delivery systems suitable for delivery to the brain and spinal cord have been developed. These include: macroscopic implants, microcapsules, gels and nanogels, microparticles/microspheres, nanoparticles, and composite hydrogel systems. The different types of systems exhibit differences in pharmokinetic and pharmacodynamic profiles of drugs by affecting different physical and chemical processes involved in drug release, such as water penetration, drug dissolution, and degradation of matrix and drug diffusion. (Reviewed in Siepmann, J. et al., “Local controlled drug delivery to the brain: mathematical modeling of the underlying mass transport mechanisms,” International Journal of Pharmaceutics, 314: 101-119 (2006).

According to some embodiments, in order to prolong the effect of a drug, it often is desirable to slow the absorption of the drug. This can be accomplished by the use of a liquid suspension of crystalline or amorphous material with poor water solubility. The rate of absorption of the drug then depends upon its rate of dissolution which, in turn, may depend upon crystal size and crystalline form. For example, according to some embodiments, a SABER™ Delivery System comprising a high-viscosity base component, is used to provide controlled release of the therapeutic agent. (See U.S. Pat. No. 8,168,217, U.S. Pat. No. 5,747,058 and U.S. Pat. No. 5,968,542, incorporated herein by reference). When the high viscosity SAIB is formulated with drug, biocompatible excipients and other additives, the resulting formulation is liquid enough to inject easily with standard syringes and needles. After injection of a SABER™ formulation, the excipients diffuse away, leaving a viscous depot.

SABER™ formulations comprise a drug and a high viscosity liquid carrier material (HVLCM), meaning nonpolymeric, nonwater soluble liquids with a viscosity of at least 5,000 cP at 37° C. that do not crystallize neat under ambient or physiological conditions. HVLCMs may be carbohydrate-based, and may include one or more cyclic carbohydrates chemically combined with one or more carboxylic acids, such as sucrose acetate isobutyrate (SAIB). HVLCMs also include nonpolymeric esters or mixed esters of one or more carboxylic acids, having a viscosity of at least 5,000 cP at 37° C., that do not crystallize neat under ambient or physiological conditions, wherein when the ester contains an alcohol moiety (e.g., glycerol). The ester may, for example comprise from about 2 to about 20 hydroxy acid moieties.

Additional components can include, without limitation, a rheology modifier, and/or a network former. A rheology modifier is a substance that possesses both a hydrophobic and a hydrophilic moiety used to modify viscosity and flow of a liquid formulation, for example, caprylic/capric triglyceride (Migliol 810), isopropyl myristate (IM or IPM), ethyl oleate, triethyl citrate, dimethyl phthalate, and benzyl benzoate. A network former is a compound that forms a network structure when introduced into a liquid medium. Exemplary network formers include cellulose acetate butyrate, carbohydrate polymers, organic acids of carbohydrate polymers and other polymers, hydrogels, as well as particles such as silicon dioxide, ion exchange resins, and/or fiberglass, that are capable of associating, aligning or congealing to form three dimensional networks in an aqueous environment.

According to some embodiments, one half of the therapeutic agent is released from the controlled release system at the site of delivery within 1 day to at least 365 days in vivo. According to one embodiment, one-half of the therapeutic agent is released from the controlled release system at the site of delivery within 1 day. According to another embodiment, one-half of the therapeutic agent is released from the controlled release system at the site of delivery within 2 days. According to another embodiment, one-half of the therapeutic agent is released from the controlled release system at the site of delivery within 3 days. According to another embodiment, one-half of the therapeutic agent is released from the controlled release system at the site of delivery within 4 days. According to another embodiment, one-half of the therapeutic agent is released from the controlled release system at the site of delivery within 5 days. According to another embodiment, one-half of the therapeutic agent is released from the controlled release system at the site of delivery within 6 days. According to another embodiment, one-half of the therapeutic agent is released from the controlled release system at the site of delivery within 7 days. According to another embodiment, one-half of the therapeutic agent is released from the controlled release system at the site of delivery within 8 days. According to another embodiment, one-half of the therapeutic agent is released from the controlled release system at the site of delivery within 9 days. According to another embodiment, one-half of the therapeutic agent is released from the controlled release system at the site of delivery within 10 days. According to another embodiment, one-half of the therapeutic agent is released from the controlled release system at the site of delivery within 15 days. According to another embodiment, one-half of the therapeutic agent is released from the controlled release system at the site of delivery within 20 days. According to another embodiment, one-half of the therapeutic agent is released from the controlled release system at the site of delivery within 30 days. According to another embodiment, one-half of the therapeutic agent is released from the controlled release system at the site of delivery within 40 days. According to another embodiment, one-half of the therapeutic agent is released from the controlled release system at the site of delivery within a half-life of 50 days. According to another embodiment, one-half of the therapeutic agent is released from the controlled release system at the site of delivery within 60 days. According to another embodiment, one-half of the therapeutic agent is released from the controlled release system at the site of delivery within 70 days.

According to another embodiment, one-half of the therapeutic agent is released from the pharmaceutical composition at the site of delivery within 80 days. According to another embodiment, one-half of the therapeutic agent is released from the pharmaceutical composition at the site of delivery within 90 days. According to another embodiment, one-half of the therapeutic agent is released from the pharmaceutical composition at the site of delivery within 100 days. According to another embodiment, one-half of the therapeutic agent is released from the pharmaceutical composition at the site of delivery within 110 days. According to another embodiment, one-half of the therapeutic agent is released from the pharmaceutical composition at the site of delivery within 120 days. According to another embodiment, one-half of the therapeutic agent is released from the pharmaceutical composition at the site of delivery within 140 days. According to another embodiment, one-half of the therapeutic agent is released from the pharmaceutical composition at the site of delivery within 150 days. According to another embodiment, one-half of the therapeutic agent is released from the pharmaceutical composition at the site of delivery within 160 days. According to another embodiment, one-half of the therapeutic agent is released from the pharmaceutical composition at the site of delivery within 170 days. According to another embodiment, one-half of the therapeutic agent is released from the pharmaceutical composition at the site of delivery within 180 days. According to another embodiment, one-half of the therapeutic agent is released from the pharmaceutical composition at the site of delivery within 190 days. According to another embodiment, one-half of the therapeutic agent is released from the pharmaceutical composition at the site of delivery within 200 days. According to another embodiment, one-half of the therapeutic agent is released from the pharmaceutical composition at the site of delivery within 210 days. According to another embodiment, one-half of the therapeutic agent is released from the pharmaceutical composition at the site of delivery within 220 days. According to another embodiment, one-half of the therapeutic agent is released from the pharmaceutical composition at the site of delivery within 230 days. According to another embodiment, one-half of the therapeutic agent is released from the pharmaceutical composition at the site of delivery within 240 days. According to another embodiment, one-half of the therapeutic agent is released from the pharmaceutical composition at the site of delivery within 250 days. According to another embodiment, one-half of the therapeutic agent is released from the pharmaceutical composition at the site of delivery within 260 days. According to another embodiment, one-half of the therapeutic agent is released from the pharmaceutical composition at the site of delivery within 270 days. According to another embodiment, one-half of the therapeutic agent is released from the pharmaceutical composition at the site of delivery within 280 days. According to another embodiment, one-half of the therapeutic agent is released from the pharmaceutical composition at the site of delivery within 290 days. According to another embodiment, one-half of the therapeutic agent is released from the pharmaceutical composition at the site of delivery within 300 days. According to another embodiment, one-half of the therapeutic agent is released from the pharmaceutical composition at the site of delivery within 310 days. According to another embodiment, one-half of the therapeutic agent is released from the pharmaceutical composition at the site of delivery within 310 days. According to another embodiment, one-half of the therapeutic agent is released from the pharmaceutical composition at the site of delivery within 320 days. According to another embodiment, one-half of the therapeutic agent is released from the pharmaceutical composition at the site of delivery within 330 days. According to another embodiment, one-half of the therapeutic agent is released from the pharmaceutical composition at the site of delivery within 340 days. According to another embodiment, one-half of the therapeutic agent is released from the pharmaceutical composition at the site of delivery within 350 days. According to another embodiment, one-half of the therapeutic agent is released from the pharmaceutical composition at the site of delivery within 360 days. According to another embodiment, one-half of the therapeutic agent is released from the pharmaceutical composition at the site of delivery within at least 365 days.

According to some embodiments, the controlled release system comprises a long term sustained release implant that can be particularly suitable for treatment of chronic conditions. The term “long-term” release, as used herein, means that the implant is constructed and arranged to deliver therapeutic levels of the active ingredient for at least 7 days, and preferably about 30 days to about 60 days. Long-term sustained release implants are well-known to those of ordinary skill in the art and include some of the release systems described above.

According to another embodiment, the release of the therapeutic agent at the site of delivery can produce a predominantly localized pharmacologic effect over a desired amount of time. According to one embodiment, the release of the therapeutic agent at the site of delivery produces a predominantly localized pharmacologic effect for 1 day. According to one embodiment, the release of the therapeutic agent at the site of delivery produces a predominantly localized pharmacologic effect for 2 days. According to one embodiment, the release of the therapeutic agent at the site of delivery produces a predominantly localized pharmacologic effect for 3 days. According to one embodiment, the release of the therapeutic agent at the site of delivery produces a predominantly localized pharmacologic effect for 4 days. According to one embodiment, the release of the therapeutic agent at the site of delivery produces a predominantly localized pharmacologic effect for 5 days. According to one embodiment, the release of the therapeutic agent at the site of delivery produces a predominantly localized pharmacologic effect for 6 days. According to one embodiment, the release of the therapeutic agent at the site of delivery produces a predominantly localized pharmacologic effect for 7 days. According to one embodiment, the release of the therapeutic agent at the site of delivery produces a predominantly localized pharmacologic effect for 8 days. According to one embodiment, the release of the therapeutic agent at the site of delivery produces a predominantly localized pharmacologic effect for 9 days. According to one embodiment, the release of the therapeutic agent at the site of delivery produces a predominantly localized pharmacologic effect for 10 days. According to one embodiment, the release of the therapeutic agent at the site of delivery produces a predominantly localized pharmacologic effect for 15 days. According to one embodiment, the release of the therapeutic agent at the site of delivery produces a predominantly localized pharmacologic effect for 20 days. According to one embodiment, the release of the therapeutic agent at the site of delivery produces a predominantly localized pharmacologic effect for 25 days. According to one embodiment, the release of the therapeutic agent at the site of delivery produces a predominantly localized pharmacologic effect for 30 days. According to one embodiment, the release of the therapeutic agent at the site of delivery produces a predominantly localized pharmacologic effect for 35 days. According to one embodiment, the release of the therapeutic agent at the site of delivery produces a predominantly localized pharmacologic effect for 40 days. According to one embodiment, the release of the therapeutic agent at the site of delivery produces a predominantly localized pharmacologic effect for 45 days. According to one embodiment, the release of the therapeutic agent at the site of delivery produces a predominantly localized pharmacologic effect for 50 days. According to one embodiment, the release of the therapeutic agent at the site of delivery produces a predominantly localized pharmacologic effect for 60 days. According to one embodiment, the release of the therapeutic agent at the site of delivery produces a predominantly localized pharmacologic effect for 75 days. According to one embodiment, the release of the therapeutic agent at the site of delivery produces a predominantly localized pharmacologic effect for 90 days. According to one embodiment, the release of the therapeutic agent at the site of delivery produces a predominantly localized pharmacologic effect for 120 days. According to one embodiment, the release of the therapeutic agent at the site of delivery produces a predominantly localized pharmacologic effect for 150 days. According to one embodiment, the release of the therapeutic agent at the site of delivery produces a predominantly localized pharmacologic effect for 180 days.

According to another embodiment, the release of the therapeutic agent at the site of delivery produces a diffuse pharmacologic effect throughout the eye over a desired amount of time. According to another embodiment, the release of the therapeutic agent can produce a diffuse pharmacologic effect throughout the eye for at least 1 day. According to another embodiment, the release of the therapeutic agent can produce a diffuse pharmacologic effect throughout the eye for at least 2 days. According to another embodiment, the release of the therapeutic agent can produce a diffuse pharmacologic effect throughout the eye for at least 3 days. According to another embodiment, the release of the therapeutic agent can produce a diffuse pharmacologic effect throughout the eye for at least 4 days. According to another embodiment, the release of the therapeutic agent can produce a diffuse pharmacologic effect throughout the eye for at least 5 days. According to another embodiment, the release of the therapeutic agent can produce a diffuse pharmacologic effect throughout the eye for at least 6 days. According to another embodiment, the release of the therapeutic agent can produce a diffuse pharmacologic effect throughout the eye for at least 7 days. According to another embodiment, the release of the therapeutic agent can produce a diffuse pharmacologic effect throughout the eye for at least 8 days. According to another embodiment, the release of the therapeutic agent can produce a diffuse pharmacologic effect throughout the eye for at least 9 days. According to another embodiment, the release of the therapeutic agent can produce a diffuse pharmacologic effect throughout the eye for at least 10 days. According to another embodiment, the release of the therapeutic agent can produce a diffuse pharmacologic effect throughout the eye for at least 15 days. According to another embodiment, the release of the therapeutic agent can produce a diffuse pharmacologic effect throughout the eye for at least 20 days. According to another embodiment, the release of the therapeutic agent can produce a diffuse pharmacologic effect throughout the eye for at least 25 days. According to another embodiment, the release of the therapeutic agent can produce a diffuse pharmacologic effect for throughout the eye for at least 30 days. According to another embodiment, the release of the therapeutic agent can produce a diffuse pharmacologic effect throughout the eye for at least 35 days. According to another embodiment, the release of the therapeutic agent can produce a diffuse pharmacologic effect throughout the eye for at least 40 days. According to another embodiment, the release of the therapeutic agent can produce a diffuse pharmacologic effect throughout the eye for at least 45 days. According to another embodiment, the release of the therapeutic agent can produce a diffuse pharmacologic effect throughout the eye for at least 50 days. According to another embodiment, the release of the therapeutic agent can produce a diffuse pharmacologic effect throughout the eye for at least 55 days. According to another embodiment, the release of the therapeutic agent can produce a diffuse pharmacologic effect throughout the eye for at least 60 days. According to another embodiment, the release of the therapeutic agent can produce a diffuse pharmacologic effect throughout the eye for at least 75 days. According to another embodiment, the release of the therapeutic agent can produce a diffuse pharmacologic effect throughout the eye for at least 90 days. According to another embodiment, the release of the therapeutic agent can produce a diffuse pharmacologic effect throughout the eye for at least 120 days. According to another embodiment, the release of the therapeutic agent can produce a diffuse pharmacologic effect throughout the eye for at least 150 days. According to another embodiment, the release of the therapeutic agent can produce a diffuse pharmacologic effect throughout the eye for at least 180 days.

According to one embodiment, the pharmaceutical composition is effective to increase ocular blood flow as compared to a control. According to another embodiment, the pharmaceutical composition is effective to increase ocular blood flow, optic nerve blood flow, optic nerve head blood flow, retrobulbar blood flow, retinal blood flow, choroidal blood flow and ocular perfusion.

### Means of Administration

According to some embodiments, the administering of the pharmaceutical composition can be, for example, into the eye, into the orbit, or into the subconjunctival space. According to some embodiments, administering into the eye comprises administering to the vitreous humor, the aqueous humor, or both.

According to one embodiment, the means for administering includes, but is not limited to, injection, a catheter, a punctual plug, a polymerized collagen gel, a contact lens, and the like.

Non-limiting examples of contact lenses include a soft contact lens, a gas permeable lens and a hybrid contact lens.

Soft contacts are made of hydrophilic plastic polymers called hydrogels. These materials can absorb water and become soft and pliable without losing their optical qualities.

Soft contacts—including new highly oxygen-permeable varieties called silicone hydrogel lenses—can be made with either a lathe cutting process or an injection molding process.

In the lathe cutting process, non-hydrated disks (or “buttons”) of soft contact lens material are individually mounted on spinning shafts and are shaped with computer-controlled precision cutting tools. After the front and back surfaces are shaped with the cutting tool, the lens is then removed from the lathe and hydrated to soften it. The finished lenses then undergo quality assurance testing. Though the lathe cutting process has more steps and is more time-consuming than an injection molding process, over the years the process has become more automated. With computers and industrial robotics, it now takes only a few minutes to create a lathe-cut soft contact lens.

In the injection molding process, the soft contact lens material is heated to a molten state and is then injected into computer-designed molds under pressure. The lenses are then quickly cooled and removed from the molds. The edges of the lenses are polished smooth, and the lenses are hydrated to soften them prior to undergoing quality assurance testing. Most disposable contact lenses are made with an injection molding process, as this method is faster and less expensive than lathe cutting processes.

Most rigid gas permeable lenses (RGP or GP lenses) are made of oxygen-permeable plastic polymers containing silicone and fluorine. GP lenses contain very little water and remain rigid on the eye. Gas permeable lenses are custom-made to specifications supplied by the prescribing doctor and hence are more costly than mass-produced soft lenses. A greater degree of customization is needed for GP contacts because they maintain their shape and do not conform to the eye like soft lenses. Minute differences in lens design can be the difference between a comfortable fit and contact lens failure with gas permeable lenses. GP contacts are made with a computerized precision lathe cutting process similar to that used for lathe-cut soft lenses. These lenses are shipped dry to the prescribing doctor. The doctor's office then soaks the lenses in a GP contact lens care solution prior to dispensation to the patient. This solution “conditions” the lens surfaces for greater wearing comfort.

Hybrid contact lenses have a central optical zone made of rigid gas permeable plastic, surrounded by a peripheral fitting zone made of a soft contact lens material. Hybrid lenses are made with a process very similar to lathe-cut soft contact lenses, with one very significant difference: the plastic disks cut with the lathe have a GP center, surrounded by non-hydrated soft contact lens material. The two materials are bonded together with proprietary technology to prevent separation of the materials after the lenses are cut and hydrated.

### Pharmaceutical Composition

### Voltage-Gated Calcium Channel Antagonist

Non-limiting examples of the voltage-gated calcium channel antagonist that can be formulated into the composition include, but are not limited to, L-type voltage-gated calcium channel antagonist, N-type voltage-gated calcium channel antagonist, P/Q-type voltage-gated calcium channel antagonist, or a combination thereof.

For example, L-type voltage-gated calcium channel antagonists include, but are not limited to: dihydropyridine L-type antagonists such as nisoldipine, nicardipine, nilvadipine, and nifedipine, AHF (such as 4aR,9aS)-(+)-4a-Amino-1,2,3,4,4a,9a-hexahydro-4aH-fluorene, HCl), isradipine (such as 4-(4-Benzofurazanyl)-1,-4-dihydro-2,6-dimethyl-3,5-pyridinedicarboxylic acid methyl 1-methhylethyl ester), calciseptine (such as isolated from (Dendroaspis polylepis ploylepis), H-Arg-Ile-Cys-Tyr-Ile-His-Lys-Ala-Ser-Leu-Pro-Arg-Ala-Thr-Lys-Thr-Cys-Val-Glu-Asn-Thr-Cys-Tyr-Lys-Met-Phe-Ile-Arg-Thr-Gln-Arg-Glu-Tyr-Ile-Ser-Glu-Arg-Gly-Cys-Gly-Cys-Pro-Thr-Ala-Met-Trp- Pro-Tyr-Gln-Thr-Glu-Cys-Cys-Lys-Gly-Asp-Arg-Cys-Asn-Lys-OH, Calcicludine (such as isolated from Dendroaspis angusticeps (eastern green mamba)),(H-Trp-Gln-Pro-Pro-Trp-Tyr-Cys-Lys-Glu-Pro-Val-Arg-Ile-Gly-Ser-Cys-Lys-Lys-Gln-Phe-Ser-Ser-Phe-Tyr-Phe-Lys-Trp-Thr-Ala-Lys-Lys-Cys-Leu-Pro-Phe-Leu-Phe-Ser-Gly-Cys-Gly-Gly-Asn-Ala-Asn-Arg-Phe-Gln- Thr-Ile-Gly-Glu-Cys-Arg-Lys-Lys-Cys-Leu-Gly-Lys-OH, Cilnidipine (such as also FRP-8653, a dihydropyridine-type inhibitor), Dilantizem (such as (2S,3S)-(+)-cis-3-Acetoxy-5-(2-dimethylaminoethyl)-2,3-dihydro-2-(4-methoxyphenyl)-1,5-benzothiazepin-4(5H)-one hydrochloride), diltiazem (such as benzothiazepin-4(5H)-one, 3-(acetyloxy)-5-[2-(dimethylamino)ethyl]-2,3-dihydro-2-(4-methoxyphenyl)-,(+)-cis-, monohydrochloride), Felodipine (such as 4-(2,3-Dichlorophenyl)-1,4-dihydro-2,6-dimethyl-3,5-pyridinecarboxylic acid ethyl methyl ester), FS-2 (such as an isolate from Dendroaspis polylepis polylepis venom), FTX-3.3 (such as an isolate from Agelenopsis aperta), Neomycin sulfate (such as C₂₃H₄₆N₆O₁₃·3H₂SO₄), Nicardipine (such as 1,4-Dihydro-2,6-dimethyl-4-(3-nitrophenypmethyl-2-[methyl(phenylmethypa-mino]-3,5-pyridinedicarboxylic acid ethyl ester hydrochloride, also YC-93, Nifedipine (such as 1,4-Dihydro-2,6-dimethyl-4-(2-nitrophenyl)-3,5-pyridinedicarboxylic acid dimethyl ester), Nimodipine (such as 4-Dihydro-2,6-dimethyl-4-(3-nitrophenyl)-3,5-pyridinedicarboxylic acid 2-methoxyethyl 1-methylethyl ester) or (Isopropyl 2-methoxyethyl 1,4-dihydro-2,6-dimethyl-4-(m-nitrophenyl)-3,5-pyridinedicarboxylate), Nitrendipine (such as 1,4-Dihydro-2,6-dimethyl-4-(3-nitrophenyl)-3,5-pyridinedicarboxylic acid ethyl methyl ester), S-Petasin (such as (3 S,4aR,5R,6R)-[2,3,4,4a,5,6,7,8-Octahydro-3-(2-propenyl)-4a,5-dimethyl-2-o-xo-6-naphthyl]Z-3′-methylthio-1′-propenoate), Phloretin (such as 2′,4′,6′-Trihydroxy-3-(4-hydroxyphenyl)propiophenone, also 3-(4-Hydroxyphenyl)-1-(2,4,6-trihydroxyphenyl)-1-propanone, also b-(4-Hydroxyphenyl)-2,4,6-trihydroxypropiophenone), Protopine (such as C₂₀H₁₉NO₅Cl), SKF-96365 (such as 1-[b-[3-(4-Methoxyphenyl)propoxy]-4-methoxyphenethyl]-1H-imidazole, HCl), Tetrandine (such as 6,6′,7,12-Tetramethoxy-2,2′-dimethylberbaman), (.+-.)-Methoxyverapamil or (+)-Verapamil (such as 54N-(3,4-Dimethoxyphenylethyl)methylamino]-2-(3,4-dimethoxyphenyl)-2-iso-propylvaleronitrile hydrochloride), and (R)-(+)-B ay K8644 (such as R-(+)-1,4-Dihydro-2,6-dimethyl-5-nitro-442-(trifluoromethyl)phenyl]-3-pyridinecarboxylic acid methyl ester). The foregoing examples may be specific to L-type voltage-gated calcium channels or may inhibit a broader range of voltage-gated calcium channels, e.g. N, P/Q, R, and T-type.

According to some embodiments, the voltage-gated calcium channel antagonist is a dihydropyridine calcium channel antagonist. According to one embodiment, the dihydropyridine calcium channel antagonist is nimodipine. According to one embodiment, the nimodipine has a half-life of 7-10 days when formulated as described herein, and appropriate lipid solubility.

According to some embodiments, the therapeutic agent is an isolated molecule. The term “isolated molecule” as used herein refers to a molecule that is substantially pure and is free of other substances with which it is ordinarily found in nature or in vivo systems to an extent practical and appropriate for its intended use.

According to some embodiments, the therapeutic agent is admixed with a pharmaceutically-acceptable carrier in a pharmaceutical preparation. According to some such embodiments, the therapeutic agent comprises only a small percentage by weight of the preparation. According to some embodiments, the therapeutic agent is substantially pure.

### Endothelin Receptor Antagonist

According to some embodiments, ETA-receptor antagonists may include, but are not limited to, A-127722 (non-peptide), ABT-627 (non-peptide), BMS 182874 (non-peptide), BQ-123 (peptide), BQ-153 (peptide), BQ-162 (peptide), BQ-485 (peptide), BQ-518 (peptide), BQ-610 (peptide), EMD-122946 (non-peptide), FR 139317 (peptide), IPI-725 (peptide), L-744453 (non-peptide), LU 127043 (non-peptide), LU 135252 (non-peptide), PABSA (non-peptide), PD 147953 (peptide), PD 151242 (peptide), PD 155080 (non-peptide), PD 156707 (non-peptide), RO 611790 (non-peptide), SB-247083 (non-peptide), clazosentan (non-peptide), atrasentan (non-peptide), sitaxsentan sodium (non-peptide), TA-0201 (non-peptide), TBC 11251 (non-peptide), TTA-386 (peptide), WS-7338B (peptide), ZD-1611 (non-peptide), and aspirin (non-peptide). ETA/B-receptor antagonists may include, but are not limited to, A-182086 (non-peptide), CGS 27830 (non-peptide), CP 170687 (non-peptide), J-104132 (non-peptide), L-751281 (non-peptide), L-754142 (non-peptide), LU 224332 (non-peptide), LU 302872 (non-peptide), PD 142893 (peptide), PD 145065 (peptide), PD 160672 (non-peptide), RO-470203 (bosentan, non-peptide), RO 462005 (non-peptide), RO 470203 (non-peptide), SB 209670 (non-peptide), SB 217242 (non-peptide), and TAK-044 (peptide). ETB-ireceptor antagonists may include, but are not limited to, A-192621 (non-peptide), A-308165 (non-peptide), BQ-788 (peptide), BQ-017 (peptide), IRL 1038 (peptide), IRL 2500 (peptide), PD-161721 (non-peptide), RES 701-1 (peptide), and RO 468443 (peptide).

According to some embodiments, the flowable particulate composition further comprises a therapeutic amount of one or more additional therapeutic agent(s). According to some embodiments, the additional therapeutic agent is a prostaglandin analog. According to some such embodiments, the prostaglandin analog is latanoprost. According to some embodiments, the additional therapeutic agent is one or more Rho kinase inhibitor. According to some such embodiments, exemplary Rho kinase inhibitors include, without limitation, Y-27632 2HCl (R&D Systems Inc., Minneapolis, Minn.), Triazovivin® (StemRD, Burlingame, Calif.), Slx-2119 (MedChem Express, Namiki Shoji Cop., LTD), WF-536 [(+)-®-4-(1-aminoethyl)-N-(4-pyridyl) benzamide monohydrochloride] (Mitsubishi Pharma Corporation, Osaka, Japan), RK1-1447 (University of South Florida, Tampa, Fla., and Moffitt Cancer Center, Tampa, Fla.; Roberta Pireddu et al., “Pyridylthiazole-based ureas as inhibitors of Rho associated protein kinases (ROCK1 and 2).” (2012) Medchemcomm. 3(6):699-709), Fasudil® (Asahi-KASEI Corp., Osaka, Japan), Fasudil® hydrochloride (R&D Systems Inc., Minneapolis, Minn.), GSK429286A (R&D Systems Inc., Minneapolis, Minn.), Rockout® (EMD Millipore, Philadelphia, Pa.), SR 3677 dihydrochloride (R&D Systems Inc., Minneapolis, Minn.); SB 772077B (R&D Systems Inc., Minneapolis, Minn.), AS 1892802 (R&D Systems Inc., Minneapolis, Minn.), H 1152 dihydrochloride (R&D Systems Inc., Minneapolis, Minn.), GSK 269962 (R&D Systems Inc., Minneapolis, Minn.), HA 1100 hydrochloride (R&D Systems Inc., Minneapolis, Minn.), Glycyl-H-1152 dihydrochloride (R&D Systems Inc., Minneapolis, Minn.), AR-12286 (Aerie Pharmaceuticals), AR-13324 (Rhopressa, Aerie Pharmaceuticals), AMA-0076 (Amakem Therapeutics), and K-115 (Kumatomo University, Japan). According to some other embodiments, the additional therapeutic agent includes a combination of a Rho kinase inhibitor and a prostaglandin analog.

### Pharmaceutically Acceptable Carrier

According to some embodiments, the pharmaceutical composition comprises a pharmaceutically acceptable carrier.

According to one embodiment, the pharmaceutically acceptable carrier is a solid carrier or excipient. According to another embodiment, the pharmaceutically acceptable carrier is a gel-phase carrier or excipient. Examples of carriers or excipients include, but are not limited to, calcium carbonate, calcium phosphate, various monomeric and polymeric sugars (including without limitation hyaluronic acid), starches, cellulose derivatives, gelatin, and polymers. An exemplary carrier can also include saline vehicle, e.g. hydroxyl propyl methyl cellulose (HPMC) in phosphate buffered saline (PBS).

According to some embodiments, the pharmaceutically acceptable carrier imparts stickiness. According to one embodiment, the pharmaceutically acceptable carrier comprises hyaluronic acid. According to some embodiments, the pharmaceutically acceptable carrier comprises 0% to 5% hyaluronic acid. According to one embodiment, the pharmaceutically acceptable carrier comprises less than 0.05% hyaluronic acid. According to another embodiment, the pharmaceutically acceptable carrier comprises less than 0.1% hyaluronic acid. According to another embodiment, the pharmaceutically acceptable carrier comprises less than 0.2% hyaluronic acid. According to another embodiment, the pharmaceutically acceptable carrier comprises less than 0.3% hyaluronic acid. According to another embodiment, the pharmaceutically acceptable carrier comprises less than 0.4% hyaluronic acid. According to another embodiment, the pharmaceutically acceptable carrier comprises less than 0.5% hyaluronic acid. According to another embodiment, the pharmaceutically acceptable carrier comprises less than 0.6% hyaluronic acid. According to another embodiment, the pharmaceutically acceptable carrier comprises less than 0.7% hyaluronic acid. According to another embodiment, the pharmaceutically acceptable carrier comprises less than 0.8% hyaluronic acid. According to another embodiment, the pharmaceutically acceptable carrier comprises less than 0.9% hyaluronic acid. According to another embodiment, the pharmaceutically acceptable carrier comprises less than 1.0% hyaluronic acid. According to another embodiment, the pharmaceutically acceptable carrier comprises less than 1.1% hyaluronic acid. According to another embodiment, the pharmaceutically acceptable carrier comprises less than 1.2% hyaluronic acid. According to another embodiment, the pharmaceutically acceptable carrier comprises less than 1.3% hyaluronic acid. According to another embodiment, the pharmaceutically acceptable carrier comprises less than 1.4% hyaluronic acid. According to another embodiment, the pharmaceutically acceptable carrier comprises less than 1.5% hyaluronic acid. According to another embodiment, the pharmaceutically acceptable carrier comprises less than 1.6% hyaluronic acid. According to another embodiment, the pharmaceutically acceptable carrier comprises less than 1.7% hyaluronic acid. According to another embodiment, the pharmaceutically acceptable carrier comprises less than 1.8% hyaluronic acid. According to another embodiment, the pharmaceutically acceptable carrier comprises less than 1.9% hyaluronic acid. According to another embodiment, the pharmaceutically acceptable carrier comprises less than 2.0% hyaluronic acid. According to another embodiment, the pharmaceutically acceptable carrier comprises less than 2.1% hyaluronic acid. According to another embodiment, the pharmaceutically acceptable carrier comprises less than 2.2% hyaluronic acid. According to another embodiment, the pharmaceutically acceptable carrier comprises less than 2.3% hyaluronic acid. According to another embodiment, the pharmaceutically acceptable carrier comprises less than 2.4% hyaluronic acid. According to another embodiment, the pharmaceutically acceptable carrier comprises less than 2.5% hyaluronic acid. According to another embodiment, the pharmaceutically acceptable carrier comprises less than 2.6% hyaluronic acid. According to another embodiment, the pharmaceutically acceptable carrier comprises less than 2.7% hyaluronic acid. According to another embodiment, the pharmaceutically acceptable carrier comprises less than 2.8% hyaluronic acid. According to another embodiment, the pharmaceutically acceptable carrier comprises less than 2.9% hyaluronic acid. According to another embodiment, the pharmaceutically acceptable carrier comprises less than 3.0% hyaluronic acid. According to another embodiment, the pharmaceutically acceptable carrier comprises less than 3.5% hyaluronic acid. According to another embodiment, the pharmaceutically acceptable carrier comprises less than 4.0% hyaluronic acid. According to another embodiment, the pharmaceutically acceptable carrier comprises less than 4.5% hyaluronic acid. According to another embodiment, the pharmaceutically acceptable carrier comprises less than 5.0% hyaluronic acid.

According to some embodiments, the pharmaceutically acceptable carrier includes, but is not limited to, a gel, slow-release solid or semisolid compound, optionally as a sustained release gel. According to some such embodiments, the therapeutic agent is embedded into the pharmaceutically acceptable carrier. According to some embodiments, the therapeutic agent is coated on the pharmaceutically acceptable carrier. The coating can be of any desired material, preferably a polymer or mixture of different polymers. Optionally, the polymer can be utilized during the granulation stage to form a matrix with the active ingredient so as to obtain a desired release pattern of the active ingredient. The gel, slow-release solid or semisolid compound is capable of releasing the active agent over a desired period of time. The gel, slow-release solid or semisolid compound can be implanted in a tissue, including but not limited to the eye, or in close proximity to a blood vessel.

According to another embodiment, the pharmaceutically acceptable carrier comprises a slow-release solid compound. According to one such embodiment, the therapeutic agent is embedded in the slow-release solid compound or coated on the slow-release solid compound. According to yet another embodiment, the pharmaceutically acceptable carrier comprises a slow-release microparticle containing therapeutic agent.

According to another embodiment, the pharmaceutically acceptable carrier is a gel compound, such as a biodegradable hydrogel.

### Particulate Formulation

According to some embodiments, the therapeutic agent is provided in the form of a particle. The term “particle” as used herein refers to nano or microparticles (or in some instances smaller or larger) that may contain in whole or in part the calcium channel antagonist. According to some embodiments, the particulate formulation comprises a plurality of particles impregnated with therapeutic agent. According to one embodiment, the therapeutic agent is contained within the core of the particle surrounded by a coating. According to another embodiment, the therapeutic agent is dispersed throughout the surface of the particle. According to another embodiment, the therapeutic agent is disposed on or in the particle. According to another embodiment, the therapeutic agent is disposed throughout the surface of the particle. According to another embodiment, the therapeutic agent is adsorbed into the particle.

According to some such embodiments, the microparticles are of uniform size distribution. According to some embodiments, the uniform distribution of microparticle size is achieved by a homogenization process to form a uniform emulsion comprising microparticles. According to some such embodiments, each microparticle comprises a matrix. According to some embodiments, the matrix comprises therapeutic agent.

According to some embodiments, the pharmaceutical composition is flowable. According to some embodiments, the particulate formulation component of the pharmaceutical composition is flowable.

According to some embodiments, the particle is selected from the group consisting of a zero order release, first order release, second order release, delayed release, sustained release, immediate release, and a combination thereof. The particle can include, in addition to therapeutic agent(s), any of those materials routinely used in the art of pharmacy and medicine, including, but not limited to, erodible, nonerodible, biodegradable, or nonbiodegradable material or combinations thereof.

According to some embodiments, the particle is a microcapsule that contains the therapeutic agent in a solution or in a semi-solid state. According to some embodiments, the particle is contains the therapeutic agent, in whole or in part. According to some embodiments, the particle is a nanoparticle that contains the therapeutic agent, in whole or in part. According to some embodiments, the particles can be of virtually any shape.

According to some embodiments, the particle size is at least 50 nm. According to some embodiments, the particle size is at least 100 nm. According to some embodiments, the particle size is at least 500 nm. According to some embodiments, the particle size is at least about 1 μm. According to some embodiments, the particle size is at least about 5 μm. According to some embodiments, the particle size is at least about 10 μm. According to some embodiments, the particle size is at least about 15 μm. According to some embodiments, the particle size is at least about 20 μm. According to one embodiment, the particle size is at least about 25 μm. According to another embodiment, the particle size is at least about 30 μm. According to another embodiment, the particle size is at least about 35 μm. According to another embodiment, the particle size is at least about 40 μm. According to another embodiment, the particle size is at least about 45 μm. According to another embodiment, the particle size is at least about 50 μm. According to another embodiment, the particle size is at least about 55 μm. According to another embodiment, the particle size is at least about 60 μm. According to another embodiment, the particle size is at least about 65 μm. According to another embodiment, the particle size is at least about 70 μm. According to another embodiment, the particle size is at least about 75 μm. According to another embodiment, the particle size is at least about 80 μm. According to another embodiment, the particle size is at least about 85 μm. According to another embodiment, the particle size is at least about 90 μm. According to another embodiment, the particle size is at least about 95 μm. According to another embodiment, the particle size is at least about 100 μm.

According to another embodiment, the therapeutic agent can be provided in form of a string. The string can contain the therapeutic agent in a core surrounded by a coating, or therapeutic agent can be dispersed throughout the string, or therapeutic agent(s) may be absorbed into the string. The string can be of any order release kinetics, including zero order release, first order release, second order release, delayed release, sustained release, immediate release, etc., and any combination thereof. The string can include, in addition to therapeutic agent(s), any of those materials routinely used in the art of pharmacy and medicine, including, but not limited to, erodible, nonerodible, biodegradable, or nonbiodegradable material or combinations thereof.

According to another embodiment, the therapeutic agent can be provided in form of a sheet. The sheet can contain the therapeutic agent and optionally an additional therapeutic agent in a core surrounded by a coating, or therapeutic agent and optionally an additional therapeutic agent can be dispersed throughout the sheet, or therapeutic agent can be absorbed into the sheet. The sheet can be of any order release kinetics, including zero order release, first order release, second order release, delayed release, sustained release, immediate release, etc., and any combination thereof. The sheet can include, in addition to therapeutic agent and optionally an additional therapeutic agent, any of those materials routinely used in the art of pharmacy and medicine, including, but not limited to, erodible, nonerodible, biodegradable, or nonbiodegradable material or combinations thereof.

According to some embodiments, the pharmaceutical composition further comprises a preservative agent. According to some such embodiments, the pharmaceutical composition is presented in a unit dosage form. Exemplary unit dosage forms include, but are not limited to, ampoules or multi-dose containers.

According to some embodiments, the microparticulate formulation comprises a suspension of microparticles. According to some embodiments, the pharmaceutical composition further comprises at least one of a suspending agent, a stabilizing agent and a dispersing agent. According to some such embodiments, the pharmaceutical composition is presented as a suspension. According to some such embodiments, the pharmaceutical composition is presented as a solution. According to some such embodiments, the pharmaceutical composition is presented as an emulsion.

According to some embodiments, a formulation of the pharmaceutical composition comprises an aqueous solution of therapeutic agent in water-soluble form. According to some embodiments, the formulation of the pharmaceutical composition comprises an oily suspension of therapeutic agent. Oily suspension of therapeutic agent can be prepared using suitable lipophilic solvents. Exemplary lipophilic solvents or vehicles include, but are not limited to, fatty oils such as sesame oil, or synthetic fatty acid esters, such as ethyl oleate or triglycerides. According to some embodiments, the formulation of the pharmaceutical composition comprises an aqueous suspension of the therapeutic agent. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the therapeutic agent(s) to allow for the preparation of highly concentrated solutions. Alternatively, the therapeutic agent can be in powder form for constitution with a suitable vehicle, e.g., sterile pyrogen-free water, before use.

Suitable liquid or solid pharmaceutical preparations include, for example, microencapsulated dosage forms, and if appropriate, with one or more excipients, encochleated, coated onto microscopic gold particles, contained in liposomes, pellets for implantation into the tissue, or dried onto an object to be rubbed into the tissue. As used herein, the term “microencapsulation” refers to a process in which very tiny droplets or particles are surrounded or coated with a continuous film of biocompatible, biodegradable, polymeric or non-polymeric material to produce solid structures including, but not limited to, nonpareils, pellets, crystals, agglomerates, microspheres, or nanoparticles. Such pharmaceutical compositions also can be in the form of granules, beads, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, or solubilizers are customarily used as described above. The pharmaceutical compositions are suitable for use in a variety of drug delivery systems. For a brief review of methods for drug delivery, see Langer (1990) Science 249, 1527-1533, which is incorporated herein by reference.

### Microencapsulation Process

Examples of microencapsulation processes and products; methods for the production of emulsion-based microparticles; emulsion-based microparticles and methods for the production thereof; solvent extraction microencapsulation with tunable extraction rates; microencapsulation process with solvent and salt; a continuous double emulsion process for making microparticles; drying methods for tuning microparticle properties, controlled release systems from polymer blends; polymer mixtures comprising polymers having different non-repeating units and methods for making and using the same; and an emulsion based process for preparing microparticles and workhead assembly for use with same are disclosed and described in U.S. Pat. No. 5, 407,609 (entitled Microencapsulation Process and Products Thereof), U.S. application Ser. No. 10/553,003 (entitled Method for the production of emulsion-based microparticles), U.S. application Ser. No. 11/799,700 (entitled Emulsion-based microparticles and methods for the production thereof), U.S. application Ser. No. 12/557,946 (entitled Solvent Extraction Microencapsulation With Tunable Extraction Rates) , U.S. application Ser. No. 12/779,138 (entitled Hyaluronic Acid (HA) Injection Vehicle), U.S. application Ser. No. 12/562,455 entitled Microencapsulation Process With Solvent And Salt) , U.S. application Ser. No. 12/338,488 (entitled Process For Preparing Microparticles Having A Low Residual Solvent Volume); U.S. application Ser. No. 12/692,027 (entitled Controlled Release Systems From Polymer Blends); U.S. application Ser. No. 12/692,020 (entitled Polymer Mixtures Comprising Polymers Having Different Non-Repeating Units And Methods For Making And Using Same); U.S. application Ser. No. 10/565,401 (entitled “Controlled release compositions”); U.S. application Ser. No. 12/692,029 (entitled “Drying Methods for Tuning Microparticle Properties); U.S. application Ser. No. 12/968,708 (entitled “Emulsion Based Process for Preparing Microparticles and Workhead for Use with Same); and U.S. application Ser. No. 13/074,542 (entitled Composition and Methods for Improved Retention of a Pharmaceutical Composition at a Local Administration Site”) The contents of each of these are incorporated herein by reference in its entirety.

According to some embodiments, delivery of therapeutic agent using microparticle technology involves bioresorbable, polymeric particles that encapsulate therapeutic agent and optionally an additional therapeutic agent.

According to one embodiment, the microparticle formulation comprises a polymer matrix, wherein therapeutic agent is impregnated in the polymer matrix. According to one embodiment, the polymer is a slow release polymer. According to one embodiment, the polymer is poly (D, L-Lactide-co-glycolide). According to another embodiment, the polymer is poly(orthoester). According to another embodiment, the polymer is poly(anhydride). According to another embodiment, the polymer is polylactide-polyglycolide.

Both non-biodegradable and biodegradable polymeric materials can be used in the manufacture of particles for delivering the therapeutic agent(s). Such polymers can be natural or synthetic polymers. The polymer is selected based on the period of time over which release is desired. Bioadhesive polymers of particular interest include, but are not limited to, bioerodible hydrogels as described by Sawhney et al in Macromolecules (1993) 26, 581-587, the teachings of which are incorporated herein. Exemplary bioerodible hydrogels include, but are not limited to, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). According to one embodiment, the bioadhesive polymer is hyaluronic acid. According to some such embodiments, the bioadhesive polymer includes less than about 2.3% of hyaluronic acid.

According to some embodiments, the pharmaceutical composition is formulated for parenteral injection, implantation, topical administration, or a combination thereof. According to some such embodiments, the pharmaceutical composition is in the form of a pharmaceutically acceptable sterile aqueous or nonaqueous solution, dispersion, suspension or emulsion or a sterile powder for reconstitution into a sterile injectable solution or dispersion. Examples of suitable aqueous and nonaqueous carriers, diluents, solvents or vehicles include, but are not limited to, water, ethanol, dichloromethane, acetonitrile, ethyl acetate, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Suspensions can further contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

According to some embodiments, the pharmaceutical composition is formulated in an injectable depot form. Injectable depot forms are made by forming microencapsulated matrices of therapeutic agent in a biodegradable polymer. Depending upon the ratio of drug to polymer and the nature of the particular polymer employed, the rate of drug release may be controlled. Such long acting formulations can be formulated with suitable polymeric or hydrophobic materials (for example as an emulsion in an acceptable oil) or ion exchange resins, or as sparingly soluble derivatives, for example, as a sparingly soluble salt. Examples of biodegradable polymers include, but are not limited to, polylactide-polyglycolide, poly(orthoesters) and poly(anhydrides). Depot injectable formulations are also prepared by entrapping the drug in liposomes or microemulsions which are compatible with body tissues.

According to some embodiments, the therapeutic agent is impregnated in or on a polyglycolide (PGA) matrix. PGA is a linear aliphatic polyester developed for use in sutures. Studies have reported PGA copolymers formed with trimethylene carbonate, polylactic acid (PLA), and other polyesters, like polycaprolactone. Some of these copolymers may be formulated as microparticles for sustained drug release.

According to some embodiments, the therapeutic agent is impregnated in or on a polyester—polyethylene glycol matrix. Polyester—polyethylene glycol compounds can be synthesized; these are soft and may be used for drug delivery.

According to some embodiments, the therapeutic agent is impregnated in or on a poly (amino)-derived biopolymer matrix. Poly (amino)-derived biopolymers can include, but are not limited to, those containing lactic acid and lysine as the aliphatic diamine (see, for example, U.S. Pat. No. 5,399,665), and tyrosine-derived polycarbonates and polyacrylates. Modifications of polycarbonates may alter the length of the alkyl chain of the ester (ethyl to octyl), while modifications of polyarylates may further include altering the length of the alkyl chain of the diacid (for example, succinic to sebasic), which allows for a large permutation of polymers and great flexibility in polymer properties.

According to some embodiments, the therapeutic agent is impregnated in or on a polyanhydride matrix. Polyanhydrides are prepared by the dehydration of two diacid molecules by melt polymerization (see, for example, U.S. Pat. No. 4,757,128). These polymers degrade by surface erosion (as compared to polyesters that degrade by bulk erosion). The release of the drug can be controlled by the hydrophilicity of the monomers chosen.

According to some embodiments, the therapeutic agent is impregnated in or on a photopolymerizable biopolymer matrix. Photopolymerizable biopolymers include, but are not limited to, lactic acid/polyethylene glycol/acrylate copolymers.

According to some embodiments, the therapeutic agent is impregnated in or on a hydrogel matrix. The term “hydrogel” refers to a substance resulting in a solid, semisolid, pseudoplastic or plastic structure containing a necessary aqueous component to produce a gelatinous or jelly-like mass. Hydrogels generally comprise a variety of polymers, including hydrophilic polymers, acrylic acid, acrylamide and 2-hydroxyethylmethacrylate (HEMA).

According to some embodiments, the therapeutic agent is impregnated in or on a naturally-occurring biopolymer matrix. Naturally-occurring biopolymers include, but are not limited to, protein polymers, collagen, polysaccharides, and photopolymerizable compounds.

According to some embodiments, the therapeutic agent is impregnated in or on a protein polymer matrix. Protein polymers have been synthesized from self-assembling protein polymers such as, for example, silk fibroin, elastin, collagen, and combinations thereof.

According to some embodiments, the therapeutic agent is impregnated in or on a naturally-occurring polysaccharide matrix. Naturally-occurring polysaccharides include, but are not limited to, chitin and its derivatives, hyaluronic acid, dextran and cellulosics (which generally are not biodegradable without modification), and sucrose acetate isobutyrate (SAIB).

According to some embodiments, the therapeutic agent is impregnated in or on a chitin matrix. Chitin is composed predominantly of 2-acetamido-2-deoxy-D-glucose groups and is found in yeasts, fungi and marine invertebrates (shrimp, crustaceous) where it is a principal component of the exoskeleton. Chitin is not water soluble and the deacetylated chitin, chitosan, only is soluble in acidic solutions (such as, for example, acetic acid). Studies have reported chitin derivatives that are water soluble, very high molecular weight (greater than 2 million daltons), viscoelastic, non-toxic, biocompatible and capable of crosslinking with peroxides, gluteraldehyde, glyoxal and other aldehydes and carbodiamides, to form gels.

According to some embodiments, the therapeutic agent is impregnated in or on a hyaluronic acid (HA) matrix. Hyaluronic acid (HA), which is composed of alternating glucuronidic and glucosaminidic bonds and is found in mammalian vitreous humor, extracellular matrix of the brain, synovial fluid, umbilical cords and rooster combs from which it is isolated and purified, also can be produced by fermentation processes.

According to some embodiments, the pharmaceutical composition further comprises an adjuvant. Exemplary adjuvants include, but are not limited to, preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. Isotonic agents, for example, sugars, sodium chloride and the like, can also be included. Prolonged absorption of the injectable pharmaceutical form can be brought about by the use of agents delaying absorption, for example, aluminum monostearate and gelatin.

The formulations can be sterilized, for example, by terminal gamma irradiation, filtration through a bacterial-retaining filter or by incorporating sterilizing agents in the form of sterile solid compositions that may be dissolved or dispersed in sterile water or other sterile injectable medium just prior to use. Injectable preparations, for example, sterile injectable aqueous or oleaginous suspensions may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol, dichloromethane, ethyl acetate, acetonitrile, etc. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose any bland fixed oil may be employed including synthetic mono- or diglycerides. In addition, fatty acids such as oleic acid are used in the preparation of injectables.

Formulations for parenteral (including but not limited to, subcutaneous, subconjunctival, intraocular, intraorbital, intradermal, intramuscular, intravenous, intraarterial, intrathecal, intraventricular and intraarticular) administration include aqueous and non-aqueous sterile injection solutions that can contain anti-oxidants, buffers, bacteriostats and solutes, which render the formulation isotonic with the blood of the intended recipient; and aqueous and non-aqueous sterile suspensions, which can include suspending agents and thickening agents.

According to another embodiment, the pharmaceutical composition is formulated by conjugating the therapeutic agent to a polymer that enhances aqueous solubility. Examples of suitable polymers include but are not limited to polyethylene glycol, poly-(d-glutamic acid), poly-(1-glutamic acid), poly-(1-glutamic acid), poly-(d-aspartic acid), poly-(1-aspartic acid), poly-(1-aspartic acid) and copolymers thereof. Polyglutamic acids having molecular weights between about 5,000 to about 100,000, with molecular weights between about 20,000 and about 80,000 may be used and with molecular weights between about 30,000 and about 60,000 may also be used. The polymer is conjugated via an ester linkage to one or more hydroxyls of an inventive epothilone using a protocol as essentially described by U.S. Pat. No. 5,977,163 which is incorporated herein by reference. Particular conjugation sites include the hydroxyl off carbon-21 in the case of 21-hydroxy-derivatives of the described invention. Other conjugation sites include, but are not limited, to the hydroxyl off carbon 3 and/or the hydroxyl off carbon 7.

Exemplary buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

According to another embodiment, the semisolid multiparticulate delivery system comprises a partially or in whole biocompatible, biodegradable, viscous semisolid wherein the semisolid comprises a hydrogel, wherein the hydrogel contains the therapeutic agent. The term “hydrogel” as used herein refers to a substance resulting in a solid, semisolid, pseudoplastic, or plastic structure containing a necessary aqueous component to produce a gelatinous or jelly-like mass. The hydrogel incorporates and retains significant amounts of H₂O, which eventually will reach an equilibrium content in the presence of an aqueous environment. According to one embodiment, glyceryl monooleate, hereinafter referred to as GMO, is the intended semisolid delivery system or hydrogel. However, many hydrogels, polymers, hydrocarbon compositions and fatty acid derivatives having similar physical/chemical properties with respect to viscosity/rigidity may function as a semisolid delivery system.

According to one embodiment, the gel system is produced by heating GMO above its melting point (40-50° C.) and by adding a warm aqueous-based buffer or electrolyte solution, such as, for example, phosphate buffer or normal saline, which thus produces a three-dimensional structure. The aqueous-based buffer may be comprised of other aqueous solutions or combinations containing semi-polar solvents.

GMO provides a predominantly lipid-based hydrogel, which has the ability to incorporate lipophilic materials. The term “lipophilic” as used herein refers to preferring or possessing an affinity for a non-polar environment compared to a polar or aqueous environment. GMO further provides internal aqueous channels that incorporate and deliver hydrophilic compounds. The term “hydrophilic” as used herein refers to a material or substance having an affinity for polar substances, such as water. It is recognized that at room temperature (25° C.), the gel system may exhibit differing phases which comprise a broad range of viscosity measures.

According to one embodiment, two gel system phases are utilized due to their properties at room temperature and physiologic temperature (about 37° C.) and pH (about 7.4). Within the two gel system phases, the first phase is a lamellar phase of approximately 5% to approximately 15% H₂O content and approximately 95% to approximately 85% GMO content. The lamellar phase is a moderately viscous fluid, that may be easily manipulated, poured and injected. The second phase is a cubic phase consisting of approximately 15% to approximately 40% H₂O content and approximately 85%-60% GMO content. It has an equilibrium water content of approximately 35% to approximately 40% by weight. The term “equilibrium water content” as used herein refers to maximum water content in the presence of excess water. Thus the cubic phase incorporates water at approximately 35% to approximately 40% by weight. The cubic phase is highly viscous. The viscosity exceeds 1.2 million centipoise (cp) when measured by a Brookfield viscometer; where 1.2 million cp is the maximum measure of viscosity obtainable via the cup and bob configuration of the Brookfield viscometer. According to some such embodiments, a therapeutic agent may be incorporated into the semisolid so as to provide a system for sustained, continuous delivery. According to some such embodiments, the therapeutic agent is a calcium channel antagonist. According to some such embodiments, the therapeutic agent is a dihydropyridine calcium channel antagonist. According to some such embodiments, the therapeutic agent is nimodipine. According to some such embodiments, other therapeutic agents, biologically-active agents, drugs, medicaments and inactives may be incorporated into the semisolid for providing a local biological, physiological, or therapeutic effect in the body at various release rates.

According to some embodiments, alternative semisolids, modified formulations and methods of production are utilized such that the lipophilic nature of the semisolid is altered, or in the alternative, the aqueous channels contained within the semisolid are altered. Thus, various therapeutic agents in varying concentrations may diffuse from the semisolid at differing rates, or be released therefrom over time via the aqueous channels of the semisolid. Hydrophilic substances may be utilized to alter semisolid consistency or therapeutic agent release by alteration of viscosity, fluidity, surface tension or the polarity of the aqueous component. For example, glyceryl monostearate (GMS), which is structurally identical to GMO with the exception of a double bond at Carbon 9 and Carbon 10 of the fatty acid moiety rather than a single bond, does not gel upon heating and the addition of an aqueous component, as does GMO. However, because GMS is a surfactant, GMS is miscible in H₂O up to approximately 20% weight/weight. The term “surfactant” as used herein refers to a surface active agent that is miscible in H₂O in limited concentrations as well as polar substances. Upon heating and stirring, the 80% H₂O/20% GMS combination produces a spreadable paste having a consistency resembling hand lotion. The paste then is combined with melted GMO so as to form the cubic phase gel possessing a high viscosity referred to above.

According to another embodiment, hydrolyzed gelatin, such as commercially available Gelfoam™, is utilized for altering the aqueous component. Approximately 6.25% to 12.50% concentration of Gelfoam™ by weight may be placed in approximately 93.75% to 87.50% respectively by weight H₂O or another aqueous based buffer. Upon heating and stirring, the H₂O (or other aqueous buffer)/Gelfoam™ combination produces a thick gelatinous substance. The resulting substance is combined with GMO; a product so formed swells and forms a highly viscous, translucent gel being less malleable in comparison to neat GMO gel alone.

According to another embodiment, polyethylene glycols (PEG's) can be utilized for altering the aqueous component to aid in drug solubilization. Approximately 0.5% to 40% concentration of PEG's (depending on PEG molecular weight) by weight can be placed in approximately 99.5% to 60% H2O or other aqueous based buffer by weight. Upon heating and stirring, the H2O (or other aqueous buffer)/PEG combination produces a viscous liquid to a semisolid substance. The resulting substance is combined with GMO, whereby a product so formed swells and forms a highly viscous gel.

According to some embodiments, the therapeutic agent releases from the semisolid through diffusion, conceivably in a biphasic manner. A first phase involves, for example, a lipophilic drug contained within the lipophilic membrane that diffuses therefrom into the aqueous channel. The second phase involves diffusion of the drug from the aqueous channel into the external environment. Being lipophilic, the drug may orient itself inside the GMO gel within its proposed lipid bi-layer structure. Thus, incorporating greater than approximately 7.5% of the drug by weight into GMO causes a loss of the integrity of the three-dimensional structure whereby the gel system no longer maintains the semisolid cubic phase, and reverts to the viscous lamellar phase liquid. According to some such embodiments, the therapeutic agent is nimodipine. According to some such embodiments, the therapeutic agent is a calcium channel antagonist. According to some such embodiments, the therapeutic agent is an endothelin receptor antagonist. According to another embodiment, about 1% to about 45% of therapeutic agent is incorporated by weight into a GMO gel at physiologic temperature without disruption of the normal three-dimensional structure. As a result, this system allows the ability of significantly increased flexibility with drug dosages. Because the delivery system is malleable, it may be delivered and manipulated in an implant site, for example, adjacent to cerebral arteries or the subarachnoid space, so as to adhere and conform to contours of walls, spaces, or other voids in the body as well as completely fill all voids existing. The delivery system ensures drug distribution and uniform drug delivery throughout the implant site. Ease of delivery and manipulation of the delivery system within a space, for example, but not limited to the subarachnoid space, is facilitated via a semisolid delivery apparatus. A semisolid delivery apparatus facilitates targeted and controlled delivery of the delivery system.

Alternatively, the described invention provides a semisolid delivery system, which acts as a vehicle for local delivery of therapeutic agents, comprising a lipophilic, hydrophilic or amphophilic, solid or semisolid substance, heated above its melting point and thereafter followed by inclusion of a warm aqueous component so as to produce a gelatinous composition of variable viscosity based on water content. Therapeutic agent(s) is/are incorporated and dispersed into the melted lipophilic component or the aqueous buffer component prior to mixing and formation of the semisolid system. The gelatinous composition is placed within the semisolid delivery apparatus for subsequent placement, or deposition. Being malleable, the gel system is easily delivered and manipulated via the semisolid delivery apparatus in an implant site, where it adheres and conforms to contours of the implantation site, spaces, or other voids in the body as well as completely filling all voids existing. Alternatively, a multiparticulate component, comprised of a biocompatible polymeric or non-polymeric system is utilized for producing microspheres having a therapeutic agent entrapped therein. Following final processing methods, the microspheres are incorporated into the semisolid system and subsequently placed within the semisolid delivery apparatus so as to be easily delivered therefrom into an implant site or comparable space, whereby therapeutic agent is subsequently released therefrom by (a) drug release mechanism(s).

### Methods

According to another aspect, the described invention provides a method for treating one or more adverse consequences of abnormal intraocular pressure, retinal vascular disease and retinal ganglion cell death in order to reduce visual loss in a mammal in need thereof, the method comprising:

(a) providing a pharmaceutical composition comprising
(i) a particulate formulation of a therapeutic agent; and optionally
(ii) a pharmaceutically acceptable carrier;
the pharmaceutical composition being characterized by:
dispersal of the therapeutic agent (e.g., the voltage-gated calcium channel antagonist, the endothelin receptor antagonist, or the combination thereof) throughout each particle, adsorption of the therapeutic agent (e.g., the voltage-gated calcium channel antagonist, the endothelin receptor antagonist, or the combination thereof, and optionally an additional therapeutic agent) onto the particles, or placement of the therapeutic agent (e.g., the voltage-gated calcium channel antagonist, the endothelin receptor antagonist, or the combination thereof, and optionally an additional therapeutic agent) in a core surrounded by a coating,
sustained release of the therapeutic agent (e.g., voltage-gated calcium channel antagonist, the endothelin receptor antagonist, or the combination thereof, and optionally an additional therapeutic agent) from the composition, and
a local therapeutic effect that is effective to reduce signs or symptoms of the one or more adverse consequence of an eye disease, including abnormal intraocular pressure, retinal vascular disease, and retinal ganglion cell death, without entering systemic circulation in an amount to cause unwanted side effects; and
   (b) administering a therapeutic amount of the pharmaceutical composition by a means for administering the therapeutic amount of the pharmaceutical composition at a site of administration.

According to some embodiments, the administering of the pharmaceutical composition can be, for example, into the eye, into the orbit, or into the subconjunctival space. According to some embodiments, the administering into the eye comprises administering into the vitreous humor, the aqueous humor, or both.

According to one embodiment, a means for administering therapeutic amount of the pharmaceutical composition in step (b) includes, but is not limited to, injection, a catheter, a punctual plug, a polymerized collagen gel, a contact lens and the like.

Non-limiting examples of contact lenses include a soft contact lens, a gas permeable lens and a hybrid contact lens.

Soft contacts are made of hydrophilic plastic polymers called hydrogels. These materials can absorb water and become soft and pliable without losing their optical qualities.

Soft contacts—including new highly oxygen-permeable varieties called silicone hydrogel lenses—can be made with either a lathe cutting process or an injection molding process.

In the lathe cutting process, non-hydrated disks (or “buttons”) of soft contact lens material are individually mounted on spinning shafts and are shaped with computer-controlled precision cutting tools. After the front and back surfaces are shaped with the cutting tool, the lens is then removed from the lathe and hydrated to soften it. The finished lenses then undergo quality assurance testing. Though the lathe cutting process has more steps and is more time-consuming than an injection molding process, over the years the process has become more automated. With computers and industrial robotics, it now takes only a few minutes to create a lathe-cut soft contact lens.

In the injection molding process, the soft contact lens material is heated to a molten state and is then injected into computer-designed molds under pressure. The lenses are then quickly cooled and removed from the molds. The edges of the lenses are polished smooth, and the lenses are hydrated to soften them prior to undergoing quality assurance testing. Most disposable contact lenses are made with an injection molding process, as this method is faster and less expensive than lathe cutting processes.

Most rigid gas permeable lenses (RGP or GP lenses) are made of oxygen-permeable plastic polymers containing silicone and fluorine. GP lenses contain very little water and remain rigid on the eye. Gas permeable lenses are custom-made to specifications supplied by the prescribing doctor and hence are more costly than mass-produced soft lenses. A greater degree of customization is needed for GP contacts because they maintain their shape and do not conform to the eye like soft lenses. Minute differences in lens design can be the difference between a comfortable fit and contact lens failure with gas permeable lenses. GP contacts are made with a computerized precision lathe cutting process similar to that used for lathe-cut soft lenses. These lenses are shipped dry to the prescribing doctor. The doctor's office then soaks the lenses in a GP contact lens care solution prior to dispensation to the patient. This solution “conditions” the lens surfaces for greater wearing comfort.

Hybrid contact lenses have a central optical zone made of rigid gas permeable plastic, surrounded by a peripheral fitting zone made of a soft contact lens material. Hybrid lenses are made with a process very similar to lathe-cut soft contact lenses, with one very significant difference: the plastic disks cut with the lathe have a GP center, surrounded by non-hydrated soft contact lens material. The two materials are bonded together with proprietary technology to prevent separation of the materials after the lenses are cut and hydrated.

According to some embodiments, the retinal vascular disease is a result of an underlying condition. Exemplary underlying conditions include, but are not limited to, an aneurysm, a vascular blockage, an ischemia, diabetes and the like.

According to some embodiments, the pharmaceutical composition, when administered in a therapeutic amount at a site of delivery in the mammal, is effective in preventing or reducing the incidence of one or more adverse consequence of an eye disease, including abnormal intraocular pressure, retinal vascular disease and retinal ganglion cell death. According to another embodiment, the retinal vascular disease includes, but is not limited to, a vascular blockage, a diabetic retinopathy, an ocular ischemic syndrome and glaucoma. According to another embodiment, the site of delivery is in close proximity to a blood vessel that is contributing to the one or more adverse consequence of an eye disease, including abnormal intraocular pressure, retinal vascular disease and retinal ganglion cell death. According to one embodiment, the site of delivery is within a blood vessel that is contributing to the one or more adverse consequence of an eye disease, including abnormal intraocular pressure, retinal vascular disease and retinal ganglion cell death.

According to some embodiments, the pharmaceutical composition is administered, for example, topically, parenterally or by implantation.

According to some embodiments, the site of delivery is within a blood vessel 10 mm, less than 10 mm, less than 9.9 mm, less than 9.8 mm, less than 9.7 mm, less than 9.6 mm, less than 9.5 mm, less than 9.4 mm, less than 9.3 mm, less than 9.2 mm, less than 9.1 mm, less than 9.0 mm, less than 8.9 mm, less than 8.8 mm, less than 8.7 mm, less than 8.6 mm, less than 8.5 mm, less than 8.4 mm, less than 8.3 mm, less than 8.2 mm, less than 8.1 mm, less than 8.0 mm, less than 7.9 mm, less than 7.8 mm, less than 7.7 mm, less than 7.6 mm, less than 7.5 mm, less than 7.4 mm, less than 7.3 mm, less than 7.2 mm, less than 7.1 mm, less than 7.0 mm, less than 6.9 mm, less than 6.8 mm, less than 6.7 mm, less than 6.6 mm, less than 6.5 mm, less than 6.4 mm, less than 6.3 mm, less than 6.2 mm, less than 6.1 mm, less than 6.0 mm, less than 5.9 mm, less than 5.8 mm, less than 5.7 mm, less than 5.6 mm, less than 5.5 mm, less than 5.4 mm, less than 5.3 mm, less than 5.2 mm, less than 5.1 mm, less than 5.0 mm, less than 4.9 mm, less than 4.8 mm, less than 4.7 mm, less than 4.6 mm, less than 4.5 mm, less than 4.4 mm, less than 4.3 mm, less than 4.2 mm, less than 4.1 mm, less than 4.0 mm, less than 3.9 mm, less than 3.8 mm, less than 3.7 mm, less than 3.6 mm, less than 3.5 mm, less than 3.4 mm, less than 3.3 mm, less than 3.2 mm, less than 3.1 mm, less than 3.0 mm, less than 2.9 mm, less than 2.8 mm, less than 2.7 mm, less than 2.6 mm, less than 2.5 mm, less than 2.4 mm, less than 2.3 mm, less than 2.2 mm, less than 2.1 mm, less than 2.0 mm, less than 1.9 mm, less than 1.8 mm, less than 1.7 mm, less than 1.6 mm, less than 1.5 mm, less than 1.4 mm, less than 1.3 mm, less than 1.2 mm, less than 1.1 mm, less than 1.0 mm, less than 0.9 mm, less than 0.8 mm, less than 0.7 mm, less than 0.6 mm, less than 0.5 mm, less than 0.4 mm, less than 0.3 mm, less than 0.2 mm, less than 0.1 mm, less than 0.09 mm, less than 0.08 mm, less than 0.07 mm, less than 0.06 mm, less than 0.05 mm, less than 0.04 mm, less than 0.03 mm, less than 0.02 mm, less than 0.01 mm, less than 0.009 mm, less than 0.008 mm, less than 0.007 mm, less than 0.006 mm, less than 0.005 mm, less than 0.004 mm, less than 0.003 mm, less than 0.002 mm, less than 0.001 mm of the blood vessel contributing to the retinal vascular disease.

According to some embodiments, the pharmaceutical composition is capable of releasing the therapeutic agent at the site of delivery within a half-life ranging from 1 day to 180 days. According to one embodiment, the pharmaceutical composition is capable of releasing the therapeutic agent at the site of delivery within a half-life of 1 day. According to another embodiment, the pharmaceutical composition is capable of releasing the therapeutic agent at the site of delivery within a half-life of 2 days. According to another embodiment, the pharmaceutical composition is capable of releasing the therapeutic agent at the site of delivery within a half-life of 3 days. According to another embodiment, the pharmaceutical composition is capable of releasing the therapeutic agent within a half-life of 4 days. According to another embodiment, the pharmaceutical composition is capable of releasing the therapeutic agent at the site of delivery within a half-life of 5 days. According to another embodiment, the pharmaceutical composition is capable of releasing the therapeutic agent at the site of delivery within a half-life of 6 days. According to another embodiment, the pharmaceutical composition is capable of releasing the therapeutic agent at the site of delivery within a half-life of 7 days. According to another embodiment, the pharmaceutical composition is capable of releasing the therapeutic agent at the site of delivery within a half-life of 8 days. According to another embodiment, the pharmaceutical composition is capable of releasing the therapeutic agent at the site of delivery within a half-life of 9 days. According to another embodiment, the pharmaceutical composition is capable of releasing the therapeutic agent at the site of delivery within a half-life of 10 days. According to another embodiment, the pharmaceutical composition is capable of releasing the therapeutic agent at the site of delivery within a half-life of 15 days. According to another embodiment, the controlled release system is capable of releasing the therapeutic agent at the site of delivery within a half-life of 20 days. According to another embodiment, the controlled release system is capable of releasing the therapeutic agent at the site of delivery within a half-life of 25 days. According to another embodiment, the pharmaceutical composition is capable of releasing the therapeutic agent at the site of delivery within a half-life of 30 days. According to another embodiment, the controlled release system is capable of releasing the therapeutic agent at the site of delivery within a half-life of 35 days. According to another embodiment, the controlled release system is capable of releasing the therapeutic agent at the site of delivery within a half-life of 40 days. According to another embodiment, the controlled release system is capable of releasing the therapeutic agent at the site of delivery within a half-life of 45 days. According to another embodiment, the controlled release system is capable of releasing the therapeutic agent at the site of delivery within a half-life of 50 days. According to another embodiment, the controlled release system is capable of releasing the therapeutic agent at the site of delivery within a half-life of 55 days. According to another embodiment, the controlled release system is capable of releasing the therapeutic agent at the site of delivery within a half-life of 60 days. According to another embodiment, the controlled release system is capable of releasing the therapeutic agent at the site of delivery within a half-life of 75 days. According to another embodiment, the controlled release system is capable of releasing the therapeutic agent at the site of delivery within a half-life of 90 days. According to another embodiment, the controlled release system is capable of releasing the therapeutic agent at the site of delivery within a half-life of 120 days. According to another embodiment, the controlled release system is capable of releasing the therapeutic agent at the site of delivery within a half-life of 150 days. According to another embodiment, the controlled release system is capable of releasing the therapeutic agent at the site of delivery within a half-life of 180 days.

### Therapeutic Effect

According to another embodiment, implantation of the pharmaceutical composition in close proximity to a site of vascular insufficiency contributing to a retinal vascular disease can result in, for example, increased ocular blood flow, increased optic nerve head blood flow as compared to a control, increased choroidal blood flow (CHBF) as compared to a control, increase in ocular fundus pulsation amplitude (FPA) as compared to a control, increased color contrast sensitivity (CCS) as compared to a control, decreased intraocular pressure (TOP) as compared to a control, or a combination thereof.

According to another embodiment, the release of the therapeutic agent at the site of delivery can produce a predominantly localized pharmacologic effect over a desired amount of time. According to one embodiment, the release of the therapeutic agent at the site of delivery produces a predominantly localized pharmacologic effect for 1 day. According to one embodiment, the release of the therapeutic agent at the site of delivery produces a predominantly localized pharmacologic effect for 2 days. According to one embodiment, the release of the therapeutic agent at the site of delivery produces a localized pharmacologic effect for 3 days. According to one embodiment, the release of the therapeutic agent at the site of delivery produces a predominantly localized pharmacologic effect for 4 days. According to one embodiment, the release of the therapeutic agent at the site of delivery produces a predominantly localized pharmacologic effect for 5 days. According to one embodiment, the release of the therapeutic agent at the site of delivery produces a predominantly localized pharmacologic effect for 6 days. According to one embodiment, the release of the therapeutic agent at the site of delivery produces a predominantly localized pharmacologic effect for 7 days. According to one embodiment, the release of the therapeutic agent at the site of delivery produces a predominantly localized pharmacologic effect for 8 days. According to one embodiment, the release of the therapeutic agent at the site of delivery produces a predominantly localized pharmacologic effect for 9 days. According to one embodiment, the release of the therapeutic agent at the site of delivery produces a predominantly localized pharmacologic effect for 10 days. According to one embodiment, the release of the therapeutic agent at the site of delivery produces a predominantly localized pharmacologic effect for 15 days. According to one embodiment, the release of the therapeutic agent at the site of delivery produces a predominantly localized pharmacologic effect for 20 days. According to one embodiment, the release of the therapeutic agent at the site of delivery produces a predominantly localized pharmacologic effect for 25 days. According to one embodiment, the release of the therapeutic agent at the site of delivery produces a predominantly localized pharmacologic effect for 30 days. According to one embodiment, the release of the therapeutic agent at the site of delivery produces a predominantly localized pharmacologic effect for 35 days. According to one embodiment, the release of the therapeutic agent at the site of delivery produces a predominantly localized pharmacologic effect for 40 days. According to one embodiment, the release of the therapeutic agent at the site of delivery produces a predominantly localized pharmacologic effect for 45 days. According to one embodiment, the release of the therapeutic agent at the site of delivery produces a predominantly localized pharmacologic effect for 50 days. According to one embodiment, the release of the therapeutic agent at the site of delivery produces a predominantly localized pharmacologic effect for 55 days. According to one embodiment, the release of the therapeutic agent at the site of delivery produces a predominantly localized pharmacologic effect for 60 days. According to one embodiment, the release of the therapeutic agent at the site of delivery produces a predominantly localized pharmacologic effect for 75 days. According to one embodiment, the release of the therapeutic agent at the site of delivery produces a predominantly localized pharmacologic effect for 90 days. According to one embodiment, the release of the therapeutic agent at the site of delivery produces a predominantly localized pharmacologic effect for 120 days. According to one embodiment, the release of the therapeutic agent at the site of delivery produces a predominantly localized pharmacologic effect for 150 days. According to one embodiment, the release of the therapeutic agent at the site of delivery produces a predominantly localized pharmacologic effect for 180 days.

According to another embodiment, the release of the therapeutic agent at the site of delivery produces a diffuse pharmacologic effect throughout the eye over a desired amount of time. According to another embodiment, the release of the therapeutic agent can produce a diffuse pharmacologic effect throughout the eye for at least 1 day. According to another embodiment, the release of the therapeutic agent can produce a diffuse pharmacologic effect throughout the eye for at least 2 days. According to another embodiment, the release of the therapeutic agent can produce a diffuse pharmacologic effect throughout the eye for at least 3 days. According to another embodiment, the release of the therapeutic agent can produce a diffuse pharmacologic effect throughout the eye for at least 4 days. According to another embodiment, the release of the therapeutic agent can produce a diffuse pharmacologic effect throughout the eye for at least 5 days. According to another embodiment, the release of the therapeutic agent can produce a diffuse pharmacologic effect throughout the eye for at least 6 days. According to another embodiment, the release of the therapeutic agent can produce a diffuse pharmacologic effect throughout the eye for at least 7 days. According to another embodiment, the release of the therapeutic agent can produce a diffuse pharmacologic effect throughout the eye for at least 8 days. According to another embodiment, the release of the therapeutic agent can produce a diffuse pharmacologic effect throughout the eye for at least for 15 days. According to another embodiment, the release of the therapeutic agent can produce a diffuse pharmacologic effect throughout the eye for at least 30 days. According to another embodiment, the release of the therapeutic agent can produce a diffuse pharmacologic effect throughout the eye for at least 35 days. According to another embodiment, the release of the therapeutic agent can produce a diffuse pharmacologic effect throughout the eye for at least 40 days. According to another embodiment, the release of the therapeutic agent can produce a diffuse pharmacologic effect throughout the eye for at least 45 days. According to another embodiment, the release of the therapeutic agent can produce a diffuse pharmacologic effect throughout the eye for at least 50 days. According to another embodiment, the release of the therapeutic agent can produce a diffuse pharmacologic effect throughout the eye for at least 55 days. According to another embodiment, the release of the therapeutic agent can produce a diffuse pharmacologic effect throughout the eye for at least 60 days. According to another embodiment, the release of the therapeutic agent can produce a diffuse pharmacologic effect throughout the eye for at least 60 days. According to another embodiment, the release of the therapeutic agent can produce a diffuse pharmacologic effect throughout the eye for at least 75 days. According to another embodiment, the release of the therapeutic agent can produce a diffuse pharmacologic effect throughout the eye for at least 90 days. According to another embodiment, the release of the therapeutic agent can produce a diffuse pharmacologic effect throughout the eye for at least 120 days.

According to another embodiment, the release of the therapeutic agent can produce a diffuse pharmacologic effect throughout the eye for at least 150 days. According to another embodiment, the release of the therapeutic agent can produce a diffuse pharmacologic effect throughout the eye for at least 180 days.

According to one embodiment, the localized pharmacologic effect at the site of delivery includes, but is not limited to, an increased ocular blood flow as compared to a control, increased optic nerve head blood flow (ONHBF) as compared to a control, increased choroidal blood flow (CHBF) as compared to a control, increase in ocular fundus pulsation amplitude (FPA) as compared to a control, increased color contrast sensitivity (CCS) as compared to a control, decreased intraocular pressure (TOP) as compared to a control, or a combination thereof.

According to one embodiment, the diffuse pharmacologic effect is a reduction of vasospasm such that internal diameter of a blood vessel that is within at least 10 mm, at least 9.9 mm, at least 9.8 mm, at least 9.7 mm, at least 9.6 mm, at least 9.5 mm, at least 9.4 mm, at least 9.3 mm, at least 9.2 mm, at least 9.1 mm, at least 9.0 mm, at least 8.9 mm, at least 8.8 mm, at least 8.7 mm, at least 8.6 mm, at least 8.5 mm, at least 8.4 mm, at least 8.3 mm, at least 8.2 mm, at least 8.1 mm, at least 8.0 mm, at least 7.9 mm, at least 7.8 mm, at least 7.7 mm, at least 7.6 mm, at least 7.5 mm, at least 7.4 mm, at least 7.3 mm, at least 7.2 mm, at least 7.1 mm, at least 7.0 mm, at least 6.9 mm, at least 6.8 mm, at least 6.7 mm, at least 6.6 mm, at least 6.5 mm, at least 6.4 mm, at least 6.3 mm, at least 6.2 mm, at least 6.1 mm, at least 6.0 mm, at least 5.9 mm, at least 5.8 mm, at least 5.7 mm, at least 5.6 mm, at least 5.5 mm, at least 5.4 mm, at least 5.3 mm, at least 5.2 mm, at least 5.1 mm, at least 5.0 mm from the site of delivery is increased as compared to a control.

### Voltage-Gated Calcium Channel Antagonist

Non-limiting examples of the voltage-gated calcium channel antagonist that can be formulated into the composition include, but are not limited to, L-type voltage-gated calcium channel antagonist, N-type voltage-gated calcium channel antagonist, P/Q-type voltage-gated calcium channel antagonist, or a combination thereof.

For example, L-type voltage-gated calcium channel antagonists include, but are not limited to: dihydropyridine L-type antagonists such as nisoldipine, nicardipine, nilvidipine and nifedipine, AHF (such as 4aR,9aS)-(+)-4a-Amino-1,2,3,4,4a,9a-hexahydro-4aH-fluorene, HCl), isradipine (such as 4-(4-Benzofurazanyl)-1,-4-dihydro-2,6-dimethyl-3,5-pyridinedicarboxylic acid methyl 1-methhylethyl ester), calciseptine (such as isolated from (Dendroaspis polylepis ploylepis), H-Arg-Ile-Cys-Tyr-Ile-His-Lys-Ala-Ser-Leu-Pro-Arg-Ala-Thr-Lys-Thr-Cys-Val-Glu-Asn-Thr-Cys-Tyr-Lys-Met-Phe-Ile-Arg-Thr-Gln-Arg-Glu-Tyr-Ile-Ser-Glu-Arg-Gly-Cys-Gly-Cys-Pro-Thr-Ala-Met-Trp- Pro-Tyr-Gln-Thr-Glu-Cys-Cys-Lys-Gly-Asp-Arg-Cys-Asn-Lys-OH, Calcicludine (such as isolated from Dendroaspis angusticeps (eastern green mamba)),(H-Trp-Gln-Pro-Pro-Trp-Tyr-Cys-Lys-Glu-Pro-Val-Arg-Ile-Gly-Ser-Cys-Lys-Lys-Gln-Phe-Ser-Ser-Phe-Tyr-Phe-Lys-Trp-Thr-Ala-Lys-Lys-Cys-Leu-Pro-Phe-Leu-Phe-Ser-Gly-Cys-Gly-Gly-Asn-Ala-Asn-Arg-Phe-Gln- Thr-Ile-Gly-Glu-Cys-Arg-Lys-Lys-Cys-Leu-Gly-Lys-OH, Cilnidipine (such as also FRP-8653, a dihydropyridine-type inhibitor), Dilantizem (such as (2S,3S)-(+)-cis-3-Acetoxy-5-(2-dimethylaminoethyl)-2,3-dihydro-2-(4-methoxyphenyl)-1,5-benzothiazepin-4(5H)-one hydrochloride), diltiazem (such as benzothiazepin-4(5H)-one, 3-(acetyloxy)-5-[2-(dimethylamino)ethyl]-2,3-dihydro-2-(4-methoxyphenyl)-,(+)-cis-, monohydrochloride), Felodipine (such as 4-(2,3-Dichlorophenyl)-1,4-dihydro-2,6-dimethyl-3,5-pyridinecarboxylic acid ethyl methyl ester), FS-2 (such as an isolate from Dendroaspis polylepis polylepis venom), FTX-3.3 (such as an isolate from Agelenopsis aperta), Neomycin sulfate (such as C₂₃H₄₆N₆O₁₃·3H₂SO₄), Nicardipine (such as 1,4-Dihydro-2,6-dimethyl-4-(3-nitrophenypmethyl-2-[methyl(phenylmethypa-mino]-3,5-pyridinedicarboxylic acid ethyl ester hydrochloride, also YC-93, Nifedipine (such as 1,4-Dihydro-2,6-dimethyl-4-(2-nitrophenyl)-3,5-pyridinedicarboxylic acid dimethyl ester), Nimodipine (such as 4-Dihydro-2,6-dimethyl-4-(3-nitrophenyl)-3,5-pyridinedicarboxylic acid 2-methoxyethyl 1-methylethyl ester) or (Isopropyl 2-methoxyethyl 1,4-dihydro-2,6-dimethyl-4-(m-nitrophenyl)-3,5-pyridinedicarboxylate), Nitrendipine (such as 1,4-Dihydro-2,6-dimethyl-4-(3-nitrophenyl)-3,5-pyridinedicarboxylic acid ethyl methyl ester), S-Petasin (such as (3 S,4aR,5R,6R)-[2,3,4,4a,5,6,7,8-Octahydro-3-(2-propenyl)-4a,5-dimethyl-2-o-xo-6-naphthyl]Z-.3′-methylthio-1′-propenoate), Phloretin (such as 2′,4′,6′-Trihydroxy-3-(4-hydroxyphenyl)propiophenone, also 3-(4-Hydroxyphenyl)-1-(2,4,6-trihydroxyphenyl)-1-propanone, also b-(4-Hydroxyphenyl)-2,4,6-trihydroxypropiophenone), Protopine (such as C₂₀H₁₉NO₅Cl), SKF-96365 (such as 1-[b-[3-(4-Methoxyphenyl)propoxy]-4-methoxyphenethyl1-1H-imidazole, HCl), Tetrandine (such as 6,6′,7,12-Tetramethoxy-2,2′-dimethylberbaman), (.+-.)-Methoxyverapamil or (+)-Verapamil (such as 54N-(3,4-Dimethoxyphenylethyl)methylamino]-2-(3,4-dimethoxyphenyl)-2-iso-propylvaleronitrile hydrochloride), and (R)-(+)-B ay K8644 (such as R-(+)-1,4-Dihydro-2,6-dimethyl-5-nitro-442-(trifluoromethyl)phenyl1-3-pyridinecarboxylic acid methyl ester). The foregoing examples may be specific to L-type voltage-gated calcium channels or may inhibit a broader range of voltage-gated calcium channels, e.g. N, P/Q, R, and T-type.

According to some embodiments, the voltage-gated calcium channel antagonist is a dihydropyridine calcium channel antagonist. According to one embodiment, the dihydropyridine calcium channel antagonist is nimodipine. According to one embodiment, the nimodipine has a half-life of 7-10 days when formulated as described herein, and appropriate lipid solubility.

According to some embodiments, the therapeutic agent is an isolated molecule. The term “isolated molecule” as used herein refers to a molecule that is substantially pure and is free of other substances with which it is ordinarily found in nature or in vivo systems to an extent practical and appropriate for its intended use.

According to some embodiments, the therapeutic agent is admixed with a pharmaceutically-acceptable carrier in a pharmaceutical preparation. According to some such embodiments, the therapeutic agent comprises only a small percentage by weight of the preparation. According to some embodiments, the therapeutic agent is substantially pure.

### Endothelin Receptor Antagonist

According to some embodiments, ETA-receptor antagonists may include, but are not limited to, A-127722 (non-peptide), ABT-627 (non-peptide), BMS 182874 (non-peptide), BQ-123 (peptide), BQ-153 (peptide), BQ-162 (peptide), BQ-485 (peptide), BQ-518 (peptide), BQ-610 (peptide), EMD-122946 (non-peptide), FR 139317 (peptide), IPI-725 (peptide), L-744453 (non-peptide), LU 127043 (non-peptide), LU 135252 (non-peptide), PABSA (non-peptide), PD 147953 (peptide), PD 151242 (peptide), PD 155080 (non-peptide), PD 156707 (non-peptide), RO 611790 (non-peptide), SB-247083 (non-peptide), clazosentan (non-peptide), atrasentan (non-peptide), sitaxsentan sodium (non-peptide), TA-0201 (non-peptide), TBC 11251 (non-peptide), TTA-386 (peptide), WS-7338B (peptide), ZD-1611 (non-peptide), and aspirin (non-peptide). ETA/B-receptor antagonists may include, but are not limited to, A-182086 (non-peptide), CGS 27830 (non-peptide), CP 170687 (non-peptide), J-104132 (non-peptide), L-751281 (non-peptide), L-754142 (non-peptide), LU 224332 (non-peptide), LU 302872 (non-peptide), PD 142893 (peptide), PD 145065 (peptide), PD 160672 (non-peptide), RO-470203 (bosentan, non-peptide), RO 462005 (non-peptide), RO 470203 (non-peptide), SB 209670 (non-peptide), SB 217242 (non-peptide), and TAK-044 (peptide). ETB-ireceptor antagonists may include, but are not limited to, A-192621 (non-peptide), A-308165 (non-peptide), BQ-788 (peptide), BQ-017 (peptide), IRL 1038 (peptide), IRL 2500 (peptide), PD-161721 (non-peptide), RES 701-1 (peptide), and RO 468443 (peptide).

Additional Therapeutic Agents

According to one embodiment, the particulate pharmaceutical composition further comprises a therapeutic amount of one or more additional therapeutic agent(s). According to some embodiments, the additional therapeutic agent is a prostaglandin analog. According to some such embodiments, the prostaglandin analog is latanoprost. According to some embodiments, the additional therapeutic agent is one or more Rho kinase inhibitor. Exemplary Rho kinase inhibitors include, without limitation, Y-27632 2HCl (R&D Systems Inc., Minneapolis, Minn.), Triazovivin® (StemRD, Burlingame, Calif.), Slx-2119 (MedChem Express, Namiki Shoji Cop., LTD), WF-536 [(+)-®-4-(1-aminoethyl)-N-(4-pyridyl) benzamide monohydrochloride] (Mitsubishi Pharma Corporation, Osaka, Japan), RK1-1447 (University of South Florida, Tampa, Fla., and Moffitt Cancer Center, Tampa, Fla.; Roberta Pireddu et al., “Pyridylthiazole-based ureas as inhibitors of Rho associated protein kinases (ROCK1 and 2).” (2012) Medchemcomm. 3(6):699-709), Fasudil® (Asahi-KASEI Corp., Osaka, Japan), Fasudil® hydrochloride (R&D Systems Inc., Minneapolis, Minn.), GSK429286A (R&D Systems Inc., Minneapolis, Minn.), Rockout® (EMD Millipore, Philadelphia, Pa.), SR 3677 dihydrochloride (R&D Systems Inc., Minneapolis, Minn.); SB 772077B (R&D Systems Inc., Minneapolis, Minn.), AS 1892802 (R&D Systems Inc., Minneapolis, Minn.), H 1152 dihydrochloride (R&D Systems Inc., Minneapolis, Minn.), GSK 269962 (R&D Systems Inc., Minneapolis, Minn.), HA 1100 hydrochloride (R&D Systems Inc., Minneapolis, Minn.), Glycyl-H-1152 dihydrochloride (R&D Systems Inc., Minneapolis, Minn.), AR-12286 (Aerie Pharmaceuticals), AR-13324 (Rhopressa, Aerie Pharmaceuticals), AMA-0076 (Amakem Therapeutics), and K-115 (Kumatomo University, Japan). According to some other embodiments, the additional therapeutic agent includes a combination of a Rho kinase inhibitor and a prostaglandin analog.

Pharmaceutically acceptable carrier

According to some embodiments, the pharmaceutical composition comprises a pharmaceutically acceptable carrier.

According to one embodiment, the pharmaceutically acceptable carrier is a solid carrier or excipient. According to another embodiment, the pharmaceutically acceptable carrier is a gel-phase carrier or excipient. Examples of carriers or excipients include, but are not limited to, calcium carbonate, calcium phosphate, various monomeric and polymeric sugars (including without limitation hyaluronic acid), starches, cellulose derivatives, gelatin, and polymers. An exemplary carrier can also include saline vehicle, e.g. hydroxyl propyl methyl cellulose (HPMC) in phosphate buffered saline (PBS).

According to some embodiments, the pharmaceutically acceptable carrier imparts stickiness. According to one embodiment, the pharmaceutically acceptable carrier comprises hyaluronic acid. According to some embodiments, the pharmaceutically acceptable carrier comprises 0% to 5% hyaluronic acid. According to one embodiment, the pharmaceutically acceptable carrier comprises less than 0.05% hyaluronic acid. According to another embodiment, the pharmaceutically acceptable carrier comprises less than 0.1% hyaluronic acid. According to another embodiment, the pharmaceutically acceptable carrier comprises less than 0.2% hyaluronic acid. According to another embodiment, the pharmaceutically acceptable carrier comprises less than 0.3% hyaluronic acid. According to another embodiment, the pharmaceutically acceptable carrier comprises less than 0.4% hyaluronic acid. According to another embodiment, the pharmaceutically acceptable carrier comprises less than 0.5% hyaluronic acid. According to another embodiment, the pharmaceutically acceptable carrier comprises less than 0.6% hyaluronic acid. According to another embodiment, the pharmaceutically acceptable carrier comprises less than 0.7% hyaluronic acid. According to another embodiment, the pharmaceutically acceptable carrier comprises less than 0.8% hyaluronic acid. According to another embodiment, the pharmaceutically acceptable carrier comprises less than 0.9% hyaluronic acid. According to another embodiment, the pharmaceutically acceptable carrier comprises less than 1.0% hyaluronic acid. According to another embodiment, the pharmaceutically acceptable carrier comprises less than 1.1% hyaluronic acid. According to another embodiment, the pharmaceutically acceptable carrier comprises less than 1.2% hyaluronic acid. According to another embodiment, the pharmaceutically acceptable carrier comprises less than 1.3% hyaluronic acid. According to another embodiment, the pharmaceutically acceptable carrier comprises less than 1.4% hyaluronic acid. According to another embodiment, the pharmaceutically acceptable carrier comprises less than 1.5% hyaluronic acid. According to another embodiment, the pharmaceutically acceptable carrier comprises less than 1.6% hyaluronic acid. According to another embodiment, the pharmaceutically acceptable carrier comprises less than 1.7% hyaluronic acid. According to another embodiment, the pharmaceutically acceptable carrier comprises less than 1.8% hyaluronic acid. According to another embodiment, the pharmaceutically acceptable carrier comprises less than 1.9% hyaluronic acid. According to another embodiment, the pharmaceutically acceptable carrier comprises less than 2.0% hyaluronic acid. According to another embodiment, the pharmaceutically acceptable carrier comprises less than 2.1% hyaluronic acid. According to another embodiment, the pharmaceutically acceptable carrier comprises less than 2.2% hyaluronic acid. According to another embodiment, the pharmaceutically acceptable carrier comprises less than 2.3% hyaluronic acid. According to another embodiment, the pharmaceutically acceptable carrier comprises less than 2.4% hyaluronic acid. According to another embodiment, the pharmaceutically acceptable carrier comprises less than 2.5% hyaluronic acid. According to another embodiment, the pharmaceutically acceptable carrier comprises less than 2.6% hyaluronic acid. According to another embodiment, the pharmaceutically acceptable carrier comprises less than 2.7% hyaluronic acid. According to another embodiment, the pharmaceutically acceptable carrier comprises less than 2.8% hyaluronic acid. According to another embodiment, the pharmaceutically acceptable carrier comprises less than 2.9% hyaluronic acid. According to another embodiment, the pharmaceutically acceptable carrier comprises less than 3.0% hyaluronic acid. According to another embodiment, the pharmaceutically acceptable carrier comprises less than 3.5% hyaluronic acid. According to another embodiment, the pharmaceutically acceptable carrier comprises less than 4.0% hyaluronic acid. According to another embodiment, the pharmaceutically acceptable carrier comprises less than 4.5% hyaluronic acid. According to another embodiment, the pharmaceutically acceptable carrier comprises less than 5.0% hyaluronic acid.

According to some embodiments, the pharmaceutically acceptable carrier includes, but is not limited to, a gel, slow-release solid or semisolid compound, optionally as a sustained release gel. According to some such embodiments, the therapeutic agent is embedded into the pharmaceutically acceptable carrier. According to some embodiments, the therapeutic agent is coated on the pharmaceutically acceptable carrier. The coating can be of any desired material, preferably a polymer or mixture of different polymers. Optionally, the polymer can be utilized during the granulation stage to form a matrix with the active ingredient so as to obtain a desired release pattern of the active ingredient. The gel, slow-release solid or semisolid compound is capable of releasing the active agent over a desired period of time. The gel, slow-release solid or semisolid compound can be implanted in a tissue within human brain, for example, but not limited to, in close proximity to a blood vessel, such as a cerebral artery.

According to another embodiment, the pharmaceutically acceptable carrier comprises a slow-release solid compound. According to one such embodiment, the therapeutic agent is embedded in the slow-release solid compound or coated on the slow-release solid compound. According to yet another embodiment, the pharmaceutically acceptable carrier comprises a slow-release microparticle containing therapeutic agent.

According to another embodiment, the pharmaceutically acceptable carrier is a gel compound, such as a biodegradable hydrogel.

### Particulate Formulation

According to some embodiments, the therapeutic agent is provided in the form of a particle. The term “particle” as used herein refers to nano or microparticles (or in some instances smaller or larger) that may contain in whole or in part the calcium channel antagonist. According to some embodiments, the microparticulate formulation comprises a plurality of particles impregnated with the therapeutic agent. According to one embodiment, the therapeutic agent is contained within the core of the particle surrounded by a coating. According to another embodiment, the therapeutic agent is dispersed throughout the surface of the particle. According to another embodiment, the therapeutic agent is disposed on or in the particle. According to another embodiment, the therapeutic agent is disposed throughout the surface of the particle. According to another embodiment, the therapeutic agent is adsorbed into the particle.

According to some such embodiments, the particles are of uniform size distribution. According to some embodiments, the uniform distribution of microparticle size is achieved by a homogenization process to form a uniform emulsion comprising microparticles. According to some such embodiments, each microparticle comprises a matrix. According to some embodiments, the matrix comprises the therapeutic agent.

According to some embodiments, the pharmaceutical composition is flowable. According to some embodiments, the particulate formulation component of the pharmaceutical composition is flowable.

According to some embodiments, the particle is selected from the group consisting of a zero order release, first order release, second order release, delayed release, sustained release, immediate release, and a combination thereof. The particle can include, in addition to therapeutic agent(s), any of those materials routinely used in the art of pharmacy and medicine, including, but not limited to, erodible, nonerodible, biodegradable, or nonbiodegradable material or combinations thereof.

According to some embodiments, the particle is a microcapsule that contains the therapeutic agent in a solution or in a semi-solid state. According to some embodiments, the particle is a microparticle that contains the therapeutic agent, in whole or in part. According to some embodiments, the particle is a nanoparticle that contains the therapeutic agent, in whole or in part. According to some embodiments, the particles can be of virtually any shape.

According to some embodiments, the particle size is at least 50 nm. According to some embodiments, the particle size is at least 100 nm. According to some embodiments, the particle size is at least 500 nm. According to some embodiments, the particle size is at least about 1 μm. According to some embodiments, the particle size is at least about 5 μm. According to some embodiments, the particle size is at least about 10 μm. According to some embodiments, the particle size is at least about 15 μm. According to some embodiments, the particle size is at least about 20 μm. According to one embodiment, the particle size is at least about 25 μm. According to another embodiment, the particle size is at least about 30 μm. According to another embodiment, the particle size is at least about 35 μm. According to another embodiment, the particle size is at least about 40 μm. According to another embodiment, the particle size is at least about 45 μm. According to another embodiment, the particle size is at least about 50 μm. According to another embodiment, the particle size is at least about 55 μm. According to another embodiment, the particle size is at least about 60 μm. According to another embodiment, the particle size is at least about 65 μm. According to another embodiment, the particle size is at least about 70 μm. According to another embodiment, the particle size is at least about 75 μm. According to another embodiment, the particle size is at least about 80 μm. According to another embodiment, the particle size is at least about 85 μm. According to another embodiment, the particle size is at least about 90 μm. According to another embodiment, the particle size is at least about 95 μm. According to another embodiment, the particle size is at least about 100 μm.

According to another embodiment, the therapeutic agent can be provided in form of a string. The string can contain the therapeutic agent in a core surrounded by a coating, or therapeutic agent can be dispersed throughout the string, or therapeutic agent(s) may be absorbed into the string. The string can be of any order release kinetics, including zero order release, first order release, second order release, delayed release, sustained release, immediate release, etc., and any combination thereof. The string can include, in addition to therapeutic agent(s), any of those materials routinely used in the art of pharmacy and medicine, including, but not limited to, erodible, nonerodible, biodegradable, or nonbiodegradable material or combinations thereof.

According to another embodiment, the therapeutic agent can be provided in form of a sheet. The sheet can contain the therapeutic agent and optionally an additional therapeutic agent in a core surrounded by a coating, or therapeutic agent and optionally an additional therapeutic agent can be dispersed throughout the sheet, or therapeutic agent can be absorbed into the sheet. The sheet can be of any order release kinetics, including zero order release, first order release, second order release, delayed release, sustained release, immediate release, etc., and any combination thereof. The sheet can include, in addition to therapeutic agent and optionally an additional therapeutic agent, any of those materials routinely used in the art of pharmacy and medicine, including, but not limited to, erodible, nonerodible, biodegradable, or nonbiodegradable material or combinations thereof.

According to some embodiments, the pharmaceutical composition further comprises a preservative agent. According to some such embodiments, the pharmaceutical composition is presented in a unit dosage form. Exemplary unit dosage forms include, but are not limited to, ampoules or multi-dose containers.

According to some embodiments, the microparticulate formulation comprises a suspension of microparticles. According to some embodiments, the pharmaceutical composition further comprises at least one of a suspending agent, a stabilizing agent and a dispersing agent. According to some such embodiments, the pharmaceutical composition is presented as a suspension. According to some such embodiments, the pharmaceutical composition is presented as a solution. According to some such embodiments, the pharmaceutical composition is presented as an emulsion.

According to some embodiments, a formulation of the pharmaceutical composition comprises an aqueous solution of the therapeutic agent in water-soluble form. According to some embodiments, the formulation of the pharmaceutical composition comprises an oily suspension of the therapeutic agent. An oily suspension of the therapeutic agent can be prepared using suitable lipophilic solvents. Exemplary lipophilic solvents or vehicles include, but are not limited to, fatty oils such as sesame oil, or synthetic fatty acid esters, such as ethyl oleate or triglycerides. According to some embodiments, the formulation of the pharmaceutical composition comprises an aqueous suspension of the therapeutic agent. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the therapeutic agent(s) to allow for the preparation of highly concentrated solutions. Alternatively, the therapeutic agent can be in powder form for constitution with a suitable vehicle, e.g., sterile pyrogen-free water, before use.

Suitable liquid or solid pharmaceutical preparations include, for example, microencapsulated dosage forms, and if appropriate, with one or more excipients, encochleated, coated onto microscopic gold particles, contained in liposomes, pellets for implantation into the tissue, or dried onto an object to be rubbed into the tissue. As used herein, the term “microencapsulation” refers to a process in which very tiny droplets or particles are surrounded or coated with a continuous film of biocompatible, biodegradable, polymeric or non-polymeric material to produce solid structures including, but not limited to, nonpareils, pellets, crystals, agglomerates, microspheres, or nanoparticles. Such pharmaceutical compositions also can be in the form of granules, beads, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, or solubilizers are customarily used as described above. The pharmaceutical compositions are suitable for use in a variety of drug delivery systems.

### Microencapsulation Process

Examples of microencapsulation processes and products; methods for the production of emulsion-based microparticles; emulsion-based microparticles and methods for the production thereof; solvent extraction microencapsulation with tunable extraction rates; microencapsulation process with solvent and salt; a continuous double emulsion process for making microparticles; drying methods for tuning microparticle properties, controlled release systems from polymer blends; polymer mixtures comprising polymers having different non-repeating units and methods for making and using the same; and an emulsion based process for preparing microparticles and workhead assembly for use with same are disclosed and described in U.S. Pat. No. 5, 407,609 (entitled Microencapsulation Process and Products Thereof), U.S. application Ser. No. 10/553,003 (entitled Method for the production of emulsion-based microparticles), U.S. application Ser. No. 11/799,700 (entitled Emulsion-based microparticles and methods for the production thereof), U.S. application Ser. No. 12/557,946 (entitled Solvent Extraction Microencapsulation With Tunable Extraction Rates) , U.S. application Ser. No. 12/779,138 (entitled Hyaluronic Acid (HA) Injection Vehicle), U.S. application Ser. No. 12/562,455 entitled Microencapsulation Process With Solvent And Salt) , U.S. application Ser. No. 12/338,488 (entitled Process For Preparing Microparticles Having A Low Residual Solvent Volume); U.S. Application No. 12/692,027 (entitled Controlled Release Systems From Polymer Blends); U.S. Application No. 12/692,020 (entitled Polymer Mixtures Comprising Polymers Having Different Non-Repeating Units And Methods For Making And Using Same); U.S. application Ser. No. 10/565,401 (entitled “Controlled release compositions”); U.S. application Ser. No. 12/692,029 (entitled “Drying Methods for Tuning Microparticle Properties); U.S. application Ser. No. 12/968,708 (entitled “Emulsion Based Process for Preparing Microparticles and Workhead for Use with Same); and U.S. application Ser. No. 13/074,542 (entitled Composition and Methods for Improved Retention of a Pharmaceutical Composition at a Local Administration Site”) The contents of each of these are incorporated herein by reference in its entirety.

According to some embodiments, delivery of the therapeutic agent using microparticle technology involves bioresorbable, polymeric particles that encapsulate the therapeutic agent and optionally an additional therapeutic agent.

According to one embodiment, the microparticle formulation comprises a polymer matrix, wherein the therapeutic agent is impregnated in the polymer matrix. According to one embodiment, the polymer is a slow release polymer. According to one embodiment, the polymer is poly (D, L-Lactide-co-glycolide). According to another embodiment, the polymer is poly(orthoester). According to another embodiment, the polymer is poly(anhydride). According to another embodiment, the polymer is polylactide-polyglycolide.

Both non-biodegradable and biodegradable polymeric materials can be used in the manufacture of particles for delivering the therapeutic agent(s). Such polymers can be natural or synthetic polymers. The polymer is selected based on the period of time over which release is desired. Bioadhesive polymers of particular interest include, but are not limited to, bioerodible hydrogels as described by Sawhney et al in Macromolecules (1993) 26, 581-587, the teachings of which are incorporated herein. Exemplary bioerodible hydrogels include, but are not limited to, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). According to one embodiment, the bioadhesive polymer is hyaluronic acid. According to some such embodiments, the bioadhesive polymer include less than about 2.3% of hyaluronic acid.

According to some embodiments, the pharmaceutical composition is formulated for parenteral injection, implantation, topical administration, or a combination thereof. According to some such embodiments, the pharmaceutical composition is in the form of a pharmaceutically acceptable sterile aqueous or nonaqueous solution, dispersion, suspension or emulsion or a sterile powder for reconstitution into a sterile injectable solution or dispersion. Examples of suitable aqueous and nonaqueous carriers, diluents, solvents or vehicles include, but are not limited to, water, ethanol, dichloromethane, acetonitrile, ethyl acetate, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Suspensions can further contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

According to some embodiments, the pharmaceutical composition is formulated in an injectable depot form. Injectable depot forms are made by forming microencapsulated matrices of therapeutic agent in a biodegradable polymer. Depending upon the ratio of drug to polymer and the nature of the particular polymer employed, the rate of drug release may be controlled. Such long acting formulations can be formulated with suitable polymeric or hydrophobic materials (for example as an emulsion in an acceptable oil) or ion exchange resins, or as sparingly soluble derivatives, for example, as a sparingly soluble salt. Examples of biodegradable polymers include, but are not limited to, polylactide-polyglycolide, poly(orthoesters) and poly(anhydrides). Depot injectable formulations are also prepared by entrapping the drug in liposomes or microemulsions which are compatible with body tissues.

According to some embodiments, the therapeutic agent is impregnated in or on a polyglycolide (PGA) matrix. PGA is a linear aliphatic polyester developed for use in sutures. Studies have reported PGA copolymers formed with trimethylene carbonate, polylactic acid (PLA), and other polyesters, like polycaprolactone. Some of these copolymers may be formulated as microparticles for sustained drug release.

According to some embodiments, the therapeutic agent is impregnated in or on a polyester—polyethylene glycol matrix. Polyester—polyethylene glycol compounds can be synthesized; these are soft and may be used for drug delivery.

According to some embodiments, the therapeutic agent is impregnated in or on a poly (amino)-derived biopolymer matrix. Poly (amino)-derived biopolymers can include, but are not limited to, those containing lactic acid and lysine as the aliphatic diamine (see, for example, U.S. Pat. No. 5,399,665), and tyrosine-derived polycarbonates and polyacrylates. Modifications of polycarbonates may alter the length of the alkyl chain of the ester (ethyl to octyl), while modifications of polyarylates may further include altering the length of the alkyl chain of the diacid (for example, succinic to sebasic), which allows for a large permutation of polymers and great flexibility in polymer properties.

According to some embodiments, the therapeutic agent is impregnated in or on a polyanhydride matrix. Polyanhydrides are prepared by the dehydration of two diacid molecules by melt polymerization (see, for example, U.S. Pat. No. 4,757,128). These polymers degrade by surface erosion (as compared to polyesters that degrade by bulk erosion). The release of the drug can be controlled by the hydrophilicity of the monomers chosen.

According to some embodiments, the therapeutic agent is impregnated in or on a photopolymerizable biopolymer matrix. Photopolymerizable biopolymers include, but are not limited to, lactic acid/polyethylene glycol/acrylate copolymers.

According to some embodiments, the therapeutic agent is impregnated in or on a hydrogel matrix. The term “hydrogel” refers to a substance resulting in a solid, semisolid, pseudoplastic or plastic structure containing a necessary aqueous component to produce a gelatinous or jelly-like mass. Hydrogels generally comprise a variety of polymers, including hydrophilic polymers, acrylic acid, acrylamide and 2-hydroxyethylmethacrylate (HEMA).

According to some embodiments, the therapeutic agent is impregnated in or on a naturally-occurring biopolymer matrix. Naturally-occurring biopolymers include, but are not limited to, protein polymers, collagen, polysaccharides, and photopolymerizable compounds.

According to some embodiments, the therapeutic agent is impregnated in or on a protein polymer matrix. Protein polymers have been synthesized from self-assembling protein polymers such as, for example, silk fibroin, elastin, collagen, and combinations thereof.

According to some embodiments, the therapeutic agent is impregnated in or on a naturally-occurring polysaccharide matrix. Naturally-occurring polysaccharides include, but are not limited to, chitin and its derivatives, hyaluronic acid, dextran and cellulosics (which generally are not biodegradable without modification), and sucrose acetate isobutyrate (SAIB).

According to some embodiments, the therapeutic agent is impregnated in or on a chitin matrix. Chitin is composed predominantly of 2-acetamido-2-deoxy-D-glucose groups and is found in yeasts, fungi and marine invertebrates (shrimp, crustaceous) where it is a principal component of the exoskeleton. Chitin is not water soluble and the deacetylated chitin, chitosan, only is soluble in acidic solutions (such as, for example, acetic acid). Studies have reported chitin derivatives that are water soluble, very high molecular weight (greater than 2 million daltons), viscoelastic, non-toxic, biocompatible and capable of crosslinking with peroxides, gluteraldehyde, glyoxal and other aldehydes and carbodiamides, to form gels.

According to some embodiments, the therapeutic agent is impregnated in or on a hyaluronic acid (HA) matrix. Hyaluronic acid (HA), which is composed of alternating glucuronidic and glucosaminidic bonds and is found in mammalian vitreous humor, extracellular matrix of the brain, synovial fluid, umbilical cords and rooster combs from which it is isolated and purified, also can be produced by fermentation processes.

According to some embodiments, the pharmaceutical composition further comprises an adjuvant. Exemplary adjuvants include, but are not limited to, preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. Isotonic agents, for example, sugars, sodium chloride and the like, can also be included. Prolonged absorption of the injectable pharmaceutical form can be brought about by the use of agents delaying absorption, for example, aluminum monostearate and gelatin.

The formulations can be sterilized, for example, by terminal gamma irradiation, filtration through a bacterial-retaining filter or by incorporating sterilizing agents in the form of sterile solid compositions that may be dissolved or dispersed in sterile water or other sterile injectable medium just prior to use. Injectable preparations, for example, sterile injectable aqueous or oleaginous suspensions may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol, dichloromethane, ethyl acetate, acetonitrile, etc. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose any bland fixed oil may be employed including synthetic mono- or diglycerides. In addition, fatty acids such as oleic acid are used in the preparation of injectables.

Formulations for parenteral (including but not limited to, intraocular, intraorbital, subconjunctival, subcutaneous, intradermal, intramuscular, intravenous, intraarterial, intrathecal, intraventricular and intraarticular) administration include aqueous and non-aqueous sterile injection solutions that can contain anti-oxidants, buffers, bacteriostats and solutes, which render the formulation isotonic with the blood of the intended recipient; and aqueous and non-aqueous sterile suspensions, which can include suspending agents and thickening agents.

According to another embodiment, the pharmaceutical composition is formulated by conjugating the therapeutic agent to a polymer that enhances aqueous solubility. Examples of suitable polymers include but are not limited to polyethylene glycol, poly-(d-glutamic acid), poly-(1-glutamic acid), poly-(1-glutamic acid), poly-(d-aspartic acid), poly-(1-aspartic acid), poly-(1-aspartic acid) and copolymers thereof. Polyglutamic acids having molecular weights between about 5,000 to about 100,000, with molecular weights between about 20,000 and about 80,000 may be used and with molecular weights between about 30,000 and about 60,000 may also be used. The polymer is conjugated via an ester linkage to one or more hydroxyls using a protocol as essentially described by U.S. Pat. No. 5,977,163 which is incorporated herein by reference.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

As used herein, the singular forms “a”, “an” and “the” include plural referents unless the context clearly dictates otherwise. For example, reference to a “polypeptide” means one or more polypeptides.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and described the methods and/or materials in connection with which the publications are cited.

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Effect of a Nimodipine formulation on Glaucoma Patients

A test formulation of a particulate nimodipine formulation containing a uniform distribution of microparticle size will be prepared by combining a polymer solution (e.g., a 50-50 glycolide-lactide blend) with a solvent in the presence of nimodipine. The mixture will be added to a surfactant containing aqueous solution to form an emulsion and the solvent extracted to produce the flowable microparticulate nimodipine formulation. The initial drug load will be 65%, i.e., 65% nimodipine and 35% polymer. The mean particle size will be about 52 μm.

The microparticulate nimodipine formulation can be combined with an additional therapeutic agent, e.g., a prostaglandin analog, Rho kinase inhibitor and a pharmaceutical carrier to form the pharmaceutical composition of the described invention. A vehicle (e.g. saline (hydroxyl propyl methyl cellulose (HPMC) in phosphate buffered saline (PBS))) can be mixed with the microparticulate nimodipine formulation. A placebo formulation containing the microparticles created with no nimodipine plus vehicle will be prepared.

### Treatment Groups

### Inclusion Criteria

Glaucoma patients will be included in this study. Inclusion criteria will be glaucoma defined as pathological optic disc appearance and pathological visual field as evidenced from automated perimetry (e.g., Humphrey Field analyser program 30-2) as assessed on the study day. Pathological visual field testing will be defined according to the criteria of the Ocular Hypertension Treatment Study: glaucoma hemifield test outside normal and/or a CPSD with p<0.05 (Keltner J L et al., Arch Ophthalmol 2003; 121:643-50). All patients will have performed at least three visual field tests in total and one test within the last 6 months before the start of the study. Further inclusion criteria will be visual acuity better than 20/30 and ametropia <3 diopters. None of the included patients will have a history of IOP >21 mm Hg without antiglaucoma therapy and will be verified by at least one diurnal tension curve, which will have been recorded no more than one year before inclusion in the present study. A washout period of three weeks will be scheduled for patients with antiglaucomatous therapy or with intake of magnesium or ginkgo biloba preparations.

### Exclusion Criteria

Patients will be excluded if they have a history of glaucoma surgery or any sign of another relevant retinal eye disease. Patients with uncontrolled systemic hypertension of more than 150/90 mm Hg or medication with systemic calcium channel antagonists will also be excluded from the trial. Patients with a history of TOP >22 mm Hg by Goldmann applanation tonometer or similar method, those with chronic or recurrent history of ocular inflammation, other ocular diseases that would impair assessment of visual fields, and contraindications to nimodipine will be excluded. Patients with a history of IOP >22 mm Hg by Goldmann applanation tonometer or similar method, those with chronic or recurrent history of ocular inflammation, other ocular diseases that would impair assessment of visual fields, and contraindications to nimodipine will be excluded. All patients will undergo a standardized cold-warm challenge test using infrared telethermography to quantify Raynaud's phenomenon.

### Administration

The control (particulate Placebo Formulation) and test articles (particulate Nimodipine Formulation) can be administered once on Day 1 topically, by injection or by implantation. The dose levels for the treated groups will be 10 mg or 30 mg at a fixed dose volume of 0.25 mL (Microparticulate Placebo Formulation), 0.17 mL or 0.18 mL (Microparticulate Nimodipine Formulation at Low Dose), or 0.46 mL (Microparticulate Nimodipine Formulation at High Dose).

### Study Design

The study will follow a randomized, placebo controlled, double-blind design. On the first study day, patients will be randomized to receive either Microparticulate Nimodipine Formulation at Low Dose or at High Dose or Microparticulate Placebo Formulation. On the study day, a resting period of at least 20 minutes will be scheduled to ensure stabile haemodynamic conditions which will be verified by repeated blood pressure measurements. Baseline measurements of fundus pulsations, laser Doppler flowmetry, color contrast sensitivity, intraocular pressure and systemic haemodynamics will be performed. After completion of these measurements, patients will receive either Nimodipine or Placebo Formulations. All measurements will be performed again based on the pharmacokinetics of the Nimodipine Formulation.

### Materials and Methods

### Systemic Hemodynamics

Systolic, diastolic, and mean blood pressures (SBP, DBP, MAP) will be measured on the upper arm by an automated oscillometric device. Pulse rate (PR) will be automatically recorded from a finger pulse oxymetric device (HP-CMS patient monitor, Hewlett Packard, Palo Alto, Calif., USA).

### Laser Doppler Flowmetry (LDF)

Choroidal and ONHBF will be assessed with LDF according to Riva et al (Oculix 4000, Oculix Sarl, Arbaz, Switzerland) (Exp Eye Res 1992; 55:499-506; Invest Ophthalmol Vis Sci 1994; 35:4273-81). The principles of LDF have been described in detail by Bonner and Nossal (Shepard A P, Oberg A P, Vol. 107 of Developments in Cardiovascular Medicine, Boston; Kluwer Academic Publishers, 1990:17-45). Briefly, the vascularised tissue is illuminated by coherent laser light. Scattering on moving red blood cells (RBCs) leads to a frequency shift in the scattered light. In contrast, static scatterers in tissue do not change light frequency but lead to randomization of light directions impinging on RBCs. This light diffusing in vascularised tissue leads to a broadening of the spectrum of scattered light (Doppler shift power spectrum, DSPS). From this DSPS the mean RBC velocity, the blood volume, and the blood flow can be calculated in relative units. For example, the laser beam can be directed to the fovea to assess blood flow in the submacular choroid (CHBF). Blood flow in the ONH can be measured at the temporal neuroretinal rim (ONHBF).

### Fundus Pulsation Technique

Ocular fundus pulsation can be assessed by laser interferometry as described by Schmetterer et al (Opt Eng 1995; 34:711-6). Briefly, the eye is illuminated by the beam of a single mode laser diode (=783 nm) along the optical axis. The light is reflected at both the front surface of the cornea and the retina. The two re-emitted waves produce interference fringes from which the distance changes between cornea and retina during a cardiac cycle can be calculated. These distance changes are caused by the pulsatile inflow of blood through the arteries and by the non-pulsatile outflow through the veins. The maximum change in corneo-retinal distance is called fundus pulsation amplitude (FPA). This method has been shown to estimate the pulsatile blood flow in the choroidal vasculature (Schmetterer L et al., Invest Ophtalmol Vis Sci 1998; 39:1210-20). Measurements will be performed in the fovea.

### Measurement of Intraocular Pressure

A Goldmann applanation tonometer will be used to measure intraocular pressure (TOP).

### Peripheral Color Contrast Sensitivity (Threshold Along Tritan Axis)

Peripheral color contrast sensitivity will be measured with a computer graphics device (Yu T C et al., Invest Opthalmol Vis Sci 1991; 32:2779-89). A program calculates the relative voltages required to produce any color in terms of color space. A high definition color monitor driven by a personal computer with a graphics interface card displays an annulus subtending 25° at the eye. The program produces images without spatial luminance variations to test color contrast. The color contrast between the annulus and the background can be varied. Forty five degrees of the annulus is randomly removed in one of four quadrants. Patients are asked to identify the position of the gap while fixating a central spot. The minimum color contrast between annulus and background at which the identification is possible is between 13-16% for the protan, deuteran, and tritan axis in normal subjects. This threshold value changes little with age, refractive error, or pupillary aperture, and test-retest variability is low. Modulation is done along color confusion lines for trichromatic vision (protan, deutan, tritan). Contrast sensitivity will be determined in 20° off axis along the tritan colour axis based on the results of previous studies.

### Infrared Telethermography and Assessment of Raynaud's Phenomenon

Raynaud's Phenomenon is excessively reduced blood flow in response to cold or emotional stress. In order to assess this condition in patients, continuous temperature recordings of all 10 fingers will be done during standardized provocation tests using a previously described program (Black C M et al., J Physiol 1987; 384:6p). Mean room air temperature will be kept at 22.0° C. (SD 0.5° C.). After adaptation to room air for at least 20 minutes basal fingertip skin temperature will be measured. Thereafter a 1 minute warm challenge will be induced by immersion of gloved hands in water at 39° C. and recovery temperature will be assessed 10 and 20 minutes thereafter. A second stimulation will consist of a 1 minute cold challenge by inducing immersion of gloved hands in water at 20° C. Temperatures will be measured 10 and 20 minutes after this cold provocation test. Raynaud's phenomenon will be diagnosed as having a positive test and a clear history of cold hands.

### Data Analysis

For data analysis the absolute values will be chosen. The effects of nimodipine on hemodynamic variables and TOP will be assessed with repeated measure analysis of variance (ANOVA) versus placebo. The percentage change over baseline in response to nimodipine and placebo will be calculated. The association between percentage changes in ocular hemodynamic parameters and percentage changes in threshold will be analysed with linear regression analysis. Data will be presented as mean (standard deviation). A p value of less than 0.05 will be considered the level of significance.

### Equivalents

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

What is claimed is:

1. A method for treating at least one adverse consequence of an eye disease comprising abnormal intraocular pressure, retinal vascular disease and retinal ganglion cell death in order to reduce visual loss in a mammal in need thereof, the method comprising:
(a) providing a flowable particulate composition comprising
(i) a particulate formulation comprising a plurality of particles of uniform size distribution, and a therapeutic amount of a therapeutic agent selected from a voltage-gated calcium channel antagonist, an endothelin receptor antagonist, or a combination thereof, and optionally an additional therapeutic agent, wherein the particles are of uniform size distribution, and wherein each particle comprises a matrix; and
(ii) a pharmaceutically acceptable carrier,
the pharmaceutical composition being characterized by:
dispersal of the therapeutic agent throughout each particle, adsorption of the therapeutic agent onto the particles, or placement of the therapeutic agent in a core surrounded by a coating,
sustained release of the therapeutic agent and optionally the additional agent from the composition, and
a local therapeutic effect that is effective to reduce signs or symptoms of the adverse consequence without entering systemic circulation in an amount to cause unwanted side effects; and
(b) administering a therapeutic amount of the pharmaceutical composition by a means for administration at a site of administration.

2. The method according to claim 1, wherein the adverse consequence of the eye disease comprises abnormal intraocular pressure.

3. The method according to claim 1, wherein the adverse consequence of the eye disease comprises retinal ganglion cell death.

4. The method according to claim 1, wherein the adverse consequence of the eye disease comprises a retinal vascular disease.

5. The method according to claim 4, wherein the retinal vascular disease is glaucoma.

6. The method according to claim 1, wherein the voltage-gated calcium channel antagonist is a dihydropyridine.

7. The method according to claim 6, wherein the dihydropyridine is nimodipine.

8. The method according to claim 1, wherein the additional therapeutic agent is a prostaglandin analog, a Rho kinase inhibitor, or a combination thereof.

9. The method according to claim 8, wherein the prostaglandin analog is latanoprost, bimatoprost, or travaprost.

10. The method according to claim 8, wherein the Rho kinase inhibitor is selected from the group consisting of Y-27632 2HCl (R&D Systems Inc., Minneapolis, Minn.), Triazovivin® (StemRD, Burlingame, Calif.), Slx-2119 (MedChem Express, Namiki Shoji Cop., LTD), WF-536 [(+)-©-4-(1-aminoethyl)-N-(4-pyridyl) benzamide monohydrochloride] (Mitsubishi Pharma Corporation, Osaka, Japan), RK1-1447 (University of South Florida, Tampa, Fla., and Moffitt Cancer Center, Tampa, Fla.; Roberta Pireddu et al., “Pyridylthiazole-based ureas as inhibitors of Rho associated protein kinases (ROCK1 and 2).” (2012) Medchemcomm. 3(6):699-709), Fasudil® (Asahi-KASEI Corp., Osaka, Japan), Fasudil® hydrochloride (R&D Systems Inc., Minneapolis, Minn.), GSK429286A (R&D Systems Inc., Minneapolis, Minn.), Rockout® (EMD Millipore, Philadelphia, Pa.), SR 3677 dihydrochloride (R&D Systems Inc., Minneapolis, Minn.); SB 772077B (R&D Systems Inc., Minneapolis, Minn.), AS 1892802 (R&D Systems Inc., Minneapolis, Minn.), H 1152 dihydrochloride (R&D Systems Inc., Minneapolis, Minn.), GSK 269962 (R&D Systems Inc., Minneapolis, Minn.), HA 1100 hydrochloride (R&D Systems Inc., Minneapolis, Minn.), Glycyl-H-1152 dihydrochloride (R&D Systems Inc., Minneapolis, Minn.), AR-12286 (Aerie Pharmaceuticals), AR-13324 (Rhopressa, Aerie Pharmaceuticals), AMA-0076 (Amakem Therapeutics), and K-115 (Kumatomo University, Japan).

11. The method according to claim 1, wherein the administering is topically, parenterally, or by implantation.

12. The method according to claim 11, wherein the administering is intraocularly, intraorbitally or into the subconjunctival space.

13. The method according to claim 12, wherein administering intraocularly comprises administering to the vitreous humor, the aqueous humor, or both.

14. A kit for treating at least one adverse consequence of an eye disease comprising abnormal intraocular pressure, retinal vascular disease and retinal ganglion cell death in order to reduce visual loss comprising:
(a) a flowable particulate composition comprising
(i) a particulate formulation comprising a plurality of particles of uniform size distribution, a therapeutic amount of a therapeutic agent selected from a voltage-gated calcium channel antagonist, an endothelin receptor antagonist, or a combination thereof, and optionally an additional therapeutic agent, wherein the microparticles are of uniform size distribution, and wherein each microparticle comprises a matrix,
the pharmaceutical composition being characterized by:
dispersal of the therapeutic agent throughout each particle, adsorption of the therapeutic agent onto the particles, or placement of the therapeutic agent in a core surrounded by a coating,
sustained release of the voltage-gated calcium channel antagonist, the endothelin receptor antagonist, or the combination thereof and optionally an additional therapeutic agent, from the composition, and
a local therapeutic effect that is effective to reduce signs or symptoms of the adverse consequence selected from abnormal intraocular pressure, retinal vascular disease and retinal ganglion cell death without entering systemic circulation in an amount to cause unwanted side effects;
(b) a means for administering the pharmaceutical composition; and
(c) a packaging material.

15. The kit according to claim 14, wherein the voltage-gated calcium channel antagonist is dihydropyridine.

16. The kit according to claim 15, wherein the dihydropyridine is nimodipine.

17. The kit according to claim 14, wherein the pharmaceutical composition further comprises a pharmaceutically acceptable carrier.

18. The kit according to claim 14, wherein the packaging material is an instruction.

19. The kit according to claim 14, wherein the means for administering the pharmaceutical composition comprises a syringe, an eye dropper, or a contact lens.

20. The kit according to claim 19, wherein the contact lens is selected from the group consisting of a soft contact lens, a gas permeable contact lens, and a hybrid contact lens.

21. The kit according to claim 14, wherein the composition is in a form of a sheet, a string, or a combination thereof.

22. The kit according to claim 21, wherein the sheet, the string, or a combination thereof is impregnated with the composition.
